SEQUENCE LISTING (56 sequences)

SEQ ID NO: 1 - DNA, 798 bp, synthetic construct
  source 1..798: mol_type = other DNA
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttgg

SEQ ID NO: 2 - DNA, 222 bp, synthetic construct
  source 1..222: mol_type = other DNA
gtctgtctgcacatttcgtagagcgagtgttccgatactctaatctccctaggcaaggttcatatttgtgtaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcct

SEQ ID NO: 3 - DNA, 222 bp, synthetic construct
  source 1..222: mol_type = other DNA
gtctgtctgcacatttcgtagagcgagtgttccgatactctaatctccctaggcaaggttcatattgacttaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcct

SEQ ID NO: 4 - DNA, 239 bp, synthetic construct
  source 1..239: mol_type = other DNA
ccaccccctccaccttggacacaggacgctgtggtttctgagccaggtacaatggggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaat

SEQ ID NO: 5 - DNA, 396 bp, synthetic construct
  source 1..396: mol_type = other DNA
gatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaa

SEQ ID NO: 6 - DNA, 439 bp, synthetic construct
  source 1..439: mol_type = other DNA
tgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagagat

SEQ ID NO: 7 - DNA, 72 bp, synthetic construct
  source 1..72: mol_type = other DNA
gggggaggctgctggtgaatattaaccaaggtcaccccagttatcggaggagcaaacaggggctaagtccac

SEQ ID NO: 8 - DNA, 216 bp, synthetic construct
  source 1..216: mol_type = other DNA
gggggaggctgctggtgaatattaaccaaggtcaccccagttatcggaggagcaaacaggggctaagtccacgggggaggctgctggtgaatattaaccaaggtcaccccagttatcggaggagcaaacaggggctaagtccacgggggaggctgctggtgaatattaaccaaggtcaccccagttatcggaggagcaaacaggggctaagtccac

SEQ ID NO: 9 - DNA, 432 bp, synthetic construct
  source 1..432: mol_type = other DNA
gggggaggctgctggtgaatattaaccaaggtcaccccagttatcggaggagcaaacaggggctaagtccacgggggaggctgctggtgaatattaaccaaggtcaccccagttatcggaggagcaaacaggggctaagtccacgggggaggctgctggtgaatattaaccaaggtcaccccagttatcggaggagcaaacaggggctaagtccacgggggaggctgctggtgaatattaaccaaggtcaccccagttatcggaggagcaaacaggggctaagtccacgggggaggctgctggtgaatattaaccaaggtcaccccagttatcggaggagcaaacaggggctaagtccacgggggaggctgctggtgaatattaaccaaggtcaccccagttatcggaggagcaaacaggggctaagtccac

SEQ ID NO: 10 - DNA, 118 bp, synthetic construct
  source 1..118: mol_type = other DNA
gttaatttttaaaaagcaagtggcccttggcagcatctgtttgctctggttaataatctcaggagcacaaacattcccaggagaagaaatcaacatcctggacttatcctctgggcct

SEQ ID NO: 11 - DNA, 116 bp, synthetic construct
  source 1..116: mol_type = other DNA
gcactgggaggatgttgagtaagatggaaaactactgatgacccttgcagagacagagtattaggacatgtttgaacaggggccgggcgatcagcaggtagctctagaggatcccc

SEQ ID NO: 12 - DNA, 232 bp, synthetic construct
  source 1..232: mol_type = other DNA
gcactgggaggatgttgagtaagatggaaaactactgatgacccttgcagagacagagtattaggacatgtttgaacaggggccgggcgatcagcaggtagctctagaggatccccgcactgggaggatgttgagtaagatggaaaactactgatgacccttgcagagacagagtattaggacatgtttgaacaggggccgggcgatcagcaggtagctctagaggatcccc

SEQ ID NO: 13 - DNA, 321 bp, synthetic construct
  source 1..321: mol_type = other DNA
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggg

SEQ ID NO: 14 - DNA, 642 bp, synthetic construct
  source 1..642: mol_type = other DNA
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggg

SEQ ID NO: 15 - DNA, 774 bp, synthetic construct
  source 1..774: mol_type = other DNA
ctgcaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtccgggttcaaaaccacttgctgggtggggagtcgtcagtaagtggctatgccccgaccccgaagcctgtttccccatctgtacaatggaaatgataaagacgcccatctgatagggtttttgtggcaaataaacatttggtttttttgttttgttttgttttgttttttgagatggaggtttgctctgtcgcccaggctggagtgcagtgacacaatctcatctcaccacaaccttcccctgcctcagcctcccaagtagctgggattacaagcatgtgccaccacacctggctaattttctatttttagtagagacgggtttctccatgttggtcagcctcagcctcccaagtaactgggattacaggcctgtgccaccacacccggctaattttttctatttttgacagggacggggtttcaccatgttggtcaggctggtctaga

SEQ ID NO: 16 - DNA, 682 bp, synthetic construct
  source 1..682: mol_type = other DNA
caggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagat

SEQ ID NO: 17 - DNA, 726 bp, synthetic construct
  source 1..726: mol_type = other DNA
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaa

SEQ ID NO: 18 - DNA, 1494 bp, synthetic construct
  source 1..1494: mol_type = other DNA
gcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtga

SEQ ID NO: 19 - DNA, 1611 bp, synthetic construct
  source 1..1611: mol_type = other DNA
atggagttttcaagtccttccagagaggaatgtcccaagcctttgagtagggtaagcatcatggctggcagcctcacaggattgcttctacttcaggcagtgtcgtgggcatcaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggaatcggatataacatcatccgggtacccatggccagctgtgacttctccatccgcacctacacctatgcagacacccctgatgatttccagttgcacaacttcagcctcccagaggaagataccaagctcaagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacgaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcagcaagttcattcctgagggctcccagagagtggggctggttgccagtcagaagaacgacctggacgcagtggcactgatgcatcccgatggctctgctgttgtggtcgtgctaaaccgctcctctaaggatgtgcctcttaccatcaaggatcctgctgtgggcttcctggagacaatctcacctggctactccattcacacctacctgtggcgtcgccagtga

SEQ ID NO: 20 - DNA, 1494 bp, synthetic construct
  source 1..1494: mol_type = other DNA
gcccggccctgcatcccaaagagctttggctacagctctgtggtgtgcgtgtgcaacgccacctactgcgacagtttcgaccctcccaccttcccagccctgggcacattctctcggtacgagtccactcgctcaggccggagaatggagctgtctatgggcccaatccaggctaatcataccggaacaggcctgctgctgacactgcagccagagcagaagtttcagaaggtgaaaggcttcgggggcgccatgaccgatgctgctgccctgaatatcctggccctgagtcccccagctcagaatctgctgctgaagtcatactttagtgaggaaggcattggatacaatattatccgggtgcctatggcctcctgtgacttctcaattcgcacctatacttacgctgatacccccgatgatttccagctgcacaacttcagtctgcccgaggaagataccaaactgaaaatcccactgatccatcgggctctgcagctggctcagcgcccagtgagcctgctggcctctccttggacctcaccaacatggctgaagactaacggcgccgtgaacgggaaaggcagtctgaaagggcagccaggcgacatctatcaccagacttgggcccggtacttcgtgaaatttctggatgcctacgctgagcacaaactgcagttttgggccgtgaccgccgaaaatgagccatcagccggcctgctgtctggctacccatttcagtgtctggggtttacaccagagcatcagagggacttcatcgcccgggacctgggaccaaccctggccaacagcacccaccataacgtgaggctgctgatgctggacgatcagcggctgctgctgcctcactgggctaaggtggtgctgaccgatccagaggctgccaagtacgtgcatggaatcgccgtgcactggtacctggatttcctggcccctgctaacgccaccctgggagagacccatcggctgttccccaacaccatgctgtttgcctctgaagcttgcgtggggtcaaagttttgggagcagtcagtgaggctgggcagttgggatcgggggatgcagtactcccacagtattatcaccaatctgctgtaccatgtggtgggctggaccgattggaatctggccctgaatccagaggggggccctaattgggtgcggaacttcgtggactccccaatcattgtggatattacaaaggacaccttctacaaacagcccatgttctaccacctgggccactttagtaagttcatccctgaggggtcacagcgggtgggcctggtggcctcccagaaaaacgacctggatgctgtggctctgatgcaccctgacggatcagccgtggtggtggtgctgaatcgctctagtaaggacgtgccactgaccatcaaggatccagctgtgggcttcctggagacaatcagtcccgggtactctatccacacctatctgtggcgccggcagtga

SEQ ID NO: 21 - DNA, 1611 bp, synthetic construct
  source 1..1611: mol_type = other DNA
atggagttctcaagtccttctcgcgaggaatgtcccaaaccactgagccgcgtgtcaattatggctggatctctgaccggactgctgctgctgcaggccgtgagttgggctagtggggcccggccctgcatcccaaagagctttggctacagctctgtggtgtgcgtgtgcaacgccacctactgcgacagtttcgaccctcccaccttcccagccctgggcacattctctcggtacgagtccactcgctcaggccggagaatggagctgtctatgggcccaatccaggctaatcataccggaacaggcctgctgctgacactgcagccagagcagaagtttcagaaggtgaaaggcttcgggggcgccatgaccgatgctgctgccctgaatatcctggccctgagtcccccagctcagaatctgctgctgaagtcatactttagtgaggaaggcattggatacaatattatccgggtgcctatggcctcctgtgacttctcaattcgcacctatacttacgctgatacccccgatgatttccagctgcacaacttcagtctgcccgaggaagataccaaactgaaaatcccactgatccatcgggctctgcagctggctcagcgcccagtgagcctgctggcctctccttggacctcaccaacatggctgaagactaacggcgccgtgaacgggaaaggcagtctgaaagggcagccaggcgacatctatcaccagacttgggcccggtacttcgtgaaatttctggatgcctacgctgagcacaaactgcagttttgggccgtgaccgccgaaaatgagccatcagccggcctgctgtctggctacccatttcagtgtctggggtttacaccagagcatcagagggacttcatcgcccgggacctgggaccaaccctggccaacagcacccaccataacgtgaggctgctgatgctggacgatcagcggctgctgctgcctcactgggctaaggtggtgctgaccgatccagaggctgccaagtacgtgcatggaatcgccgtgcactggtacctggatttcctggcccctgctaacgccaccctgggagagacccatcggctgttccccaacaccatgctgtttgcctctgaagcttgcgtggggtcaaagttttgggagcagtcagtgaggctgggcagttgggatcgggggatgcagtactcccacagtattatcaccaatctgctgtaccatgtggtgggctggaccgattggaatctggccctgaatccagaggggggccctaattgggtgcggaacttcgtggactccccaatcattgtggatattacaaaggacaccttctacaaacagcccatgttctaccacctgggccactttagtaagttcatccctgaggggtcacagcgggtgggcctggtggcctcccagaaaaacgacctggatgctgtggctctgatgcaccctgacggatcagccgtggtggtggtgctgaatcgctctagtaaggacgtgccactgaccatcaaggatccagctgtgggcttcctggagacaatcagtcccgggtactctatccacacctatctgtggcgccggcagtga

SEQ ID NO: 22 - protein, 497 aa, synthetic construct
ARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPANATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 23 - protein, 536 aa, synthetic construct
MEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPANATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 24 - DNA, 368 bp, synthetic construct
  source 1..368: mol_type = other DNA
gtaagagagagagctcccaatcagcattgtcacagtgcttctggaatcctggcactggaatttaatgaatgacagactctctttgaatccagggccatcatggctctttgagcaaggcacagatggagggaggggtcgaagttgaaatgggtgggaagagtggtggggagcatcctgatttggggtgggcagagagttgtcatcagaagggttgcagggagagctgcacccaggtttctgtgggccttgtcctaatgaatgtgggagaccgggccatgggcacccaaaggcagctaagccctgcccaggagagtagttgaggggtggagaggggcttgcttttcagtcattcctcattctgtcctcag

SEQ ID NO: 25 - DNA, 552 bp, synthetic construct
  source 1..552: mol_type = other DNA
gtgagtgagtcaaggcagtggggaggtagcacagagcctcccttctgcctcatagtcctttggtagccttccagtaagctggtggtagacttttagtaggtgctcaataaatccttttgagtgactgagaccaactttggggtgaggattttgttttttttcttttgaaacagagtcttactctgttgcctgggctggagtgcagtggtgcaattttggctcattccaacctctgcctcccagattcaagcgattctcttgcttcagcttcccaggtagctgggattacaggcggccaccactacgcccagctaatttttgtatttttagtagagacggggtttcaccatgctggcaaggcaggtctcaaactcctcacctcaggtgatccgcccacctcggcctcctaaagtgctaggattacaggtgtgagcccctgcgcccggccaaggggtgaggaattttgaaaccgtgttcagtctctcctagcagatgtgtccattctccatgtcttcatcagacctcactctgcttgtactccctccctcccag

SEQ ID NO: 26 - DNA, 123 bp, synthetic construct
  source 1..123: mol_type = other DNA
gtaaccattacacccctcaccccctgggccaggctgggtcctcctagaggtaaatggtgtcagtgatcaccatggagtttcccgctgggtactgatacccttattccctgtggatgtcctcag

SEQ ID NO: 27 - DNA, 965 bp, synthetic construct
  source 1..965: mol_type = other DNA
gtgaggaggaaggggacaagatgacatagagccattgaaacttttcgtttttcttttctttttttaaaatttttttgaggcagaatctcactctgcccattctgtcggcgagacaggagtgcagtggtgtgatctcccctcacagcaacctctgcctcccaggctatagtgattctcctgcctcagcctcctgagtagctggaattataggcgtgcgccactaccacctggctaatttttgtatttttagtagagacagggtttcatcatgttgaccaggctagtcttaaactcctgacctcaaatgatatacctgccttggcctcccgaagtgctggaattacaagtgtgagccaccgagcccagcagacacttttcttttttctttttttttttttgagacagagtctcgcactgtcacccaggctggagtgcagtggcacaatctcagctcactgcaacctccacctcccgggttcaggtgattctcctgtctcagcctctcgagtacctgggattacaggtgcctgccaccacgcccggctaattttttgtatttttagtagagacagggtttcactatgttggccaggatgattgcgaactcctgacctcgtgatctgcccacatcggcctcccaaagtgctgggattacatgcgtgagccactgacacttttctttgccctttctttggaccctgacttctgcccatccctgacatttggttcctgttttaatgccctgtgaaataagatttcaccgcctatcatctgctaactgctacggactcaggctcagaaaggcctgcgcttcacccaggtgccagcctccacaggttccaacccaggagcccaagttccctttggccctgactcagacactattaggactggcaagtgataagcagagtcccatactctcctattgactcggactaccatatcttgatcatccttttctgtag

SEQ ID NO: 28 - DNA, 210 bp, synthetic construct
  source 1..210: mol_type = other DNA
gtaggcattctagctttttcaggccctgagggccctgatgtctgggggttgagaaactgtagggtaggtctgcttgtacagacattttgtcccctgctgttttgtcctgggggtgggagggtggaggctaatggctgaaccggatgcactggttgggctagtatgtgttccaactctgggtgcttctctcttcactacctttgtctctag

SEQ ID NO: 29 - DNA, 555 bp, synthetic construct
  source 1..555: mol_type = other DNA
gtaagggatcagcaaggatgtgggatcaggactggcctcccatttagccatgctgatctgtgtcccaaccctcaacctagttccacttccagatctgcctgtcctcagctcacctttctaccttctgggcctttcagccttgggcctgtcaatcttgcccactccatcaggcttcctgttctctcggtctggcccactttctttttatttttcttcttttttttttttttgagaaggagtctctctctctgtcacccaggctggagtgctgtggcgccatcttcactcactgtaacctctgcctcctgagttcaagcaattctcctgcctcagccttccaagtagctgggattataggcgcctgccaccaggcccagctgatttttctatttttagtagagacggggtttcgccaggctgttctcgaactcctgaactcaagtgatccacctgcctcggcttcccaaagtgctgggattacaggtgtgagccaccacacccagctggtctggtccactttcttggccggatcattcatgacctttctcttgccag

SEQ ID NO: 30 - DNA, 871 bp, synthetic construct
  source 1..871: mol_type = other DNA
gtaaggcctggacctccatggtgctccagtgaccttcaaatccagcatccaaatgactggctcccaaacttagagcgatttctctacccaactatggattcctagagcaccattcccctggacctccagggtgccatggatcccacagttgtcgcttgaaacctttctaggggctgggcgaggtggctcactcatgcaaacccagcactttgggaagccgaggcgggtgatcacctgaggtcaggagtttaagaccaccctggccaacgtgttgaaaccctgtgtctactaaaatacaaaaaaaaaaaattatctgggcatgatggtgggtgtctgtaatcccagctactcaggaggctgagaagggagaatcagttgaacccgggagatggtggttgcggtgagccgagatcgcgccactgcactccagcctgggaggctgagcgagactccatctcgaaacaaaacaaaacaaaactatctaggctgggggtggtggttcatgtatgtatgtgtatatacatatatatgtgtttatatgtatatatatatacacacacacacatacatacacacacatacacacacaaattagctgggtgtggcacccgtgtagtcccagctactcaggaggctaatgtgggaggatcagttgaccctaggaagtcaaggctgcagtgagtcgtgattgcgccactgtactccagcccgagtgacagagtgacatcctgtctcaaaaacaaaaaaaaatctccccaaacctctctagttgcattcttcccgtcacccaactccaggattcctacaacaggaactagaagttccagaagcctgtgtgcaaggtccaggatcagttgctcttcctttgcag

SEQ ID NO: 31 - DNA, 400 bp, synthetic construct
  source 1..400: mol_type = other DNA
gtaagccaccccagtctcccttcctgcaaagcagacctcagacctcttactagtttcaccaaagactgacagaagcccttcctgtccagctttccccagctagcctgcccttttgagcaactctggggaaccatgattccctatcttccctttccttcacaggtctgcacacctcattgccccttttgcaactactgaggcacttgcagctgcctcagacttctcagctccccttgagatgcctggatcttcacacccccaactccttagctactaaggaatgtgcccctcacagggctgacctacccacagctgcctctcccacatgtgacccttacctacactctctggggacccccagtgttgcgcctttgtctctttgcctttgtccttaccctag

SEQ ID NO: 32 - DNA, 369 bp, synthetic construct
  source 1..369: mol_type = other DNA
gtgagtggagggcgggcacccccattccataccaggcctatcatctcctacatcggatggcttacatcactctacaccacgagggagcaggaaggtgttcagggtggaacctcggaagaggcacacccatccccttttgcaccatggaggcaggaagtgactaggtagcaacagaaaaccccaatgcctgaggctggactgcgatgcagaaaagcagggtcagtgcccagcagcatggctccaggcctagagagccagggcagagcctctgcaggagttatggggtgggtccgtgggtgggtgacttcttagatgagggtttcatgggaggtaccccgagggactctgaccatctgttcccacattcag

SEQ ID NO: 33 - DNA, 94 bp, synthetic construct
  source 1..94: mol_type = other DNA
gtgagggcaatggtgaggtctgggaagtgggctgaagacagcgttgggggccttggcaggatcacactctcagcttctcctccctgctccctag

SEQ ID NO: 34 - DNA, 133 bp, synthetic construct
  source 1..133: mol_type = other DNA
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 35 - DNA, 824 bp, synthetic construct
  source 1..824: mol_type = other DNA
gtaagtaccgcctatagactctataggcacacccctttggctcttatgcatgctatactgtttttggcttggggcctatacacccccgctccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagctcggagattgggctcgcaccgtgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcag

SEQ ID NO: 36 - DNA, 520 bp, synthetic construct
  source 1..520: mol_type = other DNA
tcctcactctcttccgcatcgctgtctgcgagggccagctgttgggctcgcggttgaggacaaactcttcgcggtctttccagtactcttggatcggaaacccgtcggcctccgaacggtactccgccaccgagggacctgagcgagtccgcatcgaccggatcggaaaacctctcgagaaaggcgtctaaccagtcacagtcgcaaggtaggctgagcaccgtggcgggcggcagcgggtggcggtcggggttgtttctggcggaggtgctgctgatgatgtaattaaagtaggcggtcttgagacggcggatggtcgaggtgaggtgtggcaggcttgagatccagctgttggggtgagtactccctctcaaaagcgggcattacttctgcgctaagattgtcagtttccaaaaacgaggaggatttgatattcacctggcccgatctggccatacacttgagtgacaatgacatccactttgcctttctctccacaggtgtccactcccaggtccaa

SEQ ID NO: 37 - DNA, 93 bp, synthetic construct
  source 1..93: mol_type = other DNA
ctctaaggtaaatataaaatttttaagtgtataatgtgttaaactactgattctaattgtttctctcttttagattccaacctttggaactga

SEQ ID NO: 38 - DNA, 99 bp, synthetic construct
  source 1..99: mol_type = other DNA
gtaagtttagtctttttgtcttttatttcaggtcccggatccggtggtggtgcaaatcaaagaactgctcctcagtggatgttgcctttacttctaggc

SEQ ID NO: 39 - DNA, 92 bp, synthetic construct
  source 1..92: mol_type = other DNA
aagaggtaagggtttaagggatggttggttggtggggtattaatgtttaattacctggagcacctgcctgaaatcactttttttcaggttgg

SEQ ID NO: 40 - DNA, 119 bp, synthetic construct
  source 1..119: mol_type = other DNA
gtacgatggcgcctccagctaaaagagctaaaagaggtaagggtttaagggatggttggttggtggggtattaatgtttaattacctgttttacaggcctgaaatcacttggttttagg

SEQ ID NO: 41 - DNA, 649 bp, synthetic construct
  source 1..649: mol_type = other DNA
ggatcgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcattttggcaaagaatt

SEQ ID NO: 42 - DNA, 296 bp, synthetic construct
  source 1..296: mol_type = other DNA
gaattaattcgctgtctgcgagggccggctgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaactgcaggtcg

SEQ ID NO: 43 - DNA, 573 bp, synthetic construct
  source 1..573: mol_type = other DNA
gtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacag

SEQ ID NO: 44 - DNA, 1979 bp, synthetic construct
  source 1..1979: mol_type = other DNA
atggagttctcaagtccttctcgcgaggtaagagagagagctcccaatcagcattgtcacagtgcttctggaatcctggcactggaatttaatgaatgacagactctctttgaatccagggccatcatggctctttgagcaaggcacagatggagggaggggtcgaagttgaaatgggtgggaagagtggtggggagcatcctgatttggggtgggcagagagttgtcatcagaagggttgcagggagagctgcacccaggtttctgtgggccttgtcctaatgaatgtgggagaccgggccatgggcacccaaaggcagctaagccctgcccaggagagtagttgaggggtggagaggggcttgcttttcagtcattcctcattctgtcctcaggaatgtcccaaaccactgagccgcgtgtcaattatggctggatctctgaccggactgctgctgctgcaggccgtgagttgggctagtggggcccggccctgcatcccaaagagctttggctacagctctgtggtgtgcgtgtgcaacgccacctactgcgacagtttcgaccctcccaccttcccagccctgggcacattctctcggtacgagtccactcgctcaggccggagaatggagctgtctatgggcccaatccaggctaatcataccggaacaggcctgctgctgacactgcagccagagcagaagtttcagaaggtgaaaggcttcgggggcgccatgaccgatgctgctgccctgaatatcctggccctgagtcccccagctcagaatctgctgctgaagtcatactttagtgaggaaggcattggatacaatattatccgggtgcctatggcctcctgtgacttctcaattcgcacctatacttacgctgatacccccgatgatttccagctgcacaacttcagtctgcccgaggaagataccaaactgaaaatcccactgatccatcgggctctgcagctggctcagcgcccagtgagcctgctggcctctccttggacctcaccaacatggctgaagactaacggcgccgtgaacgggaaaggcagtctgaaagggcagccaggcgacatctatcaccagacttgggcccggtacttcgtgaaatttctggatgcctacgctgagcacaaactgcagttttgggccgtgaccgccgaaaatgagccatcagccggcctgctgtctggctacccatttcagtgtctggggtttacaccagagcatcagagggacttcatcgcccgggacctgggaccaaccctggccaacagcacccaccataacgtgaggctgctgatgctggacgatcagcggctgctgctgcctcactgggctaaggtggtgctgaccgatccagaggctgccaagtacgtgcatggaatcgccgtgcactggtacctggatttcctggcccctgctaacgccaccctgggagagacccatcggctgttccccaacaccatgctgtttgcctctgaagcttgcgtggggtcaaagttttgggagcagtcagtgaggctgggcagttgggatcgggggatgcagtactcccacagtattatcaccaatctgctgtaccatgtggtgggctggaccgattggaatctggccctgaatccagaggggggccctaattgggtgcggaacttcgtggactccccaatcattgtggatattacaaaggacaccttctacaaacagcccatgttctaccacctgggccactttagtaagttcatccctgaggggtcacagcgggtgggcctggtggcctcccagaaaaacgacctggatgctgtggctctgatgcaccctgacggatcagccgtggtggtggtgctgaatcgctctagtaaggacgtgccactgaccatcaaggatccagctgtgggcttcctggagacaatcagtcccgggtactctatccacacctatctgtggcgccggcagtga

SEQ ID NO: 45 - DNA, 2163 bp, synthetic construct
  source 1..2163: mol_type = other DNA
atggagttctcaagtccttctcgcgaggaatgtcccaaaccactgagccgcgtgtcaattatggctggatctctgaccggactgctgctgctgcaggccgtgagttgggctagtggtgagtgagtcaaggcagtggggaggtagcacagagcctcccttctgcctcatagtcctttggtagccttccagtaagctggtggtagacttttagtaggtgctcaataaatccttttgagtgactgagaccaactttggggtgaggattttgttttttttcttttgaaacagagtcttactctgttgcctgggctggagtgcagtggtgcaattttggctcattccaacctctgcctcccagattcaagcgattctcttgcttcagcttcccaggtagctgggattacaggcggccaccactacgcccagctaatttttgtatttttagtagagacggggtttcaccatgctggcaaggcaggtctcaaactcctcacctcaggtgatccgcccacctcggcctcctaaagtgctaggattacaggtgtgagcccctgcgcccggccaaggggtgaggaattttgaaaccgtgttcagtctctcctagcagatgtgtccattctccatgtcttcatcagacctcactctgcttgtactccctccctcccaggggcccggccctgcatcccaaagagctttggctacagctctgtggtgtgcgtgtgcaacgccacctactgcgacagtttcgaccctcccaccttcccagccctgggcacattctctcggtacgagtccactcgctcaggccggagaatggagctgtctatgggcccaatccaggctaatcataccggaacaggcctgctgctgacactgcagccagagcagaagtttcagaaggtgaaaggcttcgggggcgccatgaccgatgctgctgccctgaatatcctggccctgagtcccccagctcagaatctgctgctgaagtcatactttagtgaggaaggcattggatacaatattatccgggtgcctatggcctcctgtgacttctcaattcgcacctatacttacgctgatacccccgatgatttccagctgcacaacttcagtctgcccgaggaagataccaaactgaaaatcccactgatccatcgggctctgcagctggctcagcgcccagtgagcctgctggcctctccttggacctcaccaacatggctgaagactaacggcgccgtgaacgggaaaggcagtctgaaagggcagccaggcgacatctatcaccagacttgggcccggtacttcgtgaaatttctggatgcctacgctgagcacaaactgcagttttgggccgtgaccgccgaaaatgagccatcagccggcctgctgtctggctacccatttcagtgtctggggtttacaccagagcatcagagggacttcatcgcccgggacctgggaccaaccctggccaacagcacccaccataacgtgaggctgctgatgctggacgatcagcggctgctgctgcctcactgggctaaggtggtgctgaccgatccagaggctgccaagtacgtgcatggaatcgccgtgcactggtacctggatttcctggcccctgctaacgccaccctgggagagacccatcggctgttccccaacaccatgctgtttgcctctgaagcttgcgtggggtcaaagttttgggagcagtcagtgaggctgggcagttgggatcgggggatgcagtactcccacagtattatcaccaatctgctgtaccatgtggtgggctggaccgattggaatctggccctgaatccagaggggggccctaattgggtgcggaacttcgtggactccccaatcattgtggatattacaaaggacaccttctacaaacagcccatgttctaccacctgggccactttagtaagttcatccctgaggggtcacagcgggtgggcctggtggcctcccagaaaaacgacctggatgctgtggctctgatgcaccctgacggatcagccgtggtggtggtgctgaatcgctctagtaaggacgtgccactgaccatcaaggatccagctgtgggcttcctggagacaatcagtcccgggtactctatccacacctatctgtggcgccggcagtga

SEQ ID NO: 46 - DNA, 1734 bp, synthetic construct
  source 1..1734: mol_type = other DNA
atggagttctcaagtccttctcgcgaggaatgtcccaaaccactgagccgcgtgtcaattatggctggatctctgaccggactgctgctgctgcaggccgtgagttgggctagtggggcccggccctgcatcccaaagagctttggctacagctctgtggtgtgcgtgtgcaacgccacctactgcgacagtttcgaccctcccaccttcccagccctgggcacattctctcggtacgagtccactcgctcaggccggagaatggagctgtctatgggcccaatccaggctaatcataccggaacaggtaaccattacacccctcaccccctgggccaggctgggtcctcctagaggtaaatggtgtcagtgatcaccatggagtttcccgctgggtactgatacccttattccctgtggatgtcctcaggcctgctgctgacactgcagccagagcagaagtttcagaaggtgaaaggcttcgggggcgccatgaccgatgctgctgccctgaatatcctggccctgagtcccccagctcagaatctgctgctgaagtcatactttagtgaggaaggcattggatacaatattatccgggtgcctatggcctcctgtgacttctcaattcgcacctatacttacgctgatacccccgatgatttccagctgcacaacttcagtctgcccgaggaagataccaaactgaaaatcccactgatccatcgggctctgcagctggctcagcgcccagtgagcctgctggcctctccttggacctcaccaacatggctgaagactaacggcgccgtgaacgggaaaggcagtctgaaagggcagccaggcgacatctatcaccagacttgggcccggtacttcgtgaaatttctggatgcctacgctgagcacaaactgcagttttgggccgtgaccgccgaaaatgagccatcagccggcctgctgtctggctacccatttcagtgtctggggtttacaccagagcatcagagggacttcatcgcccgggacctgggaccaaccctggccaacagcacccaccataacgtgaggctgctgatgctggacgatcagcggctgctgctgcctcactgggctaaggtggtgctgaccgatccagaggctgccaagtacgtgcatggaatcgccgtgcactggtacctggatttcctggcccctgctaacgccaccctgggagagacccatcggctgttccccaacaccatgctgtttgcctctgaagcttgcgtggggtcaaagttttgggagcagtcagtgaggctgggcagttgggatcgggggatgcagtactcccacagtattatcaccaatctgctgtaccatgtggtgggctggaccgattggaatctggccctgaatccagaggggggccctaattgggtgcggaacttcgtggactccccaatcattgtggatattacaaaggacaccttctacaaacagcccatgttctaccacctgggccactttagtaagttcatccctgaggggtcacagcgggtgggcctggtggcctcccagaaaaacgacctggatgctgtggctctgatgcaccctgacggatcagccgtggtggtggtgctgaatcgctctagtaaggacgtgccactgaccatcaaggatccagctgtgggcttcctggagacaatcagtcccgggtactctatccacacctatctgtggcgccggcagtga

SEQ ID NO: 47 - DNA, 2576 bp, synthetic construct
  source 1..2576: mol_type = other DNA
atggagttctcaagtccttctcgcgaggaatgtcccaaaccactgagccgcgtgtcaattatggctggatctctgaccggactgctgctgctgcaggccgtgagttgggctagtggggcccggccctgcatcccaaagagctttggctacagctctgtggtgtgcgtgtgcaacgccacctactgcgacagtttcgaccctcccaccttcccagccctgggcacattctctcggtacgagtccactcgctcaggccggagaatggagctgtctatgggcccaatccaggctaatcataccggaacaggcctgctgctgacactgcagccagagcagaagtttcagaaggtgaaaggcttcgggggcgccatgaccgatgctgctgccctgaatatcctggccctgagtcccccagctcagaatctgctgctgaagtcatactttagtgaggaaggtgaggaggaaggggacaagatgacatagagccattgaaacttttcgtttttcttttctttttttaaaatttttttgaggcagaatctcactctgcccattctgtcggcgagacaggagtgcagtggtgtgatctcccctcacagcaacctctgcctcccaggctatagtgattctcctgcctcagcctcctgagtagctggaattataggcgtgcgccactaccacctggctaatttttgtatttttagtagagacagggtttcatcatgttgaccaggctagtcttaaactcctgacctcaaatgatatacctgccttggcctcccgaagtgctggaattacaagtgtgagccaccgagcccagcagacacttttcttttttctttttttttttttgagacagagtctcgcactgtcacccaggctggagtgcagtggcacaatctcagctcactgcaacctccacctcccgggttcaggtgattctcctgtctcagcctctcgagtacctgggattacaggtgcctgccaccacgcccggctaattttttgtatttttagtagagacagggtttcactatgttggccaggatgattgcgaactcctgacctcgtgatctgcccacatcggcctcccaaagtgctgggattacatgcgtgagccactgacacttttctttgccctttctttggaccctgacttctgcccatccctgacatttggttcctgttttaatgccctgtgaaataagatttcaccgcctatcatctgctaactgctacggactcaggctcagaaaggcctgcgcttcacccaggtgccagcctccacaggttccaacccaggagcccaagttccctttggccctgactcagacactattaggactggcaagtgataagcagagtcccatactctcctattgactcggactaccatatcttgatcatccttttctgtaggcattggatacaatattatccgggtgcctatggcctcctgtgacttctcaattcgcacctatacttacgctgatacccccgatgatttccagctgcacaacttcagtctgcccgaggaagataccaaactgaaaatcccactgatccatcgggctctgcagctggctcagcgcccagtgagcctgctggcctctccttggacctcaccaacatggctgaagactaacggcgccgtgaacgggaaaggcagtctgaaagggcagccaggcgacatctatcaccagacttgggcccggtacttcgtgaaatttctggatgcctacgctgagcacaaactgcagttttgggccgtgaccgccgaaaatgagccatcagccggcctgctgtctggctacccatttcagtgtctggggtttacaccagagcatcagagggacttcatcgcccgggacctgggaccaaccctggccaacagcacccaccataacgtgaggctgctgatgctggacgatcagcggctgctgctgcctcactgggctaaggtggtgctgaccgatccagaggctgccaagtacgtgcatggaatcgccgtgcactggtacctggatttcctggcccctgctaacgccaccctgggagagacccatcggctgttccccaacaccatgctgtttgcctctgaagcttgcgtggggtcaaagttttgggagcagtcagtgaggctgggcagttgggatcgggggatgcagtactcccacagtattatcaccaatctgctgtaccatgtggtgggctggaccgattggaatctggccctgaatccagaggggggccctaattgggtgcggaacttcgtggactccccaatcattgtggatattacaaaggacaccttctacaaacagcccatgttctaccacctgggccactttagtaagttcatccctgaggggtcacagcgggtgggcctggtggcctcccagaaaaacgacctggatgctgtggctctgatgcaccctgacggatcagccgtggtggtggtgctgaatcgctctagtaaggacgtgccactgaccatcaaggatccagctgtgggcttcctggagacaatcagtcccgggtactctatccacacctatctgtggcgccggcagtga

SEQ ID NO: 48 - DNA, 1821 bp, synthetic construct
  source 1..1821: mol_type = other DNA
atggagttctcaagtccttctcgcgaggaatgtcccaaaccactgagccgcgtgtcaattatggctggatctctgaccggactgctgctgctgcaggccgtgagttgggctagtggggcccggccctgcatcccaaagagctttggctacagctctgtggtgtgcgtgtgcaacgccacctactgcgacagtttcgaccctcccaccttcccagccctgggcacattctctcggtacgagtccactcgctcaggccggagaatggagctgtctatgggcccaatccaggctaatcataccggaacaggcctgctgctgacactgcagccagagcagaagtttcagaaggtgaaaggcttcgggggcgccatgaccgatgctgctgccctgaatatcctggccctgagtcccccagctcagaatctgctgctgaagtcatactttagtgaggaaggcattggatacaatattatccgggtgcctatggcctcctgtgacttctcaattcgcacctatacttacgctgatacccccgatgatttccagctgcacaacttcagtctgcccgaggaagataccaaactgaaagtaggcattctagctttttcaggccctgagggccctgatgtctgggggttgagaaactgtagggtaggtctgcttgtacagacattttgtcccctgctgttttgtcctgggggtgggagggtggaggctaatggctgaaccggatgcactggttgggctagtatgtgttccaactctgggtgcttctctcttcactacctttgtctctagatcccactgatccatcgggctctgcagctggctcagcgcccagtgagcctgctggcctctccttggacctcaccaacatggctgaagactaacggcgccgtgaacgggaaaggcagtctgaaagggcagccaggcgacatctatcaccagacttgggcccggtacttcgtgaaatttctggatgcctacgctgagcacaaactgcagttttgggccgtgaccgccgaaaatgagccatcagccggcctgctgtctggctacccatttcagtgtctggggtttacaccagagcatcagagggacttcatcgcccgggacctgggaccaaccctggccaacagcacccaccataacgtgaggctgctgatgctggacgatcagcggctgctgctgcctcactgggctaaggtggtgctgaccgatccagaggctgccaagtacgtgcatggaatcgccgtgcactggtacctggatttcctggcccctgctaacgccaccctgggagagacccatcggctgttccccaacaccatgctgtttgcctctgaagcttgcgtggggtcaaagttttgggagcagtcagtgaggctgggcagttgggatcgggggatgcagtactcccacagtattatcaccaatctgctgtaccatgtggtgggctggaccgattggaatctggccctgaatccagaggggggccctaattgggtgcggaacttcgtggactccccaatcattgtggatattacaaaggacaccttctacaaacagcccatgttctaccacctgggccactttagtaagttcatccctgaggggtcacagcgggtgggcctggtggcctcccagaaaaacgacctggatgctgtggctctgatgcaccctgacggatcagccgtggtggtggtgctgaatcgctctagtaaggacgtgccactgaccatcaaggatccagctgtgggcttcctggagacaatcagtcccgggtactctatccacacctatctgtggcgccggcagtga

SEQ ID NO: 49 - DNA, 2166 bp, synthetic construct
  source 1..2166: mol_type = other DNA
atggagttctcaagtccttctcgcgaggaatgtcccaaaccactgagccgcgtgtcaattatggctggatctctgaccggactgctgctgctgcaggccgtgagttgggctagtggggcccggccctgcatcccaaagagctttggctacagctctgtggtgtgcgtgtgcaacgccacctactgcgacagtttcgaccctcccaccttcccagccctgggcacattctctcggtacgagtccactcgctcaggccggagaatggagctgtctatgggcccaatccaggctaatcataccggaacaggcctgctgctgacactgcagccagagcagaagtttcagaaggtgaaaggcttcgggggcgccatgaccgatgctgctgccctgaatatcctggccctgagtcccccagctcagaatctgctgctgaagtcatactttagtgaggaaggcattggatacaatattatccgggtgcctatggcctcctgtgacttctcaattcgcacctatacttacgctgatacccccgatgatttccagctgcacaacttcagtctgcccgaggaagataccaaactgaaaatcccactgatccatcgggctctgcagctggctcagcgcccagtgagcctgctggcctctccttggacctcaccaacatggctgaagactaacggcgccgtgaacgggaaaggcagtctgaaagggcagccaggcgacatctatcaccagacttgggcccggtacttcgtgaagtaagggatcagcaaggatgtgggatcaggactggcctcccatttagccatgctgatctgtgtcccaaccctcaacctagttccacttccagatctgcctgtcctcagctcacctttctaccttctgggcctttcagccttgggcctgtcaatcttgcccactccatcaggcttcctgttctctcggtctggcccactttctttttatttttcttcttttttttttttttgagaaggagtctctctctctgtcacccaggctggagtgctgtggcgccatcttcactcactgtaacctctgcctcctgagttcaagcaattctcctgcctcagccttccaagtagctgggattataggcgcctgccaccaggcccagctgatttttctatttttagtagagacggggtttcgccaggctgttctcgaactcctgaactcaagtgatccacctgcctcggcttcccaaagtgctgggattacaggtgtgagccaccacacccagctggtctggtccactttcttggccggatcattcatgacctttctcttgccagatttctggatgcctacgctgagcacaaactgcagttttgggccgtgaccgccgaaaatgagccatcagccggcctgctgtctggctacccatttcagtgtctggggtttacaccagagcatcagagggacttcatcgcccgggacctgggaccaaccctggccaacagcacccaccataacgtgaggctgctgatgctggacgatcagcggctgctgctgcctcactgggctaaggtggtgctgaccgatccagaggctgccaagtacgtgcatggaatcgccgtgcactggtacctggatttcctggcccctgctaacgccaccctgggagagacccatcggctgttccccaacaccatgctgtttgcctctgaagcttgcgtggggtcaaagttttgggagcagtcagtgaggctgggcagttgggatcgggggatgcagtactcccacagtattatcaccaatctgctgtaccatgtggtgggctggaccgattggaatctggccctgaatccagaggggggccctaattgggtgcggaacttcgtggactccccaatcattgtggatattacaaaggacaccttctacaaacagcccatgttctaccacctgggccactttagtaagttcatccctgaggggtcacagcgggtgggcctggtggcctcccagaaaaacgacctggatgctgtggctctgatgcaccctgacggatcagccgtggtggtggtgctgaatcgctctagtaaggacgtgccactgaccatcaaggatccagctgtgggcttcctggagacaatcagtcccgggtactctatccacacctatctgtggcgccggcagtga

SEQ ID NO: 50 - DNA, 2482 bp, synthetic construct
  source 1..2482: mol_type = other DNA
atggagttctcaagtccttctcgcgaggaatgtcccaaaccactgagccgcgtgtcaattatggctggatctctgaccggactgctgctgctgcaggccgtgagttgggctagtggggcccggccctgcatcccaaagagctttggctacagctctgtggtgtgcgtgtgcaacgccacctactgcgacagtttcgaccctcccaccttcccagccctgggcacattctctcggtacgagtccactcgctcaggccggagaatggagctgtctatgggcccaatccaggctaatcataccggaacaggcctgctgctgacactgcagccagagcagaagtttcagaaggtgaaaggcttcgggggcgccatgaccgatgctgctgccctgaatatcctggccctgagtcccccagctcagaatctgctgctgaagtcatactttagtgaggaaggcattggatacaatattatccgggtgcctatggcctcctgtgacttctcaattcgcacctatacttacgctgatacccccgatgatttccagctgcacaacttcagtctgcccgaggaagataccaaactgaaaatcccactgatccatcgggctctgcagctggctcagcgcccagtgagcctgctggcctctccttggacctcaccaacatggctgaagactaacggcgccgtgaacgggaaaggcagtctgaaagggcagccaggcgacatctatcaccagacttgggcccggtacttcgtgaaatttctggatgcctacgctgagcacaaactgcagttttgggccgtgaccgccgaaaatgagccatcagccggcctgctgtctggctacccatttcagtgtctggggtttacaccagagcatcagagggacttcatcgcccgggacctgggaccaaccctggccaacagcacccaccataacgtgaggctgctgatgctggacgatcagcggctgctgctgcctcactgggctaaggtggtaaggcctggacctccatggtgctccagtgaccttcaaatccagcatccaaatgactggctcccaaacttagagcgatttctctacccaactatggattcctagagcaccattcccctggacctccagggtgccatggatcccacagttgtcgcttgaaacctttctaggggctgggcgaggtggctcactcatgcaaacccagcactttgggaagccgaggcgggtgatcacctgaggtcaggagtttaagaccaccctggccaacgtgttgaaaccctgtgtctactaaaatacaaaaaaaaaaaattatctgggcatgatggtgggtgtctgtaatcccagctactcaggaggctgagaagggagaatcagttgaacccgggagatggtggttgcggtgagccgagatcgcgccactgcactccagcctgggaggctgagcgagactccatctcgaaacaaaacaaaacaaaactatctaggctgggggtggtggttcatgtatgtatgtgtatatacatatatatgtgtttatatgtatatatatatacacacacacacatacatacacacacatacacacacaaattagctgggtgtggcacccgtgtagtcccagctactcaggaggctaatgtgggaggatcagttgaccctaggaagtcaaggctgcagtgagtcgtgattgcgccactgtactccagcccgagtgacagagtgacatcctgtctcaaaaacaaaaaaaaatctccccaaacctctctagttgcattcttcccgtcacccaactccaggattcctacaacaggaactagaagttccagaagcctgtgtgcaaggtccaggatcagttgctcttcctttgcaggtgctgaccgatccagaggctgccaagtacgtgcatggaatcgccgtgcactggtacctggatttcctggcccctgctaacgccaccctgggagagacccatcggctgttccccaacaccatgctgtttgcctctgaagcttgcgtggggtcaaagttttgggagcagtcagtgaggctgggcagttgggatcgggggatgcagtactcccacagtattatcaccaatctgctgtaccatgtggtgggctggaccgattggaatctggccctgaatccagaggggggccctaattgggtgcggaacttcgtggactccccaatcattgtggatattacaaaggacaccttctacaaacagcccatgttctaccacctgggccactttagtaagttcatccctgaggggtcacagcgggtgggcctggtggcctcccagaaaaacgacctggatgctgtggctctgatgcaccctgacggatcagccgtggtggtggtgctgaatcgctctagtaaggacgtgccactgaccatcaaggatccagctgtgggcttcctggagacaatcagtcccgggtactctatccacacctatctgtggcgccggcagtga

SEQ ID NO: 51 - DNA, 2011 bp, synthetic construct
  source 1..2011: mol_type = other DNA
atggagttctcaagtccttctcgcgaggaatgtcccaaaccactgagccgcgtgtcaattatggctggatctctgaccggactgctgctgctgcaggccgtgagttgggctagtggggcccggccctgcatcccaaagagctttggctacagctctgtggtgtgcgtgtgcaacgccacctactgcgacagtttcgaccctcccaccttcccagccctgggcacattctctcggtacgagtccactcgctcaggccggagaatggagctgtctatgggcccaatccaggctaatcataccggaacaggcctgctgctgacactgcagccagagcagaagtttcagaaggtgaaaggcttcgggggcgccatgaccgatgctgctgccctgaatatcctggccctgagtcccccagctcagaatctgctgctgaagtcatactttagtgaggaaggcattggatacaatattatccgggtgcctatggcctcctgtgacttctcaattcgcacctatacttacgctgatacccccgatgatttccagctgcacaacttcagtctgcccgaggaagataccaaactgaaaatcccactgatccatcgggctctgcagctggctcagcgcccagtgagcctgctggcctctccttggacctcaccaacatggctgaagactaacggcgccgtgaacgggaaaggcagtctgaaagggcagccaggcgacatctatcaccagacttgggcccggtacttcgtgaaatttctggatgcctacgctgagcacaaactgcagttttgggccgtgaccgccgaaaatgagccatcagccggcctgctgtctggctacccatttcagtgtctggggtttacaccagagcatcagagggacttcatcgcccgggacctgggaccaaccctggccaacagcacccaccataacgtgaggctgctgatgctggacgatcagcggctgctgctgcctcactgggctaaggtggtgctgaccgatccagaggctgccaagtacgtgcatggaatcgccgtgcactggtacctggatttcctggcccctgctaacgccaccctgggagagacccatcggctgttccccaacaccatgctgtttgcctctgaagcttgcgtggggtcaaagttttgggagcagtcagtgaggctgggcagttgggatcgggggatgcagtactcccacagtattatcaccgtaagccaccccagtctcccttcctgcaaagcagacctcagacctcttactagtttcaccaaagactgacagaagcccttcctgtccagctttccccagctagcctgcccttttgagcaactctggggaaccatgattccctatcttccctttccttcacaggtctgcacacctcattgccccttttgcaactactgaggcacttgcagctgcctcagacttctcagctccccttgagatgcctggatcttcacacccccaactccttagctactaaggaatgtgcccctcacagggctgacctacccacagctgcctctcccacatgtgacccttacctacactctctggggacccccagtgttgcgcctttgtctctttgcctttgtccttaccctagaatctgctgtaccatgtggtgggctggaccgattggaatctggccctgaatccagaggggggccctaattgggtgcggaacttcgtggactccccaatcattgtggatattacaaaggacaccttctacaaacagcccatgttctaccacctgggccactttagtaagttcatccctgaggggtcacagcgggtgggcctggtggcctcccagaaaaacgacctggatgctgtggctctgatgcaccctgacggatcagccgtggtggtggtgctgaatcgctctagtaaggacgtgccactgaccatcaaggatccagctgtgggcttcctggagacaatcagtcccgggtactctatccacacctatctgtggcgccggcagtga

SEQ ID NO: 52 - DNA, 1980 bp, synthetic construct
  source 1..1980: mol_type = other DNA
atggagttctcaagtccttctcgcgaggaatgtcccaaaccactgagccgcgtgtcaattatggctggatctctgaccggactgctgctgctgcaggccgtgagttgggctagtggggcccggccctgcatcccaaagagctttggctacagctctgtggtgtgcgtgtgcaacgccacctactgcgacagtttcgaccctcccaccttcccagccctgggcacattctctcggtacgagtccactcgctcaggccggagaatggagctgtctatgggcccaatccaggctaatcataccggaacaggcctgctgctgacactgcagccagagcagaagtttcagaaggtgaaaggcttcgggggcgccatgaccgatgctgctgccctgaatatcctggccctgagtcccccagctcagaatctgctgctgaagtcatactttagtgaggaaggcattggatacaatattatccgggtgcctatggcctcctgtgacttctcaattcgcacctatacttacgctgatacccccgatgatttccagctgcacaacttcagtctgcccgaggaagataccaaactgaaaatcccactgatccatcgggctctgcagctggctcagcgcccagtgagcctgctggcctctccttggacctcaccaacatggctgaagactaacggcgccgtgaacgggaaaggcagtctgaaagggcagccaggcgacatctatcaccagacttgggcccggtacttcgtgaaatttctggatgcctacgctgagcacaaactgcagttttgggccgtgaccgccgaaaatgagccatcagccggcctgctgtctggctacccatttcagtgtctggggtttacaccagagcatcagagggacttcatcgcccgggacctgggaccaaccctggccaacagcacccaccataacgtgaggctgctgatgctggacgatcagcggctgctgctgcctcactgggctaaggtggtgctgaccgatccagaggctgccaagtacgtgcatggaatcgccgtgcactggtacctggatttcctggcccctgctaacgccaccctgggagagacccatcggctgttccccaacaccatgctgtttgcctctgaagcttgcgtggggtcaaagttttgggagcagtcagtgaggctgggcagttgggatcgggggatgcagtactcccacagtattatcaccaatctgctgtaccatgtggtgggctggaccgattggaatctggccctgaatccagaggggggccctaattgggtgcggaacttcgtggactccccaatcattgtggatattacaaaggacaccttctacaaacagcccatgttctaccacctgggccactttaggtgagtggagggcgggcacccccattccataccaggcctatcatctcctacatcggatggcttacatcactctacaccacgagggagcaggaaggtgttcagggtggaacctcggaagaggcacacccatccccttttgcaccatggaggcaggaagtgactaggtagcaacagaaaaccccaatgcctgaggctggactgcgatgcagaaaagcagggtcagtgcccagcagcatggctccaggcctagagagccagggcagagcctctgcaggagttatggggtgggtccgtgggtgggtgacttcttagatgagggtttcatgggaggtaccccgagggactctgaccatctgttcccacattcagtaagttcatccctgaggggtcacagcgggtgggcctggtggcctcccagaaaaacgacctggatgctgtggctctgatgcaccctgacggatcagccgtggtggtggtgctgaatcgctctagtaaggacgtgccactgaccatcaaggatccagctgtgggcttcctggagacaatcagtcccgggtactctatccacacctatctgtggcgccggcagtga

SEQ ID NO: 53 - DNA, 1705 bp, synthetic construct
  source 1..1705: mol_type = other DNA
atggagttctcaagtccttctcgcgaggaatgtcccaaaccactgagccgcgtgtcaattatggctggatctctgaccggactgctgctgctgcaggccgtgagttgggctagtggggcccggccctgcatcccaaagagctttggctacagctctgtggtgtgcgtgtgcaacgccacctactgcgacagtttcgaccctcccaccttcccagccctgggcacattctctcggtacgagtccactcgctcaggccggagaatggagctgtctatgggcccaatccaggctaatcataccggaacaggcctgctgctgacactgcagccagagcagaagtttcagaaggtgaaaggcttcgggggcgccatgaccgatgctgctgccctgaatatcctggccctgagtcccccagctcagaatctgctgctgaagtcatactttagtgaggaaggcattggatacaatattatccgggtgcctatggcctcctgtgacttctcaattcgcacctatacttacgctgatacccccgatgatttccagctgcacaacttcagtctgcccgaggaagataccaaactgaaaatcccactgatccatcgggctctgcagctggctcagcgcccagtgagcctgctggcctctccttggacctcaccaacatggctgaagactaacggcgccgtgaacgggaaaggcagtctgaaagggcagccaggcgacatctatcaccagacttgggcccggtacttcgtgaaatttctggatgcctacgctgagcacaaactgcagttttgggccgtgaccgccgaaaatgagccatcagccggcctgctgtctggctacccatttcagtgtctggggtttacaccagagcatcagagggacttcatcgcccgggacctgggaccaaccctggccaacagcacccaccataacgtgaggctgctgatgctggacgatcagcggctgctgctgcctcactgggctaaggtggtgctgaccgatccagaggctgccaagtacgtgcatggaatcgccgtgcactggtacctggatttcctggcccctgctaacgccaccctgggagagacccatcggctgttccccaacaccatgctgtttgcctctgaagcttgcgtggggtcaaagttttgggagcagtcagtgaggctgggcagttgggatcgggggatgcagtactcccacagtattatcaccaatctgctgtaccatgtggtgggctggaccgattggaatctggccctgaatccagaggggggccctaattgggtgcggaacttcgtggactccccaatcattgtggatattacaaaggacaccttctacaaacagcccatgttctaccacctgggccactttagtaagttcatccctgaggggtcacagcgggtgggcctggtggcctcccagaaaaacgacctggatgctgtggctctgatgcaccctgacggatcagccgtggtggtggtgctgaatcggtgagggcaatggtgaggtctgggaagtgggctgaagacagcgttgggggccttggcaggatcacactctcagcttctcctccctgctccctagctctagtaaggacgtgccactgaccatcaaggatccagctgtgggcttcctggagacaatcagtcccgggtactctatccacacctatctgtggcgccggcagtga

SEQ ID NO: 54 - DNA, 2189 bp, synthetic construct
  source 1..2189: mol_type = other DNA
atggagttctcaagtccttctcgcgaggtaagagagagagctcccaatcagcattgtcacagtgcttctggaatcctggcactggaatttaatgaatgacagactctctttgaatccagggccatcatggctctttgagcaaggcacagatggagggaggggtcgaagttgaaatgggtgggaagagtggtggggagcatcctgatttggggtgggcagagagttgtcatcagaagggttgcagggagagctgcacccaggtttctgtgggccttgtcctaatgaatgtgggagaccgggccatgggcacccaaaggcagctaagccctgcccaggagagtagttgaggggtggagaggggcttgcttttcagtcattcctcattctgtcctcaggaatgtcccaaaccactgagccgcgtgtcaattatggctggatctctgaccggactgctgctgctgcaggccgtgagttgggctagtggggcccggccctgcatcccaaagagctttggctacagctctgtggtgtgcgtgtgcaacgccacctactgcgacagtttcgaccctcccaccttcccagccctgggcacattctctcggtacgagtccactcgctcaggccggagaatggagctgtctatgggcccaatccaggctaatcataccggaacaggcctgctgctgacactgcagccagagcagaagtttcagaaggtgaaaggcttcgggggcgccatgaccgatgctgctgccctgaatatcctggccctgagtcccccagctcagaatctgctgctgaagtcatactttagtgaggaaggcattggatacaatattatccgggtgcctatggcctcctgtgacttctcaattcgcacctatacttacgctgatacccccgatgatttccagctgcacaacttcagtctgcccgaggaagataccaaactgaaagtaggcattctagctttttcaggccctgagggccctgatgtctgggggttgagaaactgtagggtaggtctgcttgtacagacattttgtcccctgctgttttgtcctgggggtgggagggtggaggctaatggctgaaccggatgcactggttgggctagtatgtgttccaactctgggtgcttctctcttcactacctttgtctctagatcccactgatccatcgggctctgcagctggctcagcgcccagtgagcctgctggcctctccttggacctcaccaacatggctgaagactaacggcgccgtgaacgggaaaggcagtctgaaagggcagccaggcgacatctatcaccagacttgggcccggtacttcgtgaaatttctggatgcctacgctgagcacaaactgcagttttgggccgtgaccgccgaaaatgagccatcagccggcctgctgtctggctacccatttcagtgtctggggtttacaccagagcatcagagggacttcatcgcccgggacctgggaccaaccctggccaacagcacccaccataacgtgaggctgctgatgctggacgatcagcggctgctgctgcctcactgggctaaggtggtgctgaccgatccagaggctgccaagtacgtgcatggaatcgccgtgcactggtacctggatttcctggcccctgctaacgccaccctgggagagacccatcggctgttccccaacaccatgctgtttgcctctgaagcttgcgtggggtcaaagttttgggagcagtcagtgaggctgggcagttgggatcgggggatgcagtactcccacagtattatcaccaatctgctgtaccatgtggtgggctggaccgattggaatctggccctgaatccagaggggggccctaattgggtgcggaacttcgtggactccccaatcattgtggatattacaaaggacaccttctacaaacagcccatgttctaccacctgggccactttagtaagttcatccctgaggggtcacagcgggtgggcctggtggcctcccagaaaaacgacctggatgctgtggctctgatgcaccctgacggatcagccgtggtggtggtgctgaatcgctctagtaaggacgtgccactgaccatcaaggatccagctgtgggcttcctggagacaatcagtcccgggtactctatccacacctatctgtggcgccggcagtga

SEQ ID NO: 55 - DNA, 1750 bp, synthetic construct
  source 1..1750: mol_type = other DNA
gtaagtatcaaggttacaagacaggtttaaggagaccaatagaaactgggcttgtcgagacagagaagactcttgcgtttctgataggcacctattggtcttactgacatccactttgcctttctctccacaggccaccatggagttctcaagtccttctcgcgaggaatgtcccaaaccactgagccgcgtgtcaattatggctggatctctgaccggactgctgctgctgcaggccgtgagttgggctagtggggcccggccctgcatcccaaagagctttggctacagctctgtggtgtgcgtgtgcaacgccacctactgcgacagtttcgaccctcccaccttcccagccctgggcacattctctcggtacgagtccactcgctcaggccggagaatggagctgtctatgggcccaatccaggctaatcataccggaacaggcctgctgctgacactgcagccagagcagaagtttcagaaggtgaaaggcttcgggggcgccatgaccgatgctgctgccctgaatatcctggccctgagtcccccagctcagaatctgctgctgaagtcatactttagtgaggaaggcattggatacaatattatccgggtgcctatggcctcctgtgacttctcaattcgcacctatacttacgctgatacccccgatgatttccagctgcacaacttcagtctgcccgaggaagataccaaactgaaaatcccactgatccatcgggctctgcagctggctcagcgcccagtgagcctgctggcctctccttggacctcaccaacatggctgaagactaacggcgccgtgaacgggaaaggcagtctgaaagggcagccaggcgacatctatcaccagacttgggcccggtacttcgtgaaatttctggatgcctacgctgagcacaaactgcagttttgggccgtgaccgccgaaaatgagccatcagccggcctgctgtctggctacccatttcagtgtctggggtttacaccagagcatcagagggacttcatcgcccgggacctgggaccaaccctggccaacagcacccaccataacgtgaggctgctgatgctggacgatcagcggctgctgctgcctcactgggctaaggtggtgctgaccgatccagaggctgccaagtacgtgcatggaatcgccgtgcactggtacctggatttcctggcccctgctaacgccaccctgggagagacccatcggctgttccccaacaccatgctgtttgcctctgaagcttgcgtggggtcaaagttttgggagcagtcagtgaggctgggcagttgggatcgggggatgcagtactcccacagtattatcaccaatctgctgtaccatgtggtgggctggaccgattggaatctggccctgaatccagaggggggccctaattgggtgcggaacttcgtggactccccaatcattgtggatattacaaaggacaccttctacaaacagcccatgttctaccacctgggccactttagtaagttcatccctgaggggtcacagcgggtgggcctggtggcctcccagaaaaacgacctggatgctgtggctctgatgcaccctgacggatcagccgtggtggtggtgctgaatcgctctagtaaggacgtgccactgaccatcaaggatccagctgtgggcttcctggagacaatcagtcccgggtactctatccacacctatctgtggcgccggcagtga

SEQ ID NO: 56 - DNA, 1653 bp, synthetic construct
  source 1..1653: mol_type = other DNA
atggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaa

(19) United States
(12) Patent Application Publication
(10) Pub. No.: US 2026/0347875 A1
(43) Pub. Date: Nov. 5, 2026

(54) CONSTRUCT FOR ENHANCING GENE EXPRESSION

(71) Applicant: LINGYI BIOTECH CO., LTD., Shanghai (CN)

(72) Inventors: Qing LIN, Shanghai (CN); Yixiong CHEN, Shanghai (CN); Xiaojing SHENG, Shanghai (CN); Tianqi JIANG, Shanghai (CN); Qiang WU, Shanghai (CN)

(73) Assignee: LINGYI BIOTECH CO., LTD., Shanghai (CN)

(21) Appl. No.: 19/119,353

(22) PCT Filed: Oct. 7, 2023
(86) PCT No.: PCT/CN2023/123282

§ 371 Date: Apr. 8, 2025

(30) Foreign Application Priority Data
Oct. 8, 2022 (WO) PCT/CN2022/123892

(51) Int. Cl. C12N 15/86

(52) U.S. Cl. CPC C12N 15/86; C12N 2750/14143; C12N 2830/42

(57) ABSTRACT

The present disclosure relates to an expression construct, vectors, viral particles, or compositions. The expression construct comprises a transcription regulatory element operably linked to a polynucleotide sequence of interest, wherein the expression construct comprises a promoter, an enhancer and optional intron, the enhancer is upstream of the promoter. The invention further relates uses of expression construct, vectors, viral particles, or compositions.

### PRIORITY

This application claims the benefit of, and priority to, PCT Application No. PCT/CN2022/123892, filed Oct. 8, 2022, the entire contents of which are hereby incorporated by reference.

### FIELD OF THE INVENTION

The present disclosure relates to a nucleotide construct. The invention further relates to said nucleotide construct, an expression vector, and its use.

### BACKGROUND

There is still an unmet need in the art for alternative and preferably improved methods for regulating the transcription of a transcript and optionally regulating the expression of a protein or polypeptide of interest.

### SUMMARY OF THE INVENTION

The present disclosure an expression construct comprising a transcription regulatory element operably linked to a polynucleotide sequence of interest, wherein the expression construct comprises a promoter, and an enhancer, the enhancer is upstream of the promoter,
wherein the expression construct comprises the elements, in a 5′ to 3′ direction:
a) Enhance 3, the Enhance 3 is optional;
b) Enhance 2;
c) Enhance 1; and
d) promoter;
wherein the Enhance 1 comprises all or a portion of a sequence comprising at least one selected from the sequence consisting of SEQ ID NOs: 13 and 15, and at least 80%, at least 85%, at least 90%, at least 95%, at least 99%, at least 99.5%, at least 99.8% identical to SEQ ID NOs: 13 and 15, the all or the portion of the sequence retains the functionality of enhancer, and
the Enhance 2 comprises all or a portion of a sequence comprising at least one selected from the sequence consisting of SEQ ID NOs: 8-11 and 13, and at least 80%, at least 85%, at least 90%, at least 95%, at least 99%, at least 99.5%, at least 99.8% identical to SEQ ID NOs: 8-11 and 13, the all or the portion of the sequence retains the functionality of enhancer.

In some embodiments, the Enhance 3 comprises all or a portion of a sequence comprising at least one selected from the sequence consisting of SEQ ID NOs: 8-11, and at least 80%, at least 85%, at least 90%, at least 95%, at least 99%, at least 99.5%, at least 99.8% identical to SEQ ID NOs: 8-11, the all or the portion of the sequence retains the functionality of enhancer.

In some embodiments, the promoter comprises all or a portion of a sequence selected from the sequence consisting of SEQ ID NOs: 2-6, and at least 80%, at least 85%, at least 90%, at least 95%, at least 99%, at least 99.5%, at least 99.8% identical to SEQ ID NOs: 2-6, the all or the portion of the sequence retains the functionality of promoter; In some embodiments, the promoter comprises all or a portion of a sequence selected from the sequence consisting of SEQ ID NOs: 3-6, and at least 80%, at least 85%, at least 90%, at least 95%, at least 99%, at least 99.5%, at least 99.8% identical to SEQ ID NOs: 3-6, the all or the portion of the sequence retains the functionality of promoter.

In some embodiments, the Enhance 1 comprises all or a portion of a sequence comprising at least one selected from the sequence consisting of SEQ ID NOs: 13 and 15, and at least 80%, at least 85%, at least 90%, at least 95%, at least 99%, at least 99.5%, at least 99.8% identical to SEQ ID NOs: 13 and 15, the all or the portion of the sequence retains the functionality of enhancer; the Enhance 2 comprises all or a portion of a sequence comprising at least one selected from the sequence consisting of SEQ ID NOs: 8-9, and at least 80%, at least 85%, at least 90%, at least 95%, at least 99%, at least 99.5%, at least 99.8% identical to SEQ ID NOs: 8-9, the all or the portion of the sequence retains the functionality of enhancer.

In some embodiments, the Enhance 1 comprises all or a portion of a sequence comprising at least one selected from the sequence consisting of SEQ ID NOs: 13 and 15, and at least 80%, at least 85%, at least 90%, at least 95%, at least 99%, at least 99.5%, at least 99.8% identical to SEQ ID NOs: 13 and 15, the all or the portion of the sequence retains the functionality of enhancer; the Enhance 2 comprises all or a portion of a sequence comprising SEQ ID NO: 8, and at least 80%, at least 85%, at least 90%, at least 95%, at least 99%, at least 99.5%, at least 99.8% identical to SEQ ID NOs: 8, the all or the portion of the sequence retains the functionality of enhancer.

In some embodiments, the expression construct further comprises an untranslated intron region.

In some embodiments, the untranslated intron region comprises all or a portion of a sequence selected from the sequence consisting of SEQ ID NOs: 24-43, and at least 80%, at least 85%, at least 90%, at least 95%, at least 99%, at least 99.5%, at least 99.8% identical to SEQ ID NOs: 24-43.

In some embodiments, the untranslated intron region is operably linked to 5′ terminal of the polynucleotide sequence of interest.

In some embodiments, the untranslated intron region is located between 5′ and 3′ terminal of the polynucleotide sequence of interest.

In another aspect, the disclosure provides a vector comprising the expression construct of present disclosure. The vector is viral vector, preferably AAV vector. The vector further comprises two adeno-associated virus inverted terminal repeats (ITR) sequences flanking the expression construct, preferably further comprises a poly A sequence.

In another aspect, the disclosure provides an adeno-associated virus (AAV) comprising the vector of present disclosure and capsid protein.

In some embodiments, the AAV is selected from the group consisting of: serotype AAV1, AAV2, AAV3, AAV3B, AAV4, AAV5, AAV6, AAV7, AAV8, AAV9, AAV10, AAV11, AAV12, AAV13, AAVrh10, AAVhu37 or any one of the AAV serotypes isolated from human and nonhuman mammalians or variant thereof.

In additional aspect, the disclosure provides a composition comprising the expression construct, vector, or AAV of present disclosure and a pharmaceutically acceptable excipient.

In further aspect, the disclosure provides the expression construct, vector, or AAV of present disclosure for use in a method of treatment.

In some embodiments, the disclosure provides use of the expression construct, vector, or AAV of present disclosure in the preparation of a medication for treatment of a disease or condition in a subject.

In some embodiments, the disclosure provides the expression construct, vector, or AAV of present disclosure for use in a method for treating a disease or condition in a subject.

In some embodiments, the disclosure provides a method for treating a disease or condition in a subject, comprising administering an effective amount of the expression construct, vector, or AAV of present disclosure to the patient.

In further aspect, the disclosure provides the expression construct, vector, or AAV of present disclosure for use in a method of expressing the nucleotide sequence of interest in a subject.

In further aspect, the disclosure provides the expression construct, vector, or AAV of present disclosure for use in a method of expressing the nucleotide sequence of interest in a subject.

### BRIEF DESCRIPTION OF THE FIGURES

FIG. 1 showed luciferase and GCase activity in HepG2 Cells with chimeric HSRE constructs. (A) Luciferase activity comparisons for different CHSREs based on promoter HSRE002. P<0.05 for Group PG135 and P<0.01 for Group PG139, PG140, PG131, PG132, PG141, PG142, PG143 compared with PG130. (B) Luciferase activity comparisons for different CHSREs based on promoter HSRE005. P<0.05 for Group PG148 and PG151 and P<0.01 for PG146, PG149, PG147 and PG150 compared with PG145. (C) Luciferase activity comparisons for different CHSREs based on promoter HSRE004. P<0.01 for all groups compared with PG152. (D) Luciferase activity comparisons for different CHSREs based on promoter HSRE003. P<0.01 for Group PG165 and P<0.01 for Group PG163, PG160, PG161, PG162, PG164 compared with PG159. (E) GCase enzyme activity comparisons for different CHSREs based on promoter HSRE002. P<0.05 for Group PG025 and P<0.01 for Group PG023, PG026, PG028, PG029, PG030, PG031 compared with PG022. (F) GCase enzyme activity comparisons for different CHSREs based on promoter HSRE005. P<0.05 for Group PG035 and P<0.01 for other Groups compared with PG032. (G) GCase enzyme activity comparisons for different CHSREs based on promoter HSRE004. P<0.05 for Group PG042 and P<0.01 for Group PG040, PG041, PG043, PG044 and PG045 compared with PG039. (H) GCase enzyme activity comparisons for different CHSREs based on promoter HSRE003. P<0.05 for Group PG049 and PG052 and P<0.01 for Group PG047, PG048 and PG051 compared with PG046. Values were shown as the mean±SEM. N=3 for each experimental group.

FIG. 2 showed luciferase and GCase activities in HSRE012 constructs in HepG2. (A) Luciferase activity in constructs with various HSRE012 locations flanked by promoter HSRE002. Significant differences observed: PG133 vs. PG131 (P<0.01), PG134 vs. PG132 (P<0.01). (B) Luciferase activity comparisons for different HSRE012 copy numbers flanked by promoter HSRE002. No significant differences detected: PG136 vs. PG131, PG137 vs. PG132, PG138 vs. PG135 (Values: mean±SEM, N=3 per group). (C) GCase enzyme activities in constructs with different HSRE012 locations flanked by promoter HSRE002. Significant differences found: PG053 vs. PG025 (P<0.01), PG054 vs. PG026 (P<0.01). (D) GCase enzyme activity comparisons for various HSRE012 copy numbers flanked by promoter HSRE002. No significant differences observed: PG055 vs. PG025, PG056 vs. PG026, PG057 vs. PG027 (Values: mean SEM, N=3 per group).

FIG. 3 showed GCase Enzyme activities in constructs with exogenous introns flanked by HSREs in vitro. (A) GCase enzyme activities of construct with endogenous intron in Huh7 cells. P<0.05 for Group PG059 compared with PG058. (B) GCase enzyme activities of different constructs in HepG2 cells in vitro. P<0.01 for Group PG076, PG078 and PG081 compared with PG074. P<0.01 for Group PG090 compared with PG082 and P<0.05 for Group PG091 compared with PG082. P<0.01 for Group PG093, PG094, PG095, PG097, PG098, PG099, PG100 and PG101 compared with PG092. (C) GCase enzyme activities of different constructs in HEK293T cells. Error bars represented mean±SEM. N=3 for each experimental group.

FIG. 4 showed GCase enzyme activities in constructs with various introns flanked by Cchimeric HSREs in vitro. (A) GCase enzyme activities of different constructs in HepG2 cells. P<0.05 for Group PG107 and PG108 compared with PG168. P<0.01 for Group PG114 and PG115 compared with PG169. (B) GCase enzyme activities of different constructs in HEK293T cells. Error bars represented mean±SEM. N=3 for each experimental group.

FIG. 5 showed GCase enzyme activities in serum and tissue lysates 2 weeks post-injection of AAV8 vectors at a dose of 2E12 vg/kg in wild-type mice. (A) GCase enzyme activities were measured in serum. P<0.05 for Group PG026, PG037, PG051 and PG105 and P<0.01 for Group PG103, PG104 and PG105 compared with PG127. P<0.01 for Group PG103 and PG104 compared with PG102. P<0.01 for Group PG103 and PG104 compared with PG026. (B) GCase enzyme activities were measured in liver lysates. P<0.05 for Group PG026 and P<0.01 for Group PG103, PG104 and PG105 compared with PG127. P<0.01 for Group PG103 and PG104 compared with PG102. P<0.01 for Group PG103 and PG104 compared with PG026. P<0.01 for Group PG105 compared with PG051. (C) GCase enzyme activity measured in lung lysates. P<0.05 for Group PG105 and PG026 and P<0.01 for Group PG103 and PG104 compared with PG127. P<0.05 for Group PG104 and P<0.01 for Group PG103 compared with PG102. P<0.05 for Group PG103 compared with PG026. (D) GCase enzyme activity were measured in spleen lysates. P<0.01 for Group PG026, PG103, PG104 and PG105 compared with PG127. P<0.01 for Group PG103 and PG104 compared with PG102. P<0.01 for Group PG103 compared with PG026. Values are shown as the mean±SEM. N=4 for each experimental group.

FIG. 6 showed markable efficacy of AAV8 gene therapy candidates regulated by chimeric HSREs in Gaucher mice. (A) GCase enzyme activities were measured in serum 8 weeks post-injection of AAV8 candidates at dose of 2E12 vg/kg. P<0.05 for Group PG118, PG119, PG120 and PG122 compared with Buffer control or Cerezyme groups. P<0.05 for Group PG118 and PG122 and P<0.01 for Group PG119 and PG120 compared with PG127. (B) Substrate accumulation was measured in serum 8 weeks post-injection of AAV8 candidates at dose of 2E12 vg/kg. P<0.01 for all groups compared with Buffer control or Cerezyme groups. P<0.05 for Group PG119 and P<0.01 for Group PG118, PG120 and PG122 compared with PG127. Serum glucosylsphingosine level in PG119 was below the detection limit. Naive group represented the corresponding wild type mice. Values were shown as the mean±SEM. N=5 for each experimental group.

FIG. 7 showed GCase enzyme activities in tissue lysates from Gaucher mice 12 weeks post-injection of AAV8 candidates at a dose of 2E12 vg/kg. (A) GCase enzyme activities were measured in liver lysates. P<0.01 for Group PG118-PG122 compared with Buffer control or Cerezyme groups. P<0.05 for Group PG120 and PG122 and P<0.01 for Group PG118 and PG119 compared with PG127. (B) GCase enzyme activities measured in lung lysates. P<0.05 for Group PG118-PG122 compared with Buffer control or Cerezyme groups. P<0.05 for Group PG118 and PG122 and P<0.01 for Group PG119 and PG120 compared with PG127. (C) GCase enzyme activity measured in spleen lysates. P<0.01 for Group PG118-PG122 compared with Buffer control group. P<0.05 for Group PG120 and P<0.01 for Group PG118, PG119 and PG122 compared with Cerezyme group. P<0.05 for Group PG118 and PG122 and P<0.01 for Group PG119 and PG120 compared with PG127. Naive group represented the wild type mice. Values were shown as the mean±SEM. N=5 for each experimental group.

FIG. 8 showed substrate accumulation of glucosylsphingosine in tissue lysates 12 weeks post-injection of AAV8 candidates at a dose of 2E12 vg/kg in Gaucher mice. (A) Substrate accumulation was measured in liver lysates. P<0.01 for Group PG001, PG011, PG119 and PG120 compared with Buffer control or Cerezyme groups. P<0.01 for groups of PG119 and PG120 compared with PG127. (B) Substrate accumulation measured in lung lysate. P<0.05 for Group PG001 and P<0.01 for Group PG011, PG119 and PG120 compared with Buffer control or Cerezyme groups. P<0.05 for groups of PG119 and PG120 compared with PG127. (C) Substrate accumulation measured in spleen lysate. P<0.01 for Group PG001, PG011, PG119 and PG120 compared with Buffer control or Cerezyme groups. P<0.01 for groups of PG119 and PG120 compared with PG127. Naive represented wild type mice. Error bars represent mean±SEM. N=5 for each experimental group. LC-MS/MS was used to analyze glucosylsphingosine level in different tissue lysates.

FIG. 9 showed GCase enzyme activities in serum and tissue lysates 2 weeks post-injection of AAV9 candidates at a dose of 2E12 vg/kg in wild-type mice. (A) GCase enzyme activities were measured in serum. P<0.05 for Group PG124 and P<0.01 for Group PG123, PG125 and PG126 compared with Buffer control group. P<0.05 for Group PG124 and PG125 and P<0.01 for Group PG123 and PG126 compared with PG001. P<0.05 for Group PG125 and P<0.01 for Group PG123 and PG126 compared with PG128. (B) GCase enzyme activities were measured in liver lysates. P<0.01 for Group PG123-PG126 compared with Buffer control group. P<0.05 for Group PG124 and PG125 and P<0.01 for Group PG123 and PG126 compared with PG001. P<0.05 for Group PG124 and PG125 and P<0.01 for Group PG123 and PG126 compared with PG128. (C) GCase enzyme activities were measured in lung lysates. P<0.05 for Group PG124 and P<0.01 for Group PG123, PG125 and PG126 compared with Buffer control group. P<0.05 for Group PG124 and P<0.01 for Group PG123, PG125 and PG126 compared with PG001. P<0.05 for Group PG123 and PG125 and P<0.01 for Group PG126 compared with PG128. (D) GCase enzyme activities were measured in spleen lysates. P<0.01 for Group PG123-PG126 compared with Buffer control group. P<0.05 for Group PG124 and P<0.01 for Group PG123, PG125 and PG126 compared with PG001. P<0.05 for Group PG124 and P<0.01 for Group PG123, PG125 and PG126 compared with PG128. Values were shown as the mean±SEM. N=4 for each experimental group.

### DETAILED DESCRIPTION

Embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the invention to those skilled in the art. The terminology used in the description herein is for the purpose of describing embodiments only and is not intended to be limiting.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein.

### Definitions

As used in the description of the invention and the appended claims, the singular forms “a,” “an” and “the” are intended to include the plural forms as well, unless the context clearly indicates otherwise.

As used herein, the term “comprising” is intended to mean that the compositions and methods include the recited elements, but do not exclude others.

As used herein, the terms “nucleotide” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising, consisting essentially of, or consisting of purine and pyrimidine bases or other natural, chemically, or biochemically modified, non-natural, or derivatized nucleotide bases.

As used herein, “expression” refers to the two-step process by which polynucleotides are transcribed into mRNA and/or the process by which the transcribed mRNA is subsequently translated into peptides, polypeptides, or proteins. If the polynucleotide is derived from genomic DNA, expression may include splicing of the mRNA in a eukaryotic cell.

The term “encodes” or “encoding” as it is applied to polynucleotides refers to a polynucleotide which is said to “encode” a polypeptide if it can be transcribed to produce the mRNA for the polypeptide and/or a fragment thereof. The antisense strand is the complement of such a nucleic acid, and the encoding sequence can be deduced therefrom.

The term “promoter” as used herein means a control sequence that is a region of a polynucleotide sequence at winch the initiation and rate of transcription of a coding sequence, such as a gene or a transgene, are controlled. Promoters may be constitutive, inducible, repressible, or tissue-specific. In embodiments, the promoter is used together with an enhancer to increase the transcription efficiency. An enhancer is a regulatory element that increases the expression of a target sequence.

The term “protein”, “peptide” and “polypeptide” are used interchangeably and in their broadest sense to refer to a compound of two or more subunits of amino acids, amino acid analogs or peptidomimetics. The subunits may be linked by peptide bonds. In another aspect, the subunit may be linked by other bonds, e.g., ester, ether, etc. A protein or peptide must contain at least, two amino acids and no limitation is placed on the maximum number of amino acids which may comprise, consist essentially of or consist of a protein's or peptide's sequence. As used herein the term “amino acid” refers to either natural and/or unnatural or synthetic amino acids, including glycine and both the D and L optical isomers, amino acid analogs and peptidomimetics.

“Identical” refers to sequence similarity between two peptides or between two nucleic acid molecules. Percent identity can be determined by comparing a position in each sequence that may be aligned for purposes of comparison. When a position in the compared sequence is occupied by the same base or amino acid, then the molecules are identical at that position. A degree of identity between sequences is a function of the number of matching positions shared by the sequences.

As used herein, the term “vector” refers to a nucleic acid comprising, consisting essentially of, or consisting of an intact replicon such that the vector may be replicated when placed within a cell, for example by a process of transfection, infection, or transformation. It is understood in the art that once inside a cell, a vector may replicate as an extrachromosomal (episome) element or may be integrated into a host cell chromosome. Vectors may include nucleic acids derived from retroviruses, adenoviruses, herpesviruses, baculoviruses, modified baculoviruses, papovaviruses, AAV viral vectors, lentiviral vectors, adenovirus vectors, alphavirus vectors and the like. Alphavirus vectors, such as Semliki Forest virus-based vectors and Sindbis virus-based vectors, have also been developed for use in gene therapy and immunotherapy. See, e.g., Schlesinger and Dubensky (1999) Curr. Opin. Biotechnol. 5:434-439 and Ying, et al. (1999) Nat. Med. 5(7):823-827.

The term “adeno-associated virus” or “AAV” as used herein refers to a member of the class of viruses associated with this name and belonging to the genus Dependoparvovirus, family Parvoviridae. Adeno-associated virus is a single-stranded DNA virus that grows only in cells in which certain functions are provided by a co-infecting helper virus. All AAV serotypes apparently exhibit very similar replication properties mediated by homologous rep genes; and all bear three related capsid proteins. At least 13 sequentially numbered naturally-occurring AAV serotypes are known in the art. Non-limiting exemplary serotypes useful in the methods disclosed herein include any of those 13 serotypes, e.g., AAV2, AAV8, AAV9, or variant serotypes, e.g., AAV-DJ and AAV PHP.B. The AAV particle comprises, consists essentially of, or consists of three major viral proteins: VP1, VP2 and VP3. In embodiments, the AAV refers to the serotype AAV1, AAV2, AAV3, AAV4, AAV5, AAV6, AAV7, AAV8, AAV9, AAV10, AAV11, AAV12, or AAV13. In embodiments, the AAV particle comprises an AAV capsid protein selected from the group consisting of AAVPHP.B, AAVrh74, AAV 110, AAV 204, AAV 214, AAV 214A, AAV 214e, AAV 214e8, AAV 214e9, AAV 214e10, AAV ITB102_45, and AAV 214AB. In embodiments, the AAV refers to the serotype AAV1, AAV2, AAV3, AAV3B, AAV4, AAV5, AAV6, AAV7, AAV8, AAV9, AAV10, AAV11, AAV12, or AAV13, AAVrh10, AAVhu37 or any one of the AAV serotypes isolated from human and nonhuman mammalians or variant thereof. In embodiments, the AAV particle comprises an AAV capsid protein selected from the group consisting of: AAV1, AAV2, AAV2G9, AAV3, AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/ri1.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAV-DJ, AAV-DJ8, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10, Japanese AAV 10 serotypes, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv 1-1, AAV Clvl-10, AAV CLv 1-2, AAV CLv-12, AAV CLv 1-3, AAV CLv-13, AAV CLv 1-4, AAV Clvl-7, AAV Clvl-8, AAV Clvl-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAVhu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, AAVF9/HSC9, AAV-PHP.B (PHP.B), AAV-PHP.A (PHP.A), G2B-26, G2B-13, TH1.1-32, TH1.1-35, AAVPHP.B2, AAVPHP.B3, AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.S/G2A12, AAVG2A15/G2A3, AAVG2B4, AAVG2B5 and variants thereof.

An “AAV vector” as used herein refers to a vector comprising one or more heterologous nucleic acid (HNA) sequences and one or more AAV inverted terminal repeat sequences (ITRs). Such AAV vectors can be replicated in a host cell that provides the functionality of rep and cap gene products, and allow the ITRs and the nucleic acid between the ITRs to be packaged into infectious viral particles. In embodiments, AAV vectors comprise a promoter, at least one nucleic acid that may encode at least one protein or RNA, and/or an enhancer and/or a terminator within the flanking ITRs that is packaged into the infectious AAV particle. The ITRs and the nucleic acid between the ITRs may be encapsulated into the AAV capsid, and this encapsidated nucleic acid may be referred to as the “AAV vector genome.” AAV vectors may contain elements in addition to the encapsidated portion, for example, antibiotic resistance genes or other elements known in the art included in the plasmid for manufacturing purposes but not packaged into the AAV particle.

As used herein, the term “viral capsid” or “capsid” refers to the proteinaceous shell or coat of a viral particle. Capsids function to encapsidate, protect, transport, and/or release into the host cell a viral genome. Capsids are generally comprised of oligomeric structural subunits of protein (“capsid proteins”). The viral capsid of AAV is composed of a mixture of three viral capsid proteins: VP1, VP2, and VP3.

An “AAV virion” or “AAV viral particle” or “AAV particle” refers to a viral particle composed of at least one AAV capsid protein and an encapsidated polynucleotide from an AAV vector referred to herein as the AAV vector genome.

A “subject” of diagnosis or treatment is an animal such as a mammal, or a human. A subject is not limited to a specific species and includes non-human animals subject to diagnosis or treatment and those subject to infections or animal models, including, without limitation, simian, murine, rat, canine, or leporid species, as well as other livestock, sport animals, or pets. In embodiments, the subject is a human.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease) progression, amelioration or palliation of the condition (including disease) states and remission (whether partial or total), whether detectable or undetectable.

As used herein the term “effective amount” intends to mean a quantity sufficient to achieve a desired effect. In the context of therapeutic or prophylactic applications, the effective amount may depend on the type and severity of the condition at issue and the characteristics of the individual subject, such as general health, age, sex, body weight, and tolerance to pharmaceutical compositions. In the context of gene therapy, in embodiments an effective amount is an amount sufficient to result in gaining partial or full function of a gene that is deficient in a subject. In other embodiments, the effective amount of an AAV viral particle is the amount sufficient to result in expression of a gene in a subject. The skilled artisan will be able to determine appropriate amounts depending on these and other factors.

In embodiments, the effective amount will depend on the size and nature of the application in question. It will also depend on the nature and sensitivity of the target subject and the methods in use. The skilled artisan will be able to determine the effective amount based on these and other considerations. The effective amount may comprise, consist essentially of, or consist of one or more administrations of a composition depending on the embodiment.

As used herein, the term “administering”, “administered”, or “administration” intends to mean delivery’ of a substance to a subject such as an animal or human. Administration can be affected in one dose, continuously or intermittently throughout the course of treatment. Methods of determining the most effective means and dosage of administration are known to those of skill in the art and wall vary with the composition used for therapy, the purpose of the therapy, as well as the age, health or gender of the subject being treated. Single or multiple administrations can be carried out with the dose level and pattern being selected by the treating physician or in the case of pets and other animals, treating veterinarian.

### EXAMPLES

Unless specified otherwise, the following general methods were followed in the examples described below.

The rAAV Production

AAV8 and AAV9 particles were produced by transient triple-transfection of HEK293T cells or suspension HEK293 cells with plasmids encoding the AAV Rep and Cap proteins, as well as the adenoviral helper genes, as well the recombinant genome containing the GBA1 construct. rAAV particles were purified using iodixanol based density gradient ultracentrifugation method. Subsequently, rAAV was then quantified by probe-based ddPCR (Biorad) assay and characterized by silver staining.

In Vitro Transfection and r-AAV Potency Assay

The day before transfection, HEK293T or the liver hepatocyte cell line HepG2 and Huh7 were plated in a 24-well plate at a cell density of 1.5E5 cells/well. Each well received 500 μl of complete cell culture medium. For transfection, a PEI-based transfection reagent was used. Specifically, 0.15 g of plasmids containing transgene sequence, as well as 0.15 g of plasmids containing Luciferase reporter gene, was co-transfected into each well. 48 h after transfection, 3001 of fresh complete cell culture medium was added to each well, and cells were incubated for additional 24 hours.

For dual-Luciferase reporter assay, cells were lysed by using cell lysis buffer (TransGen) and proceeded to Firefly Luciferase and Renilla Luciferase detection by using Luciferase detection system (TransGen). The 96-well plates containing cell lysate and detection reagent were read on Varioskan LUX reader (ThermoFisher). All Firefly Luciferase activity results were first normalized to corresponding Renilla Luciferase intensity. The results were then normalized to the control group.

For GCase activity assay, cell culture supernatants were proceeded to enzyme activity assay according to the method described below, cells were lysed by using cell lysis buffer (Promega) and proceeded to Luciferase detection by using Steady-Glo Luciferase detection system (Promega). The 96-well plates containing cell lysate and detection reagent were read on Varioskan LUX reader (ThermoFisher). All enzyme activity results were first normalized to corresponding Firefly Luciferase intensity. The results were then normalized to the control group.rAAV biopotency assay was performed by cell transduction using HEK293T, Huh7 or HepG2 cells. Cells were plated in 24-well plate at a cell density of 1.5E5 cells/well 24 hours before transduction. rAAV transduction was performed at defined multiplicity of infection (MOI) of 1E5 or 1E6. 48 h after infection, 3001 of fresh complete cell culture medium was added to each well, and cells were incubated for another 24 hours. 72 h after infection, cell culture supernatant was proceeded to enzyme activity test according to the method described below.

### Wild Type Mouse Study Design

AAV vector containing the GBA1 transgene were administered through tail vein injection of wild type (C57BL/6) male mice at age of 8-9 weeks. AAV dose was 1E12 vg/ml. To assess the kinetics and durability of transgene expression, serum GCase levels were measured at various time intervals (1, 2, or 4 weeks) post injection. Mice were followed up to 4 weeks post AAV treatment and sacrificed for biochemical and pathological analysis.

### Gaucher Mouse Study Design

AAV vector containing the GBA1 transgene were administered through tail vein injection of Gaucher (a combination of two different types of GBA1 mutations) mice at age of 7-12 weeks old. All mice were maintained under a special pathogen-free environment and in individually ventilated cages. All cages, cob bedding, and water were sterilized before use. The cages, cob bedding, food and water will be changed twice a week.

The AAV dose was ranged from 2E11 to 5E13 vg/ml. To assess the kinetics and durability of transgene expression, serum GCase levels and substrate accumulation levels were measured at various time intervals post injection. Mice were followed up to the end point of study and sacrificed for biochemical and pathological analysis.

### AAV/Cerezyme Preparation and Administration

Aliquots of rAAV were stored at −80° C. Before injection, the aliquot was thawed on ice and diluted with the AAV formulation buffer. The diluted AAV was kept on ice before injection and used within 2 hours.

Cerezyme was resuspended according to the manufacturer's instructions and aliquoted (401U/ml) and stored at −80° C. Before injection, the aliquot was thawed on ice and diluted, and gently but thoroughly mixed.

Serum and Tissue Collection Serum was separated from fresh blood without anticoagulation within 0.5 hour at 4° C. by centrifugation at 12,000 rpm for 15 mins. Serum was stored at −80° C. For the Cerezyme group, serum was collected 1.5 hours after injection.

Mice were anesthetized and euthanized. Tissues were collected from the mice after perfusion with saline and stored at −80° C. For the Cerezyme group, tissue samples were collected 1.5 hours after injection. Tissue samples were divided into 4 parts, with 3 parts frozen in individual tubes and stored at −80° C. for GCase activity assay, glucosylsphingosine analysis and mRNA analysis. The remaining part was fixed in 10% neutral buffered formalin solution (NBF, pH 7.4) for about 24-48 h at room temperature for histology analysis. Bone marrow were collected from femurs and tibias of both legs of the mice.

### Mice Serum and Tissue GBA1 Activity Assay

Serum samples were obtained from mouse blood and stored at −80° C. Tissues were lysed in tissue lysis buffer (Citrate-phosphate buffer, pH5.0, 0.25% Sodium taurocholic, 1% TX-100 with Proteinase inhibitor cocktail) using a homogenizer (Shanghai jingxin) at specific program (50 Hz, working 30s and cooling down 30s, 4 min totally). For the enzyme activity assay, 0-Glucocerebrosidase (acid P-glucosidase; GCase) activity was determined by fluorescence based assay. 4-Methylumbelliferyl P-D-glucopyranoside (4MU-Glc, Carbosynth) served as the substrate for enzyme GCase. On the day of the assay, serum was diluted 1:100 using enzyme assay buffer (Citrate-phosphate buffer, pH5.0, 0.25% Sodium taurocholic, 0.25% TX-100). Tissue lysate was diluted 1:40 using lysis buffer (Citrate-phosphate buffer, pH5.0, 0.25% Sodium taurocholic, 1% TX-100 with Proteinase inhibitor cocktail). All samples were assayed in Citrate-phosphate buffer, pH5.0, 0.25% Sodium taurocholic, 0.25% TX-100, 1 mM 4MU-Glc, for 1 hour at 37° C. The reaction was stopped by adding three-fold volume (150 ul) of stop solution (0.5 M Glycine, pH 10.8). Relative fluorescence levels (RFU) were evaluated with a Varioskan LUX reader (ThermoFisher) using excitation and emission wavelengths of 360 nm and 460 nm, respectively. Tissue lysate samples were also proceeded to protein concentration assay by BCA kit (Thermofisher). Fluorescence levels were then converted to nmol/h/ml (serum) or nmol/h/mg of total protein (liver, spleen, lung, bone marrow and brain) based on a 4-Methylumbelliferone (4-MU, Sigma-Aldrich) standard curve.

### Vector Genome Copy Number, Relative RNA Transcription Level

To determine the vector genomic copy number in tissue samples post-rAAV injection, DNA was isolated from frozen liver samples using DNeasy Blood and Tissue Kit (QIAGEN) following manufacturers' instructions. Following DNA isolation, probe-based qPCR (Roche) was performed to determine the vector genome copy number/reaction. Cell number/reaction is calculated according to the DNA amount quantification results. Vector genome copy number/cell was then calculated by the normalization of genome cope/reaction to cell number/reaction.

To determine the relative RNA transcription level in tissue samples post-rAAV injection, RNA was isolated from frozen liver samples using RNeasy Kit (QIAGEN) following manufacturers' instructions. Following RNA isolation, cDNA was synthesized by using Primescript RT master mix (TAKARA). 300-500 ng RNA was applied to each RT reaction. cDNA was then diluted and been applied to probe-based qPCR (Roche) test.

### Immunohistochemistry

Rabbit anti-mouse CD68 antibody (1:25 Abcam AB53444) was used to visualize mouse macrophage. The formalin-fixed mouse tissues were deparaffined with xylene and graded ethanol washes, followed by antigen retrieval using pepsin according to product use recommendations. Sections were counterstained with haematoxylin. Biotin labeled secondary antibody was used for detecting. The signals were visualized by using Streptavidin-TRP and Tyramide signal amplification kit according to recommendations.

### Storage Cell Count

Tissue sections were stained with hematoxylin and eosin (H&E). The stained tissues were scanned with Aperio AT2 (Leica, 40×). The tissue images were processed with Aperio ImageScope (V12.4.3.5008). All Gaucher cells on a whole tissue section for both liver and lung per mouse are counted manually. Gaucher cell counts from the whole section was normalized to the tissue slice area (square centimeter) for data graph.

### Glucosylsphingosine(Lyso_GLI) Analysis

Tissue homogenate was prepared by homogenizing with 9 volumes (w:v) of PBS buffer. Aliquots (10 pL) of tissue lysates or serum sample were subjected to LC/MS analysis. The quantitated tissue Lyso_GL1 were normalized by tissue weight, and substrate level in serum was normalized by serum volume. Values bellowed the lower quantitation limit of Lyso_GL 1 (LLOQ) of 10 ng/g for tissues and 1 ng/ml for serum or plasma will be labelled as BQL in the corresponding figures.

### Statistical Analysis

Data were presented as mean±standard error of the mean (mean±SEM). The GraphPad Prism software was used for statistical analysis of the differences among groups. The p value ≤0.05 was considered statically significant.

### Example 1: Constructs

To improve the therapeutic outcomes of gene therapy for Gaucher disease caused by metabolic disturbance, this disclosure outlines an integrated approach for designing and screening novel expression cassettes that efficiently and selectively express therapeutic GCase in the liver.

The first step involves cloning the nucleotide sequences of Firefly Luciferase gene and GBA1 gene into target vectors. To confine the Luciferase and GCase protein expression to the liver, the nucleotide sequences of Firefly Luciferase gene and GBA1 gene were driven by a series of chimeric hepatic specific regulatory elements (CHSREs) that were rationally designed with the combination of different promoters and various regulatory elements. These promoters described herein are HSRE001, HSRE002, HSRE003, HSRE004, HSRE005, HSRE015, HSRE016. The regulatory elements described herein are HSRE006, HSRE007, HSRE008, HSRE009, HSRE010, HSRE011, HSRE012, HSRE013 and HSRE014. Introns were also employed in some embodiments to further increase the GCase expression. The different promoters and various regulatory elements ID as well as their nucleotide sequences used in the present discourse are shown in Table 1. Codon-optimized GBA1 gene with K321N mutation is an Exemplary single nucleotide sequence of interest.

TABLE 1
Different regulatory elements used in the present discourse

| Regulatory | | | SEQ |
|---|---|---|---|
| Elements ID | Description | Nucleotide sequence | ID NO |
| CRE001 | / | cgttacataacttacggtaaatggcccgcctggctgaccgcccaacgacccccgcccat | 1 |
| | | tgacgtcaatagtaacgccaatagggactttccattgacgtcaatgggtggagtattta | |
| | | cggtaaactgcccacttggcagtacatcaagtgtatcatatgccaagtacgccccctat | |
| | | tgacgtcaatgacggtaaatggcccgcctggcattgtgcccagtacatgaccttatggg | |
| | | actttcctacttggcagtacatctacgtattagtcatcgctattaccatggtcgaggtg | |
| | | agccccacgttctgcttcactctccccatctcccccccctccccacccccaattttgta | |
| | | tttatttattttttaattattttgtgcagcgatgggggcggggggggggggggggcgcg | |
| | | cgccaggcggggggggcggggcgaggggcggggcggggcgaggcggagaggtgcggcgg | |
| | | cagccaatcagagcggcgcgctccgaaagtttccttttatggcgaggcggcggcggcgg | |
| | | cggccctataaaaagcgaagcgcgcggggggggagtcgctgcgcgctgccttcgccccg | |
| | | tgccccgctccgccgccgcctcgcgccgcccgccccggctctgactgaccgcgttactc | |
| | | ccacaggtgagcggggggacggcccttctcctccgggctgtaattagctgagcaagagg | |
| | | taagggtttaagggatggttggttggtggggtattaatgtttaattacctggagcacct | |
| | | gcctgaaatcactttttttcaggttgg | |
| HSRE001 | / | gtctgtctgcacatttogtagagcgagtgttccgatactctaatctccctaggcaaggt | 2 |
| | | tcatatttgtgtaggttacttattctccttttgttgactaagtcaataatcagaatcag | |
| | | caggtttggagtcagcttggcagggatcagcagcctgggttggaaggagggggtataaa | |
| | | agccccttcaccaggagaagccgtcacacagatccacaagctcct | |
| HSRE002 | / | gtctgtctgcacatttcgtagagcgagtgttccgatactctaatctccctaggcaaggt | 3 |
| | | tcatattgacttaggttacttattctccttttgttgactaagtcaataatcagaatcag | |
| | | caggtttggagtcagcttggcagggatcagcagcctgggttggaaggagggggtataaa | |
| | | agccccttcaccaggagaagccgtcacacagatccacaagctcct | |
| HSRE003 | / | ccaccccctccaccttggacacaggacgctgtggtttctgagccaggtacaatggggcg | 4 |
| | | actcagatcccagccagtggacttagcccctgtttgctcctccgataactggggtgacc | |
| | | ttggttaatattcaccagcagcctcccccgttgcccctctggatccactgcttaaatac | |
| | | ggacgaggacagggccctgtctcctcagcttcaggcaccaccactgacctgggacagtg | |
| | | aat | |
| HSRE004 | / | gatcttgctaccagtggaacagccactaaggattctgcagtgagagcagagggccagct | 5 |
| | | aagtggtactctcccagagactgtctgactcacgccaccccctccaccttggacacagg | |
| | | acgctgtggtttctgagccaggtacaatgactcctttcggtaagtgcagtggaagctgt | |
| | | acactgcccaggcaaagcgtccgggcagcgtaggcgggcgactcagatcccagccagtg | |
| | | gacttagcccctgtttgctcctccgataactggggtgaccttggttaatattcaccagc | |
| | | agcctcccccgttgcccctctggatccactgcttaaatacggacgaggacagggccctg | |
| | | tctcctcagcttcaggcaccaccactgacctgggacagtgaa | |
| HSRE005 | / | tgcatgtataatttctacagaacctattagaaaggatcacccagcctctgcttttgtac | 6 |
| | | aactttcccttaaaaaactgccaattccactgctgtttggcccaatagtgagaactttt | |
| | | tcctgctgcctcttggtgcttttgcctatggcccctattctgcctgctgaagacactct | |
| | | tgccagcatggacttaaacccctccagctctgacaatcctctttctcttttgttttaca | |
| | | tgaagggtctggcagccaaagcaatcactcaaagttcaaaccttatcattttttgcttt | |
| | | gttcctcttggccttggttttgtacatcagctttgaaaataccatcccagggttaatgc | |
| | | tggggttaatttataactaagagtgctctagttttgcaatacaggacatgctataaaaa | |
| | | tggaaagatgttgctttctgagagat | |
| HSRE006 | / | gggggaggctgctggtgaatattaaccaaggtcaccccagttatoggaggagcaaacag | 7 |
| | | gggctaagtccac | |
| HSRE007 | Three copies | gggggaggctgctggtgaatattaaccaaggtcaccccagttatoggaggagcaaacag | 8 |
| | of HSRE006 | gggctaagtccacgggggaggctgctggtgaatattaaccaaggtcaccccagttatcg | |
| | | gaggagcaaacaggggctaagtccacgggggaggctgctggtgaatattaaccaaggtc | |
| | | accccagttatoggaggagcaaacaggggctaagtccac | |
| HSRE008 | Six copies of | gggggaggctgctggtgaatattaaccaaggtcaccccagttatcggaggagcaaacag | 9 |
| | HSRE006 | gggctaagtccacgggggaggctgctggtgaatattaaccaaggtcaccccagttatcg | |
| | | gaggagcaaacaggggctaagtccacgggggaggctgctggtgaatattaaccaaggtc | |
| | | accccagttatcggaggagcaaacaggggctaagtccacgggggaggctgctggtgaat | |
| | | attaaccaaggtcaccccagttatcggaggagcaaacaggggctaagtccacgggggag | |
| | | gctgctggtgaatattaaccaaggtcaccccagttatcggaggagcaaacaggggctaa | |
| | | gtccacgggggaggctgctggtgaatattaaccaaggtcaccccagttatoggaggagc | |
| | | aaacaggggctaagtccac | |
| HSRE009 | / | gttaatttttaaaaagcaagtggcccttggcagcatctgtttgctctggttaataatct | 10 |
| | | caggagcacaaacattcccaggagaagaaatcaacatcctggacttatcctctgggcct | |
| HSRE010 | | gcactgggaggatgttgagtaagatggaaaactactgatgacccttgcagagacagagt | 11 |
| | | attaggacatgtttgaacaggggccgggcgatcagcaggtagctctagaggatcccc | |
| HSRE011 | Two copies of | gcactgggaggatgttgagtaagatggaaaactactgatgacccttgcagagacagagt | 12 |
| | HSRE010 | attaggacatgtttgaacaggggccgggcgatcagcaggtagctctagaggatccccgc | |
| | | actgggaggatgttgagtaagatggaaaactactgatgacccttgcagagacagagtat | |
| | | taggacatgtttgaacaggggccgggcgatcagcaggtagctctagaggatcccc | |
| HSRE012 | / | aggctcagaggcacacaggagtttctgggctcaccctgcccccttccaacccctcagtt | 13 |
| | | cccatcctccagcagctgtttgtgtgctgcctctgaagtccacactgaacaaacttcag | |
| | | cctactcatgtccctaaaatgggcaaacattgcaagcagcaaacagcaaacacacagcc | |
| | | ctccctgcctgctgaccttggagctggggcagaggtcagagacctctctgggcccatgc | |
| | | cacctccaacatccactcgaccccttggaatttcggtggagaggagcagaggttgtcct | |
| | | ggcgtggtttaggtagtgtgagaggg | |
| HSRE013 | Two copies of | aggctcagaggcacacaggagtttctgggctcaccctgcccccttccaacccctcagtt | 14 |
| | HSRE012 | cccatcctccagcagctgtttgtgtgctgcctctgaagtccacactgaacaaacttcag | |
| | | cctactcatgtccctaaaatgggcaaacattgcaagcagcaaacagcaaacacacagcc | |
| | | ctccctgcctgctgaccttggagctggggcagaggtcagagacctctctgggcccatgc | |
| | | cacctccaacatccactcgaccccttggaatttcggtggagaggagcagaggttgtcct | |
| | | ggcgtggtttaggtagtgtgagagggaggctcagaggcacacaggagtttctgggctca | |
| | | ccctgcccccttccaacccctcagttcccatcctccagcagctgtttgtgtgctgcctc | |
| | | tgaagtccacactgaacaaacttcagcctactcatgtccctaaaatgggcaaacattgc | |
| | | aagcagcaaacagcaaacacacagccctccctgcctgctgaccttggagctggggcaga | |
| | | ggtcagagacctctctgggcccatgccacctccaacatccactcgaccccttggaattt | |
| | | cggtggagaggagcagaggttgtcctggcgtggtttaggtagtgtgagaggg | |
| HSRE014 | / | ctgcaggctcagaggcacacaggagtttctgggctcaccctgcccccttccaacccctc | 15 |
| | | agttcccatcctccagcagctgtttgtgtgctgcctctgaagtccacactgaacaaact | |
| | | tcagcctactcatgtccctaaaatgggcaaacattgcaagcagcaaacagcaaacacac | |
| | | agccctccctgcctgctgaccttggagctggggcagaggtcagagacctctctgggccc | |
| | | atgccacctccaacatccactcgaccccttggaatttoggtggagaggagcagaggttg | |
| | | tcctggcgtggtttaggtagtgtgagagggtccgggttcaaaaccacttgctgggtggg | |
| | | gagtcgtcagtaagtggctatgccccgaccccgaagcctgtttccccatctgtacaatg | |
| | | gaaatgataaagacgcccatctgatagggtttttgtggcaaataaacatttggtttttt | |
| | | tgttttgttttgttttgttttttgagatggaggtttgctctgtcgcccaggctggagtg | |
| | | cagtgacacaatctcatctcaccacaaccttcccctgcctcagcctcccaagtagctgg | |
| | | gattacaagcatgtgccaccacacctggctaattttctatttttagtagagacgggttt | |
| | | ctccatgttggtcagcctcagcctcccaagtaactgggattacaggcctgtgccaccac | |
| | | acccggctaattttttctatttttgacagggacggggtttcaccatgttggtcaggctg | |
| | | gtctaga | |
| HSRE015 | / | caggttaatttttaaaaagcagtcaaaagtccaagtggcccttggcagcatttactctc | 16 |
| | | tctgtttgctctggttaataatctcaggagcacaaacattccagatccaggttaatttt | |
| | | taaaaagcagtcaaaagtccaagtggcccttggcagcatttactctctctgtttgctct | |
| | | ggttaataatctcaggagcacaaacattccagatccggcgcgccagggctggaagctac | |
| | | ctttgacatcatttcctctgcgaatgcatgtataatttctacagaacctattagaaagg | |
| | | atcacccagcctctgcttttgtacaactttcccttaaaaaactgccaattccactgctg | |
| | | tttggcccaatagtgagaactttttcctgctgcctcttggtgcttttgcctatggcccc | |
| | | tattctgcctgctgaagacactcttgccagcatggacttaaacccctccagctctgaca | |
| | | atcctctttctcttttgttttacatgaagggtctggcagccaaagcaatcactcaaagt | |
| | | tcaaaccttatcattttttgctttgttcctcttggccttggttttgtacatcagctttg | |
| | | aaaataccatcccagggttaatgctggggttaatttataactaagagtgctctagtttt | |
| | | gcaatacaggacatgctataaaaatggaaagat | |
| HSRE016 | / | aggctcagaggcacacaggagtttctgggctcaccctgcccccttccaacccctcagtt | 17 |
| | | cccatcctccagcagctgtttgtgtgctgcctctgaagtccacactgaacaaacttcag | |
| | | cctactcatgtccctaaaatgggcaaacattgcaagcagcaaacagcaaacacacagcc | |
| | | ctccctgcctgctgaccttggagctggggcagaggtcagagacctctctgggcccatgc | |
| | | cacctccaacatccactcgaccccttggaatttoggtggagaggagcagaggttgtcct | |
| | | ggcgtggtttaggtagtgtgagaggggtacccggggatottgctaccagtggaacagcc | |
| | | actaaggattctgcagtgagagcagagggccagctaagtggtactctcccagagactgt | |
| | | ctgactcacgccaccccctccaccttggacacaggacgctgtggtttctgagccaggta | |
| | | caatgactcctttcggtaagtgcagtggaagctgtacactgcccaggcaaagcgtccgg | |
| | | gcagcgtaggcgggcgactcagatcccagccagtggacttagcccctgtttgctcctcc | |
| | | gataactggggtgaccttggttaatattcaccagcagcctcccccgttgcccctctgga | |
| | | tccactgcttaaatacggacgaggacagggccctgtctcctcagcttcaggcaccacca | |
| | | ctgacctgggacagtgaa | |

TABLE 2
Nucleotide sequence of Luciferase and human GBA1

| | | | SEQ ID | SEQ ID |
|---|---|---|---|---|
| | | | NO of | NO of |
| | | | target gene | target |
| | | Nucleotide sequence of Luciferase or GBAI with | without | gene with |
| Constructs | | signal peptide (the underline is shown | signal | signal |
| ID | Gene Name | as the nucleotide sequence of signal peptide) | peptide | peptide |
| PG001 | wtGBA | atggagttttcaagtccttccagagaggaatgtcccaagcctttga | 18 | 19 |
| | (Wild-type | gtagggtaagcatcatggctggcagcctcacaggattgcttctact | | |
| | human | tcaggcagtgtcgtgggcatcaggtgcccgcccctgcatccctaaa | | |
| | GBA1 | agcttcggctacagctcggtggtgtgtgtctgcaatgccacatact | | |
| | nucleotide | gtgactcctttgaccccccgacctttcctgcccttggtaccttcag | | |
| | sequence, | ccgctatgagagtacacgcagtgggcgacggatggagctgagtatg | | |
| | with signal | gggcccatccaggctaatcacacgggcacaggcctgctactgaccc | | |
| | peptide | tgcagccagaacagaagttccagaaagtgaagggatttggaggggc | | |
| | portion, | catgacagatgctgctgctctcaacatccttgccctgtcaccccct | | |
| | from | gcccaaaatttgctacttaaatcgtacttctctgaagaaggaatcg | | |
| | Genebank | gatataacatcatccgggtacccatggccagctgtgacttctccat | | |
| | NM_0001577.3) | ccgcacctacacctatgcagacacccctgatgatttccagttgcac | | |
| | | aacttcagcctcccagaggaagataccaagctcaagatacccctga | | |
| | | ttcaccgagccctgcagttggcccagcgtcccgtttcactccttgc | | |
| | | cagcccctggacatcacccacttggctcaagaccaatggagcggtg | | |
| | | aatgggaaggggtcactcaagggacagcccggagacatctaccacc | | |
| | | agacctgggccagatactttgtgaagttcctggatgcctatgctga | | |
| | | gcacaagttacagttctgggcagtgacagctgaaaatgagccttct | | |
| | | gctgggctgttgagtggataccccttccagtgcctgggcttcaccc | | |
| | | ctgaacatcagcgagacttcattgcccgtgacctaggtcctaccct | | |
| | | cgccaacagtactcaccacaatgtccgcctactcatgctggatgac | | |
| | | caacgcttgctgctgccccactgggcaaaggtggtactgacagacc | | |
| | | cagaagcagctaaatatgttcatggcattgctgtacattggtacct | | |
| | | ggactttctggctccagccaaagccaccctaggggagacacaccgc | | |
| | | ctgttccccaacaccatgctctttgcctcagaggcctgtgtgggct | | |
| | | ccaagttctgggagcagagtgtgcggctaggctcctgggatcgagg | | |
| | | gatgcagtacagccacagcatcatcacgaacctcctgtaccatgtg | | |
| | | gtcggctggaccgactggaaccttgccctgaaccccgaaggaggac | | |
| | | ccaattgggtgcgtaactttgtcgacagtcccatcattgtagacat | | |
| | | caccaaggacacgttttacaaacagcccatgttctaccaccttggc | | |
| | | cacttcagcaagttcattcctgagggctcccagagagtggggctgg | | |
| | | ttgccagtcagaagaacgacctggacgcagtggcactgatgcatcc | | |
| | | cgatggctctgctgttgtggtcgtgctaaaccgctcctctaaggat | | |
| | | gtgcctottaccatcaaggatcctgctgtgggcttcctggagacaa | | |
| | | tctcacctggctactccattcacacctacctgtggogtcgccagtg | | |
| | | a | | |
| PG011 | mGBA- | atggagttctcaagtccttctcgcgaggaatgtcccaaaccactga | 20 | 21 |
| | C110 | gccgcgtgtcaattatggctggatctctgaccggactgctgctgct | | |
| | | gcaggccgtgagttgggctagtggggcccggccctgcatcccaaag | | |
| | | agctttggctacagctctgtggtgtgcgtgtgcaacgccacctact | | |
| | | gcgacagtttcgaccctcccaccttcccagccctgggcacattctc | | |
| | | tcggtacgagtccactcgctcaggccggagaatggagctgtctatg | | |
| | | ggcccaatccaggctaatcataccggaacaggcctgctgctgacac | | |
| | | tgcagccagagcagaagtttcagaaggtgaaaggcttcgggggcgc | | |
| | | catgaccgatgctgctgccctgaatatcctggccctgagtccccca | | |
| | | gctcagaatctgctgctgaagtcatactttagtgaggaaggcattg | | |
| | | gatacaatattatccgggtgcctatggcctcctgtgacttctcaat | | |
| | | tcgcacctatacttacgctgatacccccgatgatttccagctgcac | | |
| | | aacttcagtctgcccgaggaagataccaaactgaaaatcccactga | | |
| | | tccatogggctctgcagctggctcagcgcccagtgagcctgctggc | | |
| | | ctctccttggacctcaccaacatggctgaagactaacggogccgtg | | |
| | | aacgggaaaggcagtctgaaagggcagccaggcgacatctatcacc | | |
| | | agacttgggcccggtacttcgtgaaatttctggatgcctacgctga | | |
| | | gcacaaactgcagttttgggccgtgaccgccgaaaatgagccatca | | |
| | | gccggcctgctgtctggctacccatttcagtgtctggggtttacac | | |
| | | cagagcatcagagggacttcatcgcccgggacctgggaccaaccct | | |
| | | ggccaacagcacccaccataacgtgaggctgctgatgctggacgat | | |
| | | cagcggctgctgctgcctcactgggctaaggtggtgctgaccgatc | | |
| | | cagaggctgccaagtacgtgcatggaatcgccgtgcactggtacct | | |
| | | ggatttcctggcccctgctaacgccaccctgggagagacccatogg | | |
| | | ctgttccccaacaccatgctgtttgcctctgaagcttgcgtggggt | | |
| | | caaagttttgggagcagtcagtgaggctgggcagttgggatcgggg | | |
| | | gatgcagtactcccacagtattatcaccaatctgctgtaccatgtg | | |
| | | gtgggctggaccgattggaatctggccctgaatccagaggggggcc | | |
| | | ctaattgggtgcggaacttogtggactccccaatcattgtggatat | | |
| | | tacaaaggacaccttctacaaacagcccatgttctaccacctgggc | | |
| | | cactttagtaagttcatccctgaggggtcacagcgggtgggcctgg | | |
| | | tggcctoccagaaaaacgacctggatgctgtggctctgatgcaccc | | |
| | | tgacggatcagccgtggtggtggtgctgaatcgctctagtaaggac | | |
| | | gtgccactgaccatcaaggatccagctgtgggcttcctggagacaa | | |
| | | tcagtcccgggtactctatccacacctatctgtggcgccggcagtg | | |
| | | a | | |
| | Firefly | atggaagacgccaaaaacataaagaaaggcccggcgccattctatc | 56 | / |
| | Luciferase | cgctggaagatggaaccgctggagagcaactgcataaggctatgaa | | |
| | | gagatacgccctggttcctggaacaattgcttttacagatgcacat | | |
| | | atogaggtggacatcacttacgctgagtacttcgaaatgtccgttc | | |
| | | ggttggcagaagctatgaaacgatatgggctgaatacaaatcacag | | |
| | | aatcgtcgtatgcagtgaaaactctcttcaattctttatgccggtg | | |
| | | ttgggcgcgttatttatcggagttgcagttgcgcccgcgaacgaca | | |
| | | tttataatgaacgtgaattgctcaacagtatgggcatttogcagcc | | |
| | | taccgtggtgttcgtttccaaaaaggggttgcaaaaaattttgaac | | |
| | | gtgcaaaaaaagctcccaatcatccaaaaaattattatcatggatt | | |
| | | ctaaaacggattaccagggatttcagtcgatgtacacgttcgtcac | | |
| | | atctcatctacctcccggttttaatgaatacgattttgtgccagag | | |
| | | tccttcgatagggacaagacaattgcactgatcatgaactcctctg | | |
| | | gatctactggtctgcctaaaggtgtcgctctgcctcatagaactgc | | |
| | | ctgcgtgagattctcgcatgccagagatcctatttttggcaatcaa | | |
| | | atcattccggatactgcgattttaagtgttgttccattccatcacg | | |
| | | gttttggaatgtttactacactoggatatttgatatgtggatttcg | | |
| | | agtcgtcttaatgtatagatttgaagaagagctgtttctgaggagc | | |
| | | cttcaggattacaagattcaaagtgcgctgctggtgccaaccctat | | |
| | | tctccttcttcgccaaaagcactctgattgacaaatacgatttatc | | |
| | | taatttacacgaaattgcttctggtggcgctcccctctctaaggaa | | |
| | | gtcggggaagcggttgccaagaggttccatctgccaggtatcaggc | | |
| | | aaggatatgggctcactgagactacatcagctattctgattacacc | | |
| | | cgagggggatgataaaccgggcgcggtcggtaaagttgttccattt | | |
| | | tttgaagcgaaggttgtggatctggataccgggaaaacgctgggcg | | |
| | | ttaatcaaagaggcgaactgtgtgtgagaggtcctatgattatgtc | | |
| | | cggttatgtaaacaatccggaagcgaccaacgccttgattgacaag | | |
| | | gatggatggctacattctggagacatagcttactgggacgaagacg | | |
| | | aacacttcttcatcgttgaccgcctgaagtctctgattaagtacaa | | |
| | | aggctatcaggtggctcccgctgaattggaatccatcttgctccaa | | |
| | | caccccaacatcttcgacgcaggtgtgcaggtcttcccgacgatga | | |
| | | cgccggtgaacttcccgccgccgttgttgttttggagcacggaaag | | |
| | | acgatgacggaaaaagagatcgtggattacgtcgccagtcaagtaa | | |
| | | caaccgcgaaaaagttgcgcggaggagttgtgtttgtggacgaagt | | |
| | | accgaaaggtcttaccggaaaactcgacgcaagaaaaatcagagag | | |
| | | atcctcataaaggccaagaagggcggaaagatcgccgtgtaa | | |

Polypeptide sequence of codon-optimized human
GBA1 without signal peptide portion but K321N
mutant
(SEQ ID NO: 22)
ARPCIPKSFGYSSVVCVCNATYCDSFDPPTFPALGTFSRYESTRSGRRM
ELSMGPIQANHTGTGLLLTLQPEQKFQKVKGFGGAMTDAAALNILALSP
PAQNLLLKSYFSEEGIGYNIIRVPMASCDFSIRTYTYADTPDDFQLHNF
SLPEEDTKLKIPLIHRALQLAQRPVSLLASPWTSPTWLKTNGAVNGKGS
LKGQPGDIYHQTWARYFVKFLDAYAEHKLQFWAVTAENEPSAGLLSGYP
FQCLGFTPEHQRDFIARDLGPTLANSTHHNVRLLMLDDQRLLLPHWAKV
VLTDPEAAKYVHGIAVHWYLDFLAPANATLGETHRLFPNTMLFASEACV
GSKFWEQSVRLGSWDRGMQYSHSIITNLLYHVVGWTDWNLALNPEGGPN
WVRNFVDSPIIVDITKDTFYKQPMFYHLGHFSKFIPEGSQRVGLVASQK
NDLDAVALMHPDGSAVVVVLNRSSKDVPLTIKDPAVGFLETISPGYSIH
TYLWRRQ*
Polypeptide sequence of codon-optimized human
GBA1 with signal peptide portion and K321N
mutant
(SEQ ID NO: 23)
MEFSSPSREECPKPLSRVSIMAGSLTGLLLLQAVSWASGARPCIPKSFG
YSSVVCVCNATYCDSFDPPTFPALGTFSRYESTRSGRRMELSMGPIQAN
HTGTGLLLTLQPEQKFQKVKGFGGAMTDAAALNILALSPPAQNLLLKSY
FSEEGIGYNIIRVPMASCDFSIRTYTYADTPDDFQLHNFSLPEEDTKLK
IPLIHRALQLAQRPVSLLASPWTSPTWLKTNGAVNGKGSLKGQPGDIYH
QTWARYFVKFLDAYAEHKLQFWAVTAENEPSAGLLSGYPFQCLGFTPEH
QRDFIARDLGPTLANSTHHNVRLLMLDDQRLLLPHWAKVVLTDPEAAKY
VHGIAVHWYLDFLAPANATLGETHRLFPNTMLFASEACVGSKFWEQSVR
LGSWDRGMQYSHSIITNLLYHVVGWTDWNLALNPEGGPNWVRNFVDSPI
IVDITKDTFYKQPMFYHLGHFSKFIPEGSQRVGLVASQKNDLDAVALMH
PDGSAVVVVLNRSSKDVPLTIKDPAVGFLETISPGYSIHTYLWRRQ*

### Example 2

### In Vitro Screening of Chimeric Hepatic Specific Promoters

To confine the expression of the Luciferase or GCase in the liver, a series of chimeric hepatic specific regulatory elements (CHSREs) comprising core promoter and one, two, three or more regulatory elements were well designed and underwent in vitro or in vivo screening by comparing the Luciferase or GCase expression efficiency in HepG2 cells. Firefly Luciferase or mGBA-C110 (a codon-optimized GBA1 version with K321N mutation) were expressed under the control of various CHSREs.

Chimeric hepatic regulatory element (CHSRE) comprising of one, two, three or multiple copy of enhancers selected from HSRE010, HSRE014, HSRE012, HSRE006, HSRE009 are then constructed with the combination of 4 promoters of HSRE002, HSRE004, HSRE003 or HSRE005 to drive the expression of Luciferase gene or GBA1 gene as the constructs of PG130-PG165 and PG023, PG024, PG025, PG026, PG027, PG028, PG029, PG030, PG033, PG034, PG035, PG036, PG037, PG038, PG040, PG041, PG042, PG043, PG044, PG045, PG047, PG048, PG049, PG050, PG051, PG052 for further examination of Luciferase or GCase expression in HepG2 cells. And These constructs information is shown in the Table 3.

TABLE 3
Constructs information

| | Chimeric | Information of | | | | SEQ ID NO |
|---|---|---|---|---|---|---|
| Constructs | Regulatory | Chimeric HSRE promoter (5′-3′) | | | | of Target |
| ID | Element ID | Enhancer3 | Enhancer2 | Enhancer 1 | Promoter | Gene |
| PG022 | CHSRE001 | / | / | / | HSRE002 | 21 |
| PG023 | CHSRE002 | / | HSRE007 | HSRE014 | HSRE002 | 21 |
| PG024 | CHSRE003 | / | HSRE009 | HSRE014 | HSRE002 | 21 |
| PG025 | CHSRE004 | / | HSRE010 | HSRE012 | HSRE002 | 21 |
| PG026 | CHSRE005 | / | HSRE007 | HSRE012 | HSRE002 | 21 |
| PG027 | CHSRE006 | / | HSRE009 | HSRE012 | HSRE002 | 21 |
| PG028 | CHSRE007 | / | HSRE011 | HSRE012 | HSRE002 | 21 |
| PG029 | CHSRE008 | / | HSRE008 | HSRE012 | HSRE002 | 21 |
| PG030 | CHSRE009 | HSRE010 | HSRE007 | HSRE012 | HSRE002 | 21 |
| PG032 | CHSRE011 | / | / | / | HSRE005 | 21 |
| PG033 | CHSRE012 | / | HSRE010 | HSRE014 | HSRE005 | 21 |
| PG034 | CHSRE013 | / | HSRE007 | HSRE014 | HSRE005 | 21 |
| PG035 | CHSRE014 | / | HSRE009 | HSRE014 | HSRE005 | 21 |
| PG036 | CHSRE015 | / | HSRE010 | HSRE012 | HSRE005 | 21 |
| PG037 | CHSRE016 | / | HSRE007 | HSRE012 | HSRE005 | 21 |
| PG038 | CHSRE017 | / | HSRE009 | HSRE012 | HSRE005 | 21 |
| PG039 | CHSRE018 | / | / | / | HSRE004 | 21 |
| PG040 | CHSRE019 | / | HSRE010 | HSRE014 | HSRE004 | 21 |
| PG041 | CHSRE020 | / | HSRE007 | HSRE014 | HSRE004 | 21 |
| PG042 | CHSRE021 | / | HSRE009 | HSRE014 | HSRE004 | 21 |
| PG043 | CHSRE022 | / | HSRE010 | HSRE012 | HSRE004 | 21 |
| PG044 | CHSRE023 | / | HSRE007 | HSRE012 | HSRE004 | 21 |
| PG045 | CHSRE024 | / | HSRE009 | HSRE012 | HSRE004 | 21 |
| PG046 | CHSRE025 | / | / | / | HSRE003 | 21 |
| PG047 | CHSRE026 | / | HSRE010 | HSRE014 | HSRE003 | 21 |
| PG048 | CHSRE027 | / | HSRE007 | HSRE014 | HSRE003 | 21 |
| PG049 | CHSRE028 | / | HSRE009 | HSRE014 | HSRE003 | 21 |
| PG050 | CHSRE029 | / | HSRE010 | HSRE012 | HSRE003 | 21 |
| PG051 | CHSRE030 | / | HSRE007 | HSRE012 | HSRE003 | 21 |
| PG052 | CHSRE031 | / | HSRE009 | HSRE012 | HSRE003 | 21 |
| PG053 | CHSRE032 | / | HSRE010 | HSRE012 | HSRE002 | 21 |
| | | | | located in | | |
| | | | | downstream | | |
| | | | | of | | |
| | | | | promoter | | |
| PG054 | CHSRE033 | / | HSRE007 | HSRE012 | HSRE002 | 21 |
| | | | | located in | | |
| | | | | downstream | | |
| | | | | of | | |
| | | | | promoter | | |
| PG055 | CHSRE034 | / | HSRE010 | HSRE013 | HSRE002 | 21 |
| PG056 | CHSRE035 | / | HSRE007 | HSRE013 | HSRE002 | 21 |
| PG057 | CHSRE036 | / | HSRE009 | HSRE013 | HSRE002 | 21 |
| PG130 | CHSRE001 | / | / | / | HSRE002 | 56 |
| PG131 | CHSRE004 | / | HSRE010 | HSRE012 | HSRE002 | 56 |
| PG132 | CHSRE005 | / | HSRE007 | HSRE012 | HSRE002 | 56 |
| PG133 | CHSRE032 | | HSRE010 | HSRE012 | HSRE002 | 56 |
| | | | | located in | | |
| | | | | downstream | | |
| | | | | of | | |
| | | | | promoter | | |
| PG134 | CHSRE033 | / | HSRE007 | HSRE012 | HSRE002 | 56 |
| | | | | located in | | |
| | | | | downstream | | |
| | | | | of | | |
| | | | | promoter | | |
| PG135 | CHSRE006 | / | HSRE009 | HSRE012 | HSRE002 | 56 |
| PG136 | CHSRE034 | / | HSRE010 | HSRE013 | HSRE002 | 56 |
| PG137 | CHSRE035 | / | HSRE007 | HSRE013 | HSRE002 | 56 |
| PG138 | CHSRE036 | / | HSRE009 | HSRE013 | HSRE002 | 56 |
| PG139 | CHSRE002 | / | HSRE007 | HSRE014 | HSRE002 | 56 |
| PG140 | CHSRE003 | / | HSRE009 | HSRE014 | HSRE002 | 56 |
| PG141 | CHSRE007 | / | HSRE011 | HSRE012 | HSRE002 | 56 |
| PG142 | CHSRE008 | / | HSRE008 | HSRE012 | HSRE002 | 56 |
| PG143 | CHSRE009 | HSRE010 | HSRE007 | HSRE012 | HSRE002 | 56 |
| PG145 | CHSRE011 | / | / | / | HSRE005 | 56 |
| PG146 | CHSRE012 | / | HSRE010 | HSRE014 | HSRE005 | 56 |
| PG147 | CHSRE013 | / | HSRE007 | HSRE014 | HSRE005 | 56 |
| PG148 | CHSRE014 | / | HSRE009 | HSRE014 | HSRE005 | 56 |
| PG149 | CHSRE015 | / | HSRE010 | HSRE012 | HSRE005 | 56 |
| PG150 | CHSRE016 | / | HSRE007 | HSRE012 | HSRE005 | 56 |
| PG151 | CHSRE017 | / | HSRE009 | HSRE012 | HSRE005 | 56 |
| PG152 | CHSRE018 | / | / | / | HSRE004 | 56 |
| PG153 | CHSRE019 | / | HSRE010 | HSRE014 | HSRE004 | 56 |
| PG154 | CHSRE020 | / | HSRE007 | HSRE014 | HSRE004 | 56 |
| PG155 | CHSRE021 | / | HSRE009 | HSRE014 | HSRE004 | 56 |
| PG156 | CHSRE022 | / | HSRE010 | HSRE012 | HSRE004 | 56 |
| PG157 | CHSRE023 | / | HSRE007 | HSRE012 | HSRE004 | 56 |
| PG158 | CHSRE024 | / | HSRE009 | HSRE012 | HSRE004 | 56 |
| PG159 | CHSRE025 | / | / | / | HSRE003 | 56 |
| PG160 | CHSRE026 | / | HSRE010 | HSRE014 | HSRE003 | 56 |
| PG161 | CHSRE027 | / | HSRE007 | HSRE014 | HSRE003 | 56 |
| PG162 | CHSRE028 | / | HSRE009 | HSRE014 | HSRE003 | 56 |
| PG163 | CHSRE029 | / | HSRE010 | HSRE012 | HSRE003 | 56 |
| PG164 | CHSRE030 | / | HSRE007 | HSRE012 | HSRE003 | 56 |
| PG165 | CHSRE031 | / | HSRE009 | HSRE012 | HSRE003 | 56 |

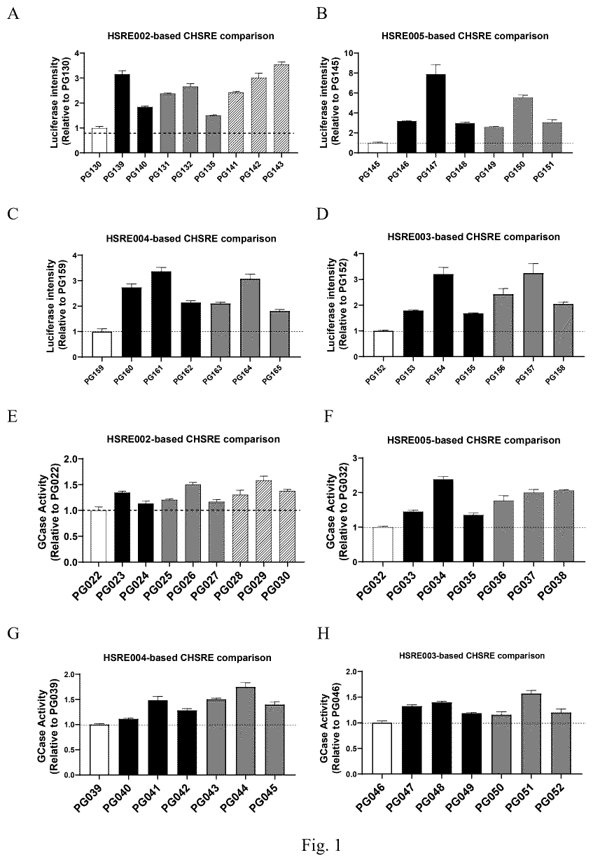

All of the Luciferase constructs with CHSREs showed higher Luciferase activity in comparison to the corresponding promoter-only versions (FIG. 1 A, B, C, D). When the Luciferase gene was replaced with the GBA1 gene, all of the GBA1 constructs except PG024-CHSRE003, PG027-CHSRE006 and PG050-CHSRE029 showed significantly higher GCase activity in comparison to the corresponding promoter-only versions (FIG. 1 E, F, G, H).

To determine if there is a further additive effect of these enhancers on the expression efficiency of Luciferase and GBA1 different copy number or further tandem combination of the enhancers were constructed into CHSREs. The results showed that further increasing copy number of HSRE010 (PG141-CHSRE007 vs. PG131-CHSRE004) or HSRE007 (PG142-CHSRE008 vs. PG132-CHSRE005) didn't affect the expression of Luciferase (FIG. 1A). Similarly, further increasing copy number of HSRE010 (PG028-CHSRE007 vs. PG025-CHSRE004) and HSRE007 (PG029-CHSRE008 vs. PG026-CHSRE005) have no effect on the expression of GCase (FIG. 1E). Further tandem adding HSRE010 to CHSRE005 as CHSRE009 in PG143 significantly augmented Luciferase expression compared to PG132-CHSRE005 (FIG. 1A), however, no effect on the GCase expression with CHSRE009 in PG030 was observed compared to the corresponding control of PG026-CHSRE005 (FIG. 1E).

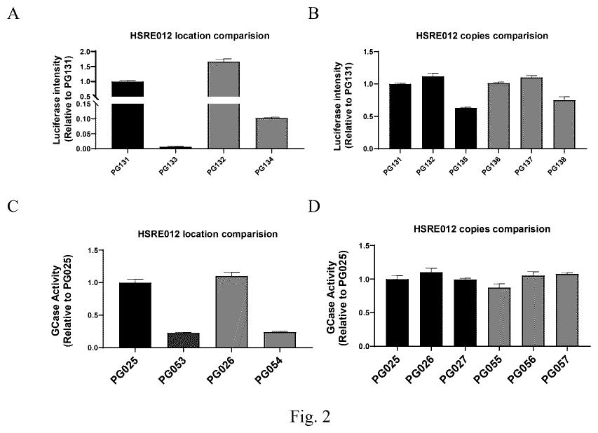

The effect of HSRE012 location relative to the HSRE002 promoter on Luciferase or GBA1 expression was tested and the results showed that the Luciferase or GCase expression was significantly decreased when the HSRE012 was moved from upstream (PG131-CHSRE004-luci and PG132-CHSRE005-luci, PG025-CHSRE004- and PG026-CHSRE005-) to downstream (PG133-CHSRE032-luci and PG134-CHSRE033-luci, PG053-CHSRE032- and PG054-CHSRE033-) of the promoter of HSRE002 (FIG. 2A, C). Increasing HSRE012 copy numbers in the CHSREs as PG136-CHSRE034-luci, PG137-CHSRE035-luci, PG138-CHSRE036-luci, PG055-CHSRE034-GBA1, PG056-CHSRE035-GBA1 and PG057-CHSRE036-GBA1 didn't further increase the expression of Luciferase or GCase compared to those corresponding constructs with single HSRE012 copy (PG131-CHSRE004-luci, PG132-CHSRE005-luci, PG135-CHSRE006-luci, PG025-CHSRE004-GBA1, PG026-CHSRE005-GBA1 and PG027-CHSRE006-GBA1) (FIG. 2B, D).

### Example 3

### In Vitro Screening for Introns

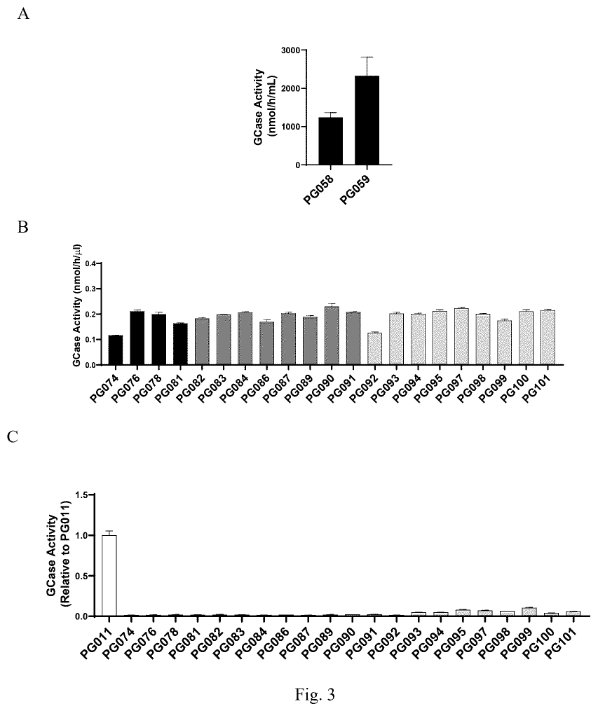

To further enhance the GCase expression, introns selected from human GBA1 derived endogenous introns of Int001 (inserted between 27-28 bp of SEQ ID NO:21), Int002 (inserted between 115-116 bp of SEQ ID NO:21), Int003 (inserted between 307-308 bp of SEQ ID NO:21), Int004 (inserted between 454-455 bp of SEQ ID NO: 21), Int005 (inserted between 588-589 bp of SEQ ID NO:21), Int006 (inserted between 761-762 bp of SEQ ID NO:21), Int007 (inserted between 999-1000 bp of SEQ ID NO:21), Int008 (inserted between 1224-1225 bp of SEQ ID NO:21), Int009 (inserted between 1388-1389 bp of SEQ ID NO:21) and Int0010 (inserted between 1505-1506 bp of SEQ ID NO:21) were cloned with mGBA-C110 under the promoter of HSRE002 as the constructs of PG059, PG060, PG061, PG062, PG063, PG064, PG065, PG066, PG067 and PG068, the construct without any intron is PG058. Endogenous introns selected from the group of Int002, Int003, Int005 and exogenous intron selected from Int011 and Int020 were cloned with mGBA-C110 under the promoters of HSRE002 as the constructs of PG069-PG073. The above constructs along with the PG058 and PG059 were transfected into Huh7 cells for the examination of GCase activity in cell culture supernatant. The PG059 constructs with Int001 inserted were proven to have higher GCase expression than the control without intron. (FIG. 3C).

Meanwhile, exogenous intron selected from group of Int012, Int013, Int014, Int015, Int016, Int017, Int018, Int019 and Int011 were cloned with mGBA-C110 under the hepatic specific promoters of HSRE001, HSRE015 and HSRE016 respectively as the constructs of PG074-101. These constructs with exogenous intron inserted upstream of GBA1 coding sequence were transfected in HepG2 cells and HEK293T cells for the examination of GCase activity in cell culture supernatant. The results showed that all constructs expressed GCase efficiently in HepG2 cells but very low in HEK293T cells (FIGS. 3A and 3B).

The results described above proved exogenous intron and GBA1 endogenous intron, when constructed with hepatic specific promoters, showed high expression of GCase in HepG2 cell, but all of which showed very weak GCase activity in HEK293T. The results described above indicated that these expression cassettes constructed with hepatic specific promoter and endogenous or exogenous intron described herein transcript GBA1 specifically in liver tissue.

TABLE 4
The sequences of Intron

| Intron | | SEQ |
|---|---|---|
| ID | Nucleotide sequence | ID |
| Int001 | gtaagagagagagctcccaatcagcattgtcacagtgcttctggaatcctggcactggaatttaatgaatgacagactct | 24 |
| | ctttgaatccagggccatcatggctctttgagcaaggcacagatggagggaggggtcgaagttgaaatgggtgggaagag | |
| | tggtggggagcatcctgatttggggtgggcagagagttgtcatcagaagggttgcagggagagctgcacccaggtttctg | |
| | tgggccttgtcctaatgaatgtgggagaccgggccatgggcacccaaaggcagctaagccctgcccaggagagtagttga | |
| | ggggtggagaggggcttgcttttcagtcattcctcattctgtcctcag | |
| Int002 | gtgagtgagtcaaggcagtggggaggtagcacagagcctcccttctgcctcatagtcctttggtagccttccagtaagct | 25 |
| | ggtggtagacttttagtaggtgctcaataaatccttttgagtgactgagaccaactttggggtgaggattttgttttttt | |
| | tcttttgaaacagagtcttactctgttgcctgggctggagtgcagtggtgcaattttggctcattccaacctctgcctcc | |
| | cagattcaagcgattctcttgcttcagcttcccaggtagctgggattacaggcggccaccactacgcccagctaattttt | |
| | gtatttttagtagagacggggtttcaccatgctggcaaggcaggtctcaaactcctcacctcaggtgatccgcccacctc | |
| | ggcctcctaaagtgctaggattacaggtgtgagcccctgcgcccggccaaggggtgaggaattttgaaaccgtgttcagt | |
| | ctctcctagcagatgtgtccattctccatgtcttcatcagacctcactctgcttgtactccctccctcccag | |
| Int003 | gtaaccattacacccctcaccccctgggccaggctgggtcctcctagaggtaaatggtgtcagtgatcaccatggagttt | 26 |
| | cccgctgggtactgatacccttattccctgtggatgtcctcag | |
| Int004 | gtgaggaggaaggggacaagatgacatagagccattgaaacttttcgtttttcttttctttttttaaaatttttttgagg | 27 |
| | cagaatctcactctgcccattctgtcggcgagacaggagtgcagtggtgtgatctcccctcacagcaacctctgcctccc | |
| | aggctatagtgattctcctgcctcagcctcctgagtagctggaattataggcgtgcgccactaccacctggctaattttt | |
| | gtatttttagtagagacagggtttcatcatgttgaccaggctagtcttaaactcctgacctcaaatgatatacctgcctt | |
| | ggcctcccgaagtgctggaattacaagtgtgagccaccgagcccagcagacacttttcttttttctttttttttttttga | |
| | gacagagtctcgcactgtcacccaggctggagtgcagtggcacaatctcagctcactgcaacctccacctcccgggttca | |
| | ggtgattctcctgtctcagcctctcgagtacctgggattacaggtgcctgccaccacgcccggctaattttttgtatttt | |
| | tagtagagacagggtttcactatgttggccaggatgattgcgaactcctgacctcgtgatctgcccacatcggcctccca | |
| | aagtgctgggattacatgcgtgagccactgacacttttctttgcccttttttggaccctgacttctgcccatccctgaca | |
| | tttggttcctgttttaatgccctgtgaaataagatttcaccgcctatcatctgctaactgctacggactcaggctcagaa | |
| | aggcctgcgcttcacccaggtgccagcctccacaggttccaacccaggagcccaagttccctttggccctgactcagaca | |
| | ctattaggactggcaagtgataagcagagtcccatactctcctattgactcggactaccatatcttgatcatccttttct | |
| | gtag | |
| Int005 | gtaggcattctagctttttcaggccctgagggccctgatgtctgggggttgagaaactgtagggtaggtctgcttgtaca | 28 |
| | gacattttgtcccctgctgttttgtcctggggggggagggtggaggctaatggctgaaccggatgcactggttgggctag | |
| | tatgtgttccaactctgggtgcttctctcttcactacctttgtctctag | |
| Int006 | gtaagggatcagcaaggatgtgggatcaggactggcctcccatttagccatgctgatctgtgtcccaaccctcaacctag | 29 |
| | ttccacttccagatctgcctgtcctcagctcacctttctaccttctgggcctttcagccttgggcctgtcaatcttgccc | |
| | actccatcaggcttcctgttctctcggtctggcccactttctttttatttttcttcttttttttttttttgagaaggagt | |
| | ctctctctctgtcacccaggctggagtgctgtggcgccatcttcactcactgtaacctctgcctcctgagttcaagcaat | |
| | tctcctgcctcagccttccaagtagctgggattataggcgcctgccaccaggcccagctgatttttctatttttagtaga | |
| | gacggggtttcgccaggctgttctcgaactcctgaactcaagtgatccacctgcctcggcttcccaaagtgctgggatta | |
| | caggtgtgagccaccacacccagctggtctggtccactttcttggccggatcattcatgacctttctcttgccag | |
| Int007 | gtaaggcctggacctccatggtgctccagtgaccttcaaatccagcatccaaatgactggctcccaaacttagagcgatt | 30 |
| | tctctacccaactatggattcctagagcaccattcccctggacctccagggtgccatggatcccacagttgtogcttgaa | |
| | acctttctaggggctgggcgaggtggctcactcatgcaaacccagcactttgggaagccgaggcgggtgatcacctgagg | |
| | tcaggagtttaagaccaccctggccaacgtgttgaaaccctgtgtctactaaaatacaaaaaaaaaaaattatctgggca | |
| | tgatggtgggtgtctgtaatcccagctactcaggaggctgagaagggagaatcagttgaacccgggagatggtggttgcg | |
| | gtgagccgagatcgcgccactgcactccagcctgggaggctgagcgagactccatctcgaaacaaaacaaaacaaaacta | |
| | tctaggctgggggtggtggttcatgtatgtatgtgtatatacatatatatgtgtttatatgtatatatatatacacacac | |
| | acacatacatacacacacatacacacacaaattagctgggtgtggcacccgtgtagtcccagctactcaggaggctaatg | |
| | tgggaggatcagttgaccctaggaagtcaaggctgcagtgagtcgtgattgcgccactgtactccagcccgagtgacaga | |
| | gtgacatcctgtctcaaaaaaaaaaaaaatctccccaaacctctctagttgcattcttcccgtcacccaactccaggatt | |
| | cctacaacaggaactagaagttccagaagcctgtgtgcaaggtccaggatcagttgctcttcctttgcag | |
| Int008 | gtaagccaccccagtctcccttcctgcaaagcagacctcagacctcttactagtttcaccaaagactgacagaagccctt | 31 |
| | cctgtccagctttccccagctagcctgcccttttgagcaactctggggaaccatgattccctatcttccctttccttcac | |
| | aggtctgcacacctcattgccccttttgcaactactgaggcacttgcagctgcctcagacttctcagctccccttgagat | |
| | gcctggatottcacacccccaactccttagctactaaggaatgtgcccctcacagggctgacctacccacagctgcctct | |
| | cccacatgtgacccttacctacactctctggggacccccagtgttgcgcctttgtctctttgcctttgtccttaccctag | |
| Int009 | gtgagtggaggggggcacccccattccataccaggcctatcatctcctacatcggatggcttacatcactctacaccacg | 32 |
| | agggagcaggaaggtgttcagggtggaacctoggaagaggcacacccatccccttttgcaccatggaggcaggaagtgac | |
| | taggtagcaacagaaaaccccaatgcctgaggctggactgcgatgcagaaaagcagggtcagtgcccagcagcatggctc | |
| | caggcctagagagccagggcagagcctctgcaggagttatggggtgggtccgtgggtgggtgacttcttagatgagggtt | |
| | tcatgggaggtaccccgagggactctgaccatctgttcccacattcag | |
| Int010 | gtgagggcaatggtgaggtctgggaagtgggctgaagacagcgttgggggccttggcaggatcacactctcagcttctcc | 33 |
| | tccctgctccctag | |
| Int011 | gtaagtatcaaggttacaagacaggtttaaggagaccaatagaaactgggcttgtcgagacagagaagactcttgcgttt | 34 |
| | ctgataggcacctattggtcttactgacatccactttgcctttctctccacag | |
| Int012 | gtaagtaccgcctatagactctataggcacacccctttggctcttatgcatgctatactgtttttggcttggggcctata | 35 |
| | cacccccgctccttatgctataggtgatggtatagcttagcctataggtgtgggttattgaccattattgaccactcccc | |
| | tattggtgacgatactttccattactaatccataacatggctctttgccacaactatctctattggctatatgccaatac | |
| | tctgtccttcagagactgacacggactctgtatttttacaggatggggtcccatttattatttacaaattcacatataca | |
| | acaacgccgtcccccgtgcccgcagtttttattaaacatagcgtgggatctccacgcgaatctcgggtacgtgttccgga | |
| | catgggctcttctccggtagcggoggagcttccacatccgagccctggtcccatgcctccagcggctcatggtcgctcgg | |
| | cagctccttgctcctaacagtggaggccagacttaggcacagcacaatgcccaccaccaccagtgtgccgcacaaggccg | |
| | tggcggtagggtatgtgtctgaaaatgagctcggagattgggctcgcaccgtgacgcagatggaagacttaaggcagcgg | |
| | cagaagaagatgcaggcagctgagttgttgtattctgataagagtcagaggtaactcccgttgcggtgctgttaacggtg | |
| | gagggcagtgtagtctgagcagtactcgttgctgccgcgcgcgccaccagacataatagctgacagactaacagactgtt | |
| | cctttccatgggtcttttctgcag | |
| Int013 | tcctcactctcttccgcatcgctgtctgcgagggccagctgttgggctcgcggttgaggacaaactcttcgcggtctttc | 36 |
| | cagtactcttggatoggaaacccgtcggcctccgaacggtactccgccaccgagggacctgagcgagtccgcatcgaccg | |
| | gatoggaaaacctctcgagaaaggcgtctaaccagtcacagtcgcaaggtaggctgagcaccgtggcgggcggcagcggg | |
| | tggcggtcggggttgtttctggcggaggtgctgctgatgatgtaattaaagtaggcggtcttgagacggcggatggtcga | |
| | ggtgaggtgtggcaggcttgagatccagctgttggggtgagtactccctctcaaaagcgggcattacttctgcgctaaga | |
| | ttgtcagtttccaaaaacgaggaggatttgatattcacctggcccgatctggccatacacttgagtgacaatgacatcca | |
| | ctttgcctttctctccacaggtgtccactcccaggtccaa | |
| Int014 | ctctaaggtaaatataaaatttttaagtgtataatgtgttaaactactgattctaattgtttctctcttttagattccaa | 37 |
| | cctttggaactga | |
| Int015 | gtaagtttagtctttttgtcttttatttcaggtcccggatccggtggtggtgcaaatcaaagaactgctcctcagtggat | 38 |
| | gttgcctttacttctaggc | |
| Int016 | aagaggtaagggtttaagggatggttggttggtggggtattaatgtttaattacctggagcacctgcctgaaatcacttt | 39 |
| | ttttcaggttgg | |
| Int017 | gtacgatggcgcctccagctaaaagagctaaaagaggtaagggtttaagggatggttggttggtggggtattaatgttta | 40 |
| | attacctgttttacaggcctgaaatcacttggttttagg | |
| Int018 | ggatcgatcctgagaacttcagggtgagtttggggacccttgattgttctttctttttcgctattgtaaaattcatgtta | 41 |
| | tatggagggggcaaagttttcagggtgttgtttagaatgggaagatgtcccttgtatcaccatggaccctcatgataatt | |
| | ttgtttctttcactttctactctgttgacaaccattgtctcctcttattttcttttcattttctgtaactttttcgttaa | |
| | actttagcttgcatttgtaacgaatttttaaattcacttttgtttatttgtcagattgtaagtactttctctaatcactt | |
| | ttttttcaaggcaatcagggtatattatattgtacttcagcacagttttagagaacaattgttataattaaatgataagg | |
| | tagaatatttctgcatataaattctggctggcgtggaaatattcttattggtagaaacaactacatcctggtcatcatcc | |
| | tgcctttctctttatggttacaatgatatacactgtttgagatgaggataaaatactctgagtccaaaccgggcccctct | |
| | gctaaccatgttcatgccttcttctttttcctacagctcctgggcaacgtgctggttattgtgctgtctcatcattttgg | |
| | caaagaatt | |
| Int019 | gaattaattcgctgtctgcgagggccggctgttggggtgagtactccctctcaaaagcgggcatgacttctgcgctaaga | 42 |
| | ttgtcagtttccaaaaacgaggaggatttgatattcacctggcccgcggtgatgcctttgagggtggccgcgtccatctg | |
| | gtcagaaaagacaatctttttgttgtcaagcttgaggtgtggcaggcttgagatctggccatacacttgagtgacaatga | |
| | catccactttgcctttctctccacaggtgtccactcccaggtccaactgcaggtcg | |
| Int020 | gtgagtttggggacccttgattgttctttctttttcgctattgtaaaattcatgttatatggagggggcaaagttttcag | 43 |
| | ggtgttgtttagaatgggaagatgtcccttgtatcaccatggaccctcatgataattttgtttctttcactttctactct | |
| | gttgacaaccattgtctcctcttattttcttttcattttctgtaactttttcgttaaactttagcttgcatttgtaacga | |
| | atttttaaattcacttttgtttatttgtcagattgtaagtactttctctaatcacttttttttcaaggcaatcagggtat | |
| | attatattgtacttcagcacagttttagagaacaattgttataattaaatgataaggtagaatatttctgcatataaatt | |
| | ctggctggcgtggaaatattcttattggtagaaacaactacatcctggtcatcatcctgcctttctctttatggttacaa | |
| | tgatatacactgtttgagatgaggataaaatactctgagtccaaaccgggcccctctgctaaccatgttcatgccttctt | |
| | ctttttcctacag | |

TABLE 5
Constructs information

| Constructs ID | Regulatory element ID | Intron ID | Intron Location Description |
|---|---|---|---|
| PG058 | HSRE002 | / | / |
| PG059 | HSRE002 | Int001 | inserted between 27-28 bp of SEQ ID NO: 21 |
| PG060 | HSRE002 | Int002 | inserted between 115-116 bp of SEQ ID NO: 21 |
| PG061 | HSRE002 | Int003 | inserted between 307-308 bp of SEQ ID NO: 21 |
| PG062 | HSRE002 | Int004 | inserted between 454-455 bp of SEQ ID NO: 21 |
| PG063 | HSRE002 | Int005 | inserted between 588-589 bp of SEQ ID NO: 21 |
| PG064 | HSRE002 | Int006 | inserted between 761-762 bp of SEQ ID NO: 21 |
| PG065 | HSRE002 | Int007 | inserted between 999-1000 bp of SEQ ID NO: 21 |
| PG066 | HSRE002 | Int008 | inserted between 1224-1225 bp of SEQ ID NO: 21 |
| PG067 | HSRE002 | Int009 | inserted between 1388-1389 bp of SEQ ID NO: 21 |
| PG068 | HSRE002 | Int010 | inserted between 1505-1506 bp of SEQ ID NO: 21 |
| PG074 | HSRE015 | / | / |
| PG075 | HSRE015 | Int012 | inserted between promoter and GBA1coding sequence |
| PG076 | HSRE015 | Int013 | inserted between promoter and GBA1 coding sequence |
| PG077 | HSRE015 | Int014 | inserted between promoter and GBA1 coding sequence |
| PG078 | HSRE015 | Int015 | inserted between promoter and GBA1 coding sequence |
| PG079 | HSRE015 | Int016 | inserted between promoter and GBA1 coding sequence |
| PG080 | HSRE015 | Int017 | inserted between promoter and GBA1 coding sequence |
| PG081 | HSRE015 | Int011 | inserted between promoter and GBA1 coding sequence |
| PG082 | HSRE016 | / | / |
| PG083 | HSRE016 | Int012 | inserted between promoter and GBA1 coding sequence |
| PG084 | HSRE016 | Int013 | inserted between promoter and GBA1 coding sequence |
| PG085 | HSRE016 | Int014 | inserted between promoter and GBA1 coding sequence |
| PG086 | HSRE016 | Int015 | inserted between promoter and GBA1 coding sequence |
| PG087 | HSRE016 | Int016 | inserted between promoter and GBA1 coding sequence |
| PG088 | HSRE016 | Int017 | inserted between promoter and GBA1 coding sequence |
| PG089 | HSRE016 | Int018 | inserted between promoter and GBA1 coding sequence |
| PG090 | HSRE016 | Int019 | inserted between promoter and GBA1 coding sequence |
| PG091 | HSRE016 | Int011 | inserted between promoter and GBA1 coding sequence |
| PG092 | HSRE001 | / | / |
| PG093 | HSRE001 | Int012 | inserted between promoter and GBA1 coding sequence |
| PG094 | HSRE001 | Int013 | inserted between promoter and GBA1 coding sequence |
| PG095 | HSRE001 | Int014 | inserted between promoter and GBA1 coding sequence |
| PG096 | HSRE001 | Int015 | inserted between promoter and GBA1 coding sequence |
| PG097 | HSRE001 | Int016 | inserted between promoter and GBA1 coding sequence |
| PG098 | HSRE001 | Int017 | inserted between promoter and GBA1 coding sequence |
| PG099 | HSRE001 | Int018 | inserted between promoter and GBA1 coding sequence |
| PG100 | HSRE001 | Int019 | inserted between promoter and GBA1 coding sequence |
| PG101 | HSRE001 | Int011 | inserted between promoter and GBA1 coding sequence |

TABLE 6
Codon-optimized GBA1 nucleotide sequence with the intron

| Name | Description | Nucleotide sequence (underline is intron sequence) | SEQ ID NO |
|---|---|---|---|
| mGBAil-C110 | Codon-optimized GBA1 nucleotide | atggagttctcaagtccttctcgcgaggtaagaagagagctcccaatcagcattgtcacagtgcttctgga | 44 |
| | sequence with Int001 inserted between | atcctggcactggaatttaatgaatgacagactctctttgaatccagggccatcatggctctttgagcaaggc | |
| | 27-28 bp of SEQ ID NO: 21 | acagatggagggaggggtcgaagttgaaatggggggaagagtggtggggagcatcctgatttggggtg | |
| | | ggcagagagttgtcatcagaagggttgcagggagagctgcacccaggtttctgtgggccttgtcctaatga | |
| | | atgtgggagaccgggccatgggcacccaaaggcagctaagccctgcccaggagagtagttgaggggtg | |
| | | gagaggggcttgcttttcagtcattcctcattctgtcctcaggaatgtcccaaaccactgagccgogtgtcaa | |
| | | ttatggctggatctctgaccggactgctgctgctgcaggccgtgagttgggctagtggggcccggccctgc | |
| | | atcccaaagagctttggctacagctctgtggtgtgcgtgtgcaacgccacctactgcgacagtttcgaccct | |
| | | cccaccttcccagccctgggcacattctctcggtacgagtccactcgctcaggccggagaatggagctgtc | |
| | | tatgggcccaatccaggctaatcataccggaacaggcctgctgctgacactgcagccagagcagaagttt | |
| | | cagaaggtgaaaggcttcgggggcgccatgaccgatgctgctgccctgaatatcctggccctgagtcccc | |
| | | cagctcagaatctgctgctgaagtcatactttagtgaggaaggcattggatacaatattatccgggtgcctat | |
| | | ggcctcctgtgacttctcaattcgcacctatacttacgctgatacccccgatgatttccagctgcacaacttca | |
| | | gtctgcccgaggaagataccaaactgaaaatcccactgatccatcgggctctgcagctggctcagcgccc | |
| | | agtgagcctgctggcctctccttggacctcaccaacatggctgaagactaacggcgccgtgaacgggaaa | |
| | | ggcagtctgaaagggcagccaggcgacatctatcaccagacttgggcccggtacttcgtgaaatttctgga | |
| | | tgcctacgctgagcacaaactgcagttttgggccgtgaccgccgaaaatgagccatcagccggcctgctg | |
| | | tctggctacccatttcagtgtctggggtttacaccagagcatcagagggacttcatcgcccgggacctggg | |
| | | accaaccctggccaacagcacccaccataacgtgaggctgctgatgctggacgatcagcggctgctgct | |
| | | gcctcactgggctaaggtggtgctgaccgatccagaggctgccaagtacgtgcatggaatcgccgtgcac | |
| | | tggtacctggatttcctggcccctgctaacgccaccctgggagagacccatcggctgttccccaacaccat | |
| | | gctgtttgcctctgaagcttgcgtggggtcaaagttttgggagcagtcagtgaggctgggcagttgggatcg | |
| | | ggggatgcagtactcccacagtattatcaccaatctgctgtaccatgtggtgggctggaccgattggaatct | |
| | | ggccctgaatccagaggggggccctaattgggtgcggaacttcgtggactccccaatcattgtggatatta | |
| | | caaaggacaccttctacaaacagcccatgttctaccacctgggccactttagtaagttcatccctgaggggt | |
| | | cacagcggggggcctggtggcctcccagaaaaacgacctggatgctgtggctctgatgcaccctgacg | |
| | | gatcagccgtggtggtggtgctgaatcgctctagtaaggacgtgccactgaccatcaaggatccagctgtg | |
| | | ggcttcctggagacaatcagtcccgggtactctatccacacctatctgtggcgccggcagtga | |

| | | | |
|---|---|---|---|
| mGBAi2-C110 | Codon-optimized GBA1 nucleotide | atggagttctcaagtccttctcgcgaggaatgtcccaaaccactgagccgcgtgtcaattatggctggatctc | 45 |
| | sequence with Int002 inserted between | tgaccggactgctgctgctgcaggccgtgagttgggctagtggtgagtgagtcaaggcagtggggaggt | |
| | 115-116 bp of SEQ ID NO: 21 | agcacagagcctcccttctgcctcatagtcctttggtagccttccagtaagctggtggtagacttttagtaggt | |
| | | gctcaataaatccttttgagtgactgagaccaactttggggtgaggattttgttttttttcttttgaaacagagtctt | |
| | | actctgttgcctgggctggagtgcagtggtgcaattttggctcattccaacctctgcctoccagattcaagog | |
| | | attctcttgcttcagcttcccaggtagctgggattacaggcggccaccactacgcccagctaatttttgtattttt | |
| | | agtagagacggggtttcaccatgctggcaaggcaggtctcaaactcctcacctcaggtgatccgcccacct | |
| | | cggcctcctaaagtgctaggattacaggtgtgagcccctgcgcccggccaaggggtgaggaattttgaaa | |
| | | ccgtgttcagtctctcctagcagatgtgtccattctccatgtcttcatcagacctcactctgcttgtactccctcc | |
| | | ctcccaggggcccggccctgcatcccaaagagctttggctacagctctgtggtgtgcgtgtgcaacgcca | |
| | | cctactgcgacagtttcgaccctcccaccttcccagccctgggcacattctctcggtacgagtccactcgct | |
| | | caggccggagaatggagctgtctatgggcccaatccaggctaatcataccggaacaggcctgctgctgac | |
| | | actgcagccagagcagaagtttcagaaggtgaaaggcttcgggggcgccatgaccgatgctgctgccct | |
| | | gaatatcctggccctgagtcccccagctcagaatctgctgctgaagtcatactttagtgaggaaggcattgg | |
| | | atacaatattatccgggtgcctatggcctcctgtgacttctcaattcgcacctatacttacgctgatacccccg | |
| | | atgatttccagctgcacaacttcagtctgcccgaggaagataccaaactgaaaatcccactgatccatoggg | |
| | | ctctgcagctggctcagcgcccagtgagcctgctggcctctccttggacctcaccaacatggctgaagact | |
| | | aacggcgccgtgaacgggaaaggcagtctgaaagggcagccaggcgacatctatcaccagacttgggc | |
| | | ccggtacttcgtgaaatttctggatgcctacgctgagcacaaactgcagttttgggccgtgaccgccgaaaa | |
| | | tgagccatcagccggcctgctgtctggctacccatttcagtgtctggggtttacaccagagcatcagaggg | |
| | | acttcatcgcccgggacctgggaccaaccctggccaacagcacccaccataacgtgaggctgctgatgct | |
| | | ggacgatcagcggctgctgctgcctcactgggctaaggtggtgctgaccgatccagaggctgccaagtac | |
| | | gtgcatggaatcgccgtgcactggtacctggatttcctggcccctgctaacgccaccctgggagagaccc | |
| | | atcggctgttccccaacaccatgctgtttgcctctgaagcttgcgtggggtcaaagttttgggagcagtcagt | |
| | | gaggctgggcagttgggatcgggggatgcagtactcccacagtattatcaccaatctgctgtaccatgtggt | |
| | | gggctggaccgattggaatctggccctgaatccagaggggggccctaattgggtgcggaacttcgtggac | |
| | | tccccaatcattgtggatattacaaaggacaccttctacaaacagcccatgttctaccacctgggccacttta | |
| | | gtaagttcatccctgaggggtcacagcgggtgggcctggtggcctoccagaaaaacgacctggatgctgt | |
| | | ggctctgatgcaccctgacggatcagccgtggtggtggtgctgaatcgctctagtaaggacgtgccactga | |
| | | ccatcaaggatccagctgtgggcttcctggagacaatcagtcccgggtactctatccacacctatctgtggc | |
| | | gccggcagtga | |
| mGBAi3-C110 | Codon-optimized GBA1 nucleotide | atggagttctcaagtccttctcgogaggaatgtcccaaaccactgagccgcgtgtcaattatggctggatctc | 46 |
| | sequence with Int003 inserted between | tgaccggactgctgctgctgcaggccgtgagttgggctagtggggcccggccctgcatcccaaagagctt | |
| | 307-308 bp of SEQ ID NO: 21 | tggctacagctctgtggtgtgcgtgtgcaacgccacctactgcgacagtttcgaccctcccaccttcccagc | |
| | | cctgggcacattctctcggtacgagtccactcgctcaggccggagaatggagctgtctatgggcccaatcc | |
| | | aggctaatcataccggaacaggtaaccattacacccctcaccccctgggccaggctgggtcctcctagag | |
| | | gtaaatggtgtcagtgatcaccatggagtttcccgctgggtactgatacccttattccctgtggatgtcctcag | |
| | | gcctgctgctgacactgcagccagagcagaagtttcagaaggtgaaaggcttcgggggcgccatgaccg | |
| | | atgctgctgccctgaatatcctggccctgagtcccccagctcagaatctgctgctgaagtcatactttagtga | |
| | | ggaaggcattggatacaatattatccgggtgcctatggcctcctgtgacttctcaattcgcacctatacttacg | |
| | | ctgatacccccgatgatttccagctgcacaacttcagtctgcccgaggaagataccaaactgaaaatocca | |
| | | ctgatccatogggctctgcagctggctcagcgcccagtgagcctgctggcctctccttggacctcaccaac | |
| | | atggctgaagactaacggcgccgtgaacgggaaaggcagtctgaaagggcagccaggcgacatctatc | |
| | | accagacttgggcccggtacttcgtgaaatttctggatgcctacgctgagcacaaactgcagttttgggccg | |
| | | tgaccgccgaaaatgagccatcagccggcctgctgtctggctacccatttcagtgtctggggtttacaccag | |
| | | agcatcagagggacttcatcgcccgggacctgggaccaaccctggccaacagcacccaccataacgtga | |
| | | ggctgctgatgctggacgatcagcggctgctgctgcctcactgggctaaggtggtgctgaccgatccaga | |
| | | ggctgccaagtacgtgcatggaatcgccgtgcactggtacctggatttcctggcccctgctaacgccaccc | |
| | | tgggagagacccatcggctgttccccaacaccatgctgtttgcctctgaagcttgcgtggggtcaaagttttg | |
| | | ggagcagtcagtgaggctgggcagttgggatcgggggatgcagtactcccacagtattatcaccaatctg | |
| | | ctgtaccatgtggtgggctggaccgattggaatctggccctgaatccagaggggggccctaattgggtgc | |
| | | ggaacttcgtggactccccaatcattgtggatattacaaaggacaccttctacaaacagcccatgttctacca | |
| | | cctgggccactttagtaagttcatccctgaggggtcacagcgggtgggcctggtggcctcccagaaaaac | |
| | | gacctggatgctgtggctctgatgcaccctgacggatcagccgtggtggtggtgctgaatcgctctagtaa | |
| | | ggacgtgccactgaccatcaaggatccagctgtgggcttcctggagacaatcagtcccgggtactctatcc | |
| | | acacctatctgtggcgccggcagtga | |
| mGBAi4-C110 | Codon-optimized GBA nucleotide | atggagttctcaagtccttctcgcgaggaatgtcccaaaccactgagccgcgtgtcaattatggctggatctc | 47 |
| | sequence with Int004 inserted between | tgaccggactgctgctgctgcaggccgtgagttgggctagtggggcccggccctgcatcccaaagagctt | |
| | 454-455 bp of SEQ ID NO: 21 | tggctacagctctgtggtgtgcgtgtgcaacgccacctactgcgacagtttcgaccctcccaccttcccagc | |
| | | cctgggcacattctctcggtacgagtccactcgctcaggccggagaatggagctgtctatgggcccaatcc | |
| | | aggctaatcataccggaacaggcctgctgctgacactgcagccagagcagaagtttcagaaggtgaaag | |
| | | gcttcgggggcgccatgaccgatgctgctgccctgaatatcctggccctgagtcccccagctcagaatctg | |
| | | ctgctgaagtcatactttagtgaggaaggtgaggaggaaggggacaagatgacatagagccattgaaact | |
| | | tttcgtttttcttttctttttttaaaatttttttgaggcagaatctcactctgcccattctgtcggcgagacaggagtg | |
| | | cagtggtgtgatctcccctcacagcaacctctgcctcccaggctatagtgattctcctgcctcagcctcctga | |
| | | gtagctggaattataggcgtgcgccactaccacctggctaatttttgtatttttagtagagacagggtttcatca | |
| | | tgttgaccaggctagtcttaaactcctgacctcaaatgatatacctgccttggcctcccgaagtgctggaatta | |
| | | caagtgtgagccaccgagcccagcagacacttttcttttttctttttttttttttgagacagagtctcgcactgtca | |
| | | cccaggctggagtgcagtggcacaatctcagctcactgcaacctccacctcccgggttcaggtgattctcct | |
| | | gtctcagcctctcgagtacctgggattacaggtgcctgccaccacgcccggctaattttttgtatttttagtaga | |
| | | gacagggtttcactatgttggccaggatgattgcgaactcctgacctcgtgatctgcccacatcggcctccc | |
| | | aaagtgctgggattacatgcgtgagccactgacacttttctttgccctttctttggaccctgacttctgcccatc | |
| | | cctgacatttggttcctgttttaatgccctgtgaaataagatttcaccgcctatcatctgctaactgctacggact | |
| | | caggctcagaaaggcctgcgcttcacccaggtgccagcctccacaggttccaacccaggagcccaagtt | |
| | | ccctttggccctgactcagacactattaggactggcaagtgataagcagagtcccatactctcctattgactc | |
| | | ggactaccatatettgatcatccttttctgtaggcattggatacaatattatccgggtgcctatggcctcctgtg | |
| | | acttctcaattcgcacctatacttacgctgatacccccgatgatttccagctgcacaacttcagtctgcccgag | |
| | | gaagataccaaactgaaaatcccactgatccatogggctctgcagctggctcagcgcccagtgagcctgc | |
| | | tggcctctccttggacctcaccaacatggctgaagactaacggcgccgtgaacgggaaaggcagtctgaa | |
| | | agggcagccaggcgacatctatcaccagacttgggcccggtacttcgtgaaatttctggatgcctacgctg | |
| | | agcacaaactgcagttttgggccgtgacegccgaaaatgagccatcagccggcctgctgtetggctaccc | |
| | | atttcagtgtctggggtttacaccagagcatcagagggacttcategcccgggacctgggaccaaccctgg | |
| | | ccaacagcacccaccataacgtgaggctgctgatgctggacgatcageggctgctgctgcctcactgggc | |
| | | taaggtggtgctgaccgatccagaggctgccaagtacgtgcatggaatcgccgtgcactggtacctggatt | |
| | | tcctggcccctgctaacgccaccctgggagagacccateggetgttccccaacaccatgtgtttgcctctg | |
| | | aagcttgcgtggggtcaaagttttgggagcagtcagtgaggctgggcagttgggatcgggggatgcagta | |
| | | ctcccacagtattatcaccaatctgctgtaccatgtggtgggctggaccgattggaatctggccctgaatcca | |
| | | gaggggggccctaattgggtgcggaacttegtggactccccaatcattgtggatattacaaaggacacctt | |
| | | ctacaaacagcccatgttctaccacctgggccactttagtaagttcatccctgaggggtcacagcgggtgg | |
| | | gcctggtggcctcccagaaaaacgacctggatgctgtggctctgatgcaccctgacggatcagccgtggt | |
| | | ggtggtgctgaatcgctctagtaaggacgtgccactgaccatcaaggatccagctgtgggcttcctggaga | |
| | | caatcagtcccgggtactctatccacacctatctgtggcgccggcagtga | |
| mGBAi5-C110 | Codon-optimized GBA1 nucleotide | atggagttctcaagtccttctegcgaggaatgtcccaaaccactgagccgcgtgtcaattatggctggatctc | 48 |
| | sequence with Int005 inserted between | tgaccggactgctgctgctgcaggccgtgagttgggctagtggggcccggccctgcatcccaaagagctt | |
| | 588-589 bp of SEQ ID NO: 21 | tggctacagctctgtggtgtgcgtgtgcaacgccacctactgegacagtttegaccctcccaccttcccagc | |
| | | cctgggcacattctcteggtacgagtccactegctcaggccggagaatggagctgtctatgggcccaatcc | |
| | | aggctaatcataccggaacaggcctgctgctgacactgcagccagagcagaagtttcagaaggtgaaag | |
| | | gcttcgggggcgccatgaccgatgctgctgccctgaatatcctggccctgagtcccccagctcagaatctg | |
| | | ctgctgaagtcatactttagtgaggaaggcattggatacaatattatccgggtgcctatggcctcctgtgactt | |
| | | ctcaattcgcacctatacttacgctgatacccccgatgatttccagctgcacaacttcagtctgcccgaggaa | |
| | | gataccaaactgaaagtaggcattctagctttttcaggccctgagggccctgatgtctgggggttgagaaac | |
| | | tgtagggtaggtctgcttgtacagacattttgtcccctgctgttttgtcctgggggtgggagggtggaggcta | |
| | | atggctgaaccggatgcactggttgggctagtatgtgttecaactctgggtgettetetettcactacctttgtc | |
| | | tctagatcccactgatccategggctctgcagctggctcagcgcccagtgagcctgctggcctctccttgga | |
| | | cctcaccaacatggctgaagactaacggcgccgtgaacgggaaaggcagtctgaaagggcagccaggc | |
| | | gacatctatcaccagacttgggcccggtacttcgtgaaatttctggatgcctacgctgagcacaaactgcag | |
| | | ttttgggccgtgacegccgaaaatgagccatcagccggcctgctgtctggctacccatttcagtgtctgggg | |
| | | tttacaccagagcatcagagggacttcategcccgggacctgggaccaaccctggccaacagcacccac | |
| | | cataacgtgaggctgctgatgctggacgatcagcggctgctgctgcctcactgggctaaggtggtgctgac | |
| | | cgatccagaggctgccaagtacgtgcatggaatcgccgtgcactggtacctggatttectggcccctgcta | |
| | | acgccaccctgggagagacccateggctgttccccaacaccatgctgtttgcctctgaagcttgcgtgggg | |
| | | tcaaagttttgggagcagtcagtgaggctgggcagttgggatcgggggatgcagtactcccacagtattat | |
| | | caccaatctgctgtaccatgtggtgggctggaccgattggaatctggccctgaatccagaggggggccct | |
| | | aattgggtgcggaacttcgtggactccccaatcattgtggatattacaaaggacaccttctacaaacagccc | |
| | | atgttctaccacctgggccactttagtaagttcatccctgaggggtcacagcgggtgggcctggtggcctcc | |
| | | cagaaaaacgacctggatgctgtggctctgatgcaccctgacggatcagccgtggtggtggtgctgaatc | |
| | | gctctagtaaggacgtgccactgaccatcaaggatccagctgtgggcttcctggagacaatcagtcccgg | |
| | | gtactctatccacacctatctgtggcgccggcagtga | |
| mGBAi6-C110 | Codon-optimized GBA1 nucleotide | atggagttctcaagtccttctegegaggaatgtcccaaaccactgagccgcgtgtcaattatggctggatctc | 49 |
| | sequence with Int006 inserted between | tgaccggactgctgctgctgcaggccgtgagttgggctagtggggcccggccctgcatcccaaagagctt | |
| | 761-762 bp of SEQ ID NO: 21 | tggctacagctctgtggtgtgcgtgtgcaacgccacctactgegacagtttegaccctoccacetteccagc | |
| | | cctgggcacattctcteggtacgagtccactegctcaggccggagaatggagctgtctatgggcccaatcc | |
| | | aggctaatcataccggaacaggcctgctgctgacactgcagccagagcagaagtttcagaaggtgaaag | |
| | | gcttcgggggcgccatgaccgatgctgctgccctgaatatcctggccctgagtcccccagctcagaatctg | |
| | | ctgctgaagtcatactttagtgaggaaggcattggatacaatattatccgggtgcctatggcctcctgtgactt | |
| | | ctcaattcgcacctatacttacgctgatacccccgatgatttccagctgcacaacttcagtetgcccgaggaa | |
| | | gataccaaactgaaaatcccactgatccategggctctgcagctggctcagcgcccagtgagcctgctgg | |
| | | cctctccttggacctcaccaacatggctgaagactaacggcgccgtgaacgggaaaggcagtctgaaag | |
| | | ggcagccaggcgacatctatcaccagacttgggcccggtacttcgtgaagtaagggatcagcaaggatgt | |
| | | gggatcaggactggcctcccatttagccatgctgatctgtgtcccaaccctcaacctagttecacttccagat | |
| | | ctgcctgtcctcagctcacctttctaccttetgggcctttcagcettgggcctgtcaatettgcccactecatca | |
| | | ggcttcctgttctcteggtetggcccactttetttttatttttgagaaggagtctctctctctgt | |
| | | cacccaggctggagtgctgtggegccatcttcacteactgtaacctetgcctectgagttcaagcaattetcc | |
| | | tgcctcagcettecaagtagetgggattataggegcctgccaccaggcccagctgatttttctatttttagtag | |
| | | agacggggtttegccaggctgttctegaactcctgaactcaagtgatecacctgcctoggettoccaaagtg | |
| | | ctgggattacaggtgtgagccaccacacccagctggtctggtccactttettggcoggatcattcatgacctt | |
| | | tctettgccagatttctggatgcctacgctgagcacaaactgcagttttgggccgtgaccgccgaaaatgag | |
| | | ccatcagccggcctgctgtetggctacccatttcagtgtctggggtttacaccagagcatcagagggacttc | |
| | | atcgcccgggacctgggaccaaccctggccaacagcacccaccataacgtgaggctgctgatgctggac | |
| | | gatcagcggctgctgctgcctcactgggctaaggtggtgctgaccgatccagaggctgccaagtacgtgc | |
| | | atggaatcgccgtgcactggtacctggatttcctggcccctgctaacgccaccctgggagagacccateg | |
| | | gctgttccccaacaccatgctgtttgcctctgaagcttgcgtggggtcaaagttttgggagcagtcagtgag | |
| | | gctgggcagttgggatcgggggatgcagtactcccacagtattatcaccaatctgctgtaccatgtggtgg | |
| | | gctggaccgattggaatctggccctgaatccagaggggggccctaattgggtgcggaacttegtggactc | |
| | | cccaatcattgtggatattacaaaggacaccttctacaaacagcccatgttctaccacctgggccactttagt | |
| | | aagttcatccctgaggggtcacagcgggtgggcctggtggcctcccagaaaaacgacctggatgctgtg | |
| | | gctctgatgcaccctgacggatcagccgtggtggtggtgctgaatcgctctagtaaggacgtgccactgac | |
| | | catcaaggatccagctgtgggcttcctggagacaatcagtcccgggtactctatccacacctatctgtggcg | |
| | | ccggcagtga | |
| mGBAi7-C110 | Codon-optimized GBA1 nucleotide | atggagttctcaagtccttctegegaggaatgtcccaaaccactgagccgegtgtcaattatggctggatctc | 50 |
| | sequence with Int007 inserted between | tgaccggactgctgctgctgcaggccgtgagttgggctagtggggcccggccctgcatcccaaagagctt | |
| | 999-1000 bp of SEQ ID NO: 21 | tggctacagctctgtggtgtgcgtgtgcaacgccacctactgegacagtttegaccctcccaccttcccagc | |
| | | cctgggcacattctcteggtacgagtccactegctcaggccggagaatggagctgtctatgggcccaatcc | |
| | | aggctaatcataccggaacaggcctgctgctgacactgcagccagagcagaagtttcagaaggtgaaag | |
| | | gcttcgggggcgccatgaccgatgctgctgccctgaatatcctggccctgagtcccccagctcagaatctg | |
| | | ctgctgaagtcatactttagtgaggaaggcattggatacaatattatccgggtgcctatggcctcctgtgactt | |
| | | ctcaattcgcacctatacttacgctgatacccccgatgatttccagctgcacaacttcagtctgcccgaggaa | |
| | | gataccaaactgaaaatcccactgatccatogggctctgcagctggctcagcgcccagtgagcctgctgg | |
| | | cctctccttggacctcaccaacatggctgaagactaacggcgccgtgaacgggaaaggcagtctgaaag | |
| | | ggcagccaggcgacatctatcaccagacttgggcccggtacttcgtgaaatttctggatgcctacgctgag | |
| | | cacaaactgcagttttgggccgtgacegccgaaaatgagccatcagccggcctgctgtctggctacccatt | |
| | | tcagtgtctggggtttacaccagagcatcagagggacttcategcccgggacctgggaccaaccctggcc | |
| | | aacagcacccaccataacgtgaggctgctgatgctggacgatcageggctgctgctgcctcactgggcta | |
| | | aggtggtaaggcctggacctccatggtgctccagtgaccttcaaatccagcatccaaatgactggctecca | |
| | | aacttagagegatttetctacccaactatggattectagagcaccatteccctggacctccagggtgccatgg | |
| | | atcccacagttgtcgcttgaaacctttctaggggctgggcgaggtggctcactcatgcaaacccagcacttt | |
| | | gggaagccgaggcgggtgatcacctgaggtcaggagtttaagaccaccctggccaacgtgttgaaaccc | |
| | | tgtgtctactaaaatacaaaaaaaaaaaattatctgggcatgatggtgggtgtctgtaatcccagctactcag | |
| | | gaggctgagaagggagaatcagttgaacccgggagatggtggttgcggtgagccgagatcgcgccact | |
| | | gcactccagcctgggaggctgagcgagactccatctcgaaacaaaacaaacaaaactatctaggctgg | |
| | | gggtggtggttcatgtatgtatgtgtatatacatatatatgtgtttatatgtatatatatacacacacacacata | |
| | | catacacacatacacacacaaattagctgggtgtggcacccgtgtagtcccagctactcaggaggctaa | |
| | | tgtgggaggatcagttgaccctaggaagtcaaggctgcagtgagtcgtgattgcgccactgtactccagcc | |
| | | cgagtgacagagtgacatcctgtctcaaaaacaaaaaaaaatctccccaaacctctctagttgcattcttccc | |
| | | gtcacccaactccaggattcctacaacaggaactagaagttccagaagcctgtgtgcaaggtccaggatca | |
| | | gttgctcttcctttgcaggtgctgaccgatccagaggctgccaagtacgtgcatggaatcgccgtgcactgg | |
| | | tacctggatttcctggcccctgctaacgccaccctgggagagacccateggctgttccccaacaccatgct | |
| | | gtttgcctctgaagcttgcgtggggtcaaagttttgggagcagtcagtgaggctgggcagttgggatcggg | |
| | | ggatgcagtactcccacagtattatcaccaatctgctgtaccatgtggtgggctggaccgattggaatctgg | |
| | | ccctgaatccagaggggggccctaattgggtgcggaacttogtggactccccaatcattgtggatattacaa | |
| | | aggacaccttctacaaacagcccatgttctaccacctgggccactttagtaagttcatccctgaggggtcac | |
| | | agcgggtgggcctggtggcctcccagaaaaacgacctggatgtgtggctctgatgcaccctgacggatc | |
| | | agccgtggtggtggtgctgaatcgctctagtaaggacgtgccactgaccatcaaggatccagctgtgggct | |
| | | tcctggagacaatcagtcccgggtactctatccacacctatetgtggcgccggcagtga | |
| mGBAi8-C110 | Codon-optimized GBA1 nucleotide | atggagttctcaagtccttctegcgaggaatgtcccaaaccactgagccgcgtgtcaattatggctggatctc | 51 |
| | sequence with Int008 inserted between | tgaccggactgctgctgctgcaggccgtgagttgggctagtggggcccggccctgcatcccaaagagctt | |
| | 1224-1225 bp of SEQ ID NO: 21 | tggctacagctctgtggtgtgcgtgtgcaacgccacctactgegacagtttegaccctcccacetteccagc | |
| | | cctgggcacattctcteggtacgagtccactcgctcaggccggagaatggagctgtctatgggcccaatcc | |
| | | aggctaatcataccggaacaggcctgctgctgacactgcagccagagcagaagtttcagaaggtgaaag | |
| | | gcttcgggggcgccatgaccgatgctgctgccctgaatatcctggccctgagtcccccagctcagaatctg | |
| | | ctgctgaagtcatactttagtgaggaaggcattggatacaatattatccgggtgcctatggcctcctgtgactt | |
| | | ctcaattcgcacctatacttacgctgatacccccgatgatttccagctgcacaacttcagtctgcccgaggaa | |
| | | gataccaaactgaaaatcccactgatccatogggctctgcagctggctcagcgcccagtgagcctgctgg | |
| | | cctctccttggacctcaccaacatggetgaagactaacggcgccgtgaacgggaaaggcagtctgaaag | |
| | | ggcagccaggcgacatctatcaccagacttgggcccggtacttcgtgaaatttctggatgcctacgctgag | |
| | | cacaaactgcagttttgggccgtgaccgccgaaaatgagccatcagccggcctgctgtctggctacccatt | |
| | | tcagtgtctggggtttacaccagagcatcagagggacttcategcccgggacctgggaccaaccctggcc | |
| | | aacagcacccaccataacgtgaggctgctgatgctggacgatcagcggctgctgctgcctcactgggcta | |
| | | aggtggtgctgaccgatccagaggctgccaagtacgtgcatggaatcgccgtgcactggtacctggatttc | |
| | | ctggcccctgctaacgccaccctgggagagacccateggctgttccccaacaccatgctgtttgcctctga | |
| | | agcttgcgtggggtcaaagttttgggagcagtcagtgaggctgggcagttgggatcgggggatgcagtac | |
| | | tcccacagtattatcaccgtaagccaccccagtetecettectgcaaagcagacctcagacctcttactagttt | |
| | | caccaaagactgacagaagccettectgtecagettteccoctagcctgcccttttgagcaactctgggg | |
| | | aaccatgattccctatettccctttccttcacaggtctgcacacctcattgccccttttgcaactactgaggcact | |
| | | tgcagctgcctcagacttctcagetccccttgagatgectggatottcacacccccaactecttagetactaa | |
| | | ggaatgtgcccctcacagggctgacctacccacagetgcctetcccacatgtgaccettacctacactctct | |
| | | ggggacccccagtgttgcgcctttgtctctttgcctttgtccttaccctagaatctgctgtaccatgtggtgggc | |
| | | tggaccgattggaatctggccctgaatccagaggggggccctaattgggtgeggaacttegtggactccc | |
| | | caatcattgtggatattacaaaggacaccttctacaaacagcccatgttctaccacctgggccactttagtaa | |
| | | gttcatccctgaggggtcacagcgggtgggcctggtggcctcccagaaaaacgacctggatgctgtggct | |
| | | ctgatgcaccctgacggatcagccgtggtggtggtgctgaatcgctctagtaaggacgtgccactgaccat | |
| | | caaggatccagctgtgggcttcctggagacaatcagtccegggtactctatccacacctatetgtggegcc | |
| | | ggcagtga | |
| mGBAi9-C110 | Codon-optimized GBA1 nucleotide | atggagttctcaagtccttctegogaggaatgtcccaaaccactgagccgcgtgtcaattatggctggatctc | 52 |
| | sequence with Int009 inserted between | tgaccggactgctgctgctgcaggccgtgagttgggctagtggggcccggccctgcatcccaaagagctt | |
| | 1388-1389 bp of SEQ ID NO: 21 | tggctacagctctgtggtgtgcgtgtgcaacgccacctactgegacagtttegaccctcccacetteccagc | |
| | | cctgggcacattctcteggtacgagtccactegctcaggccggagaatggagctgtctatgggcccaatcc | |
| | | aggctaatcataccggaacaggcctgctgctgacactgcagccagagcagaagtttcagaaggtgaaag | |
| | | gcttcgggggcgccatgaccgatgctgctgccctgaatatcctggccctgagtcccccagctcagaatctg | |
| | | ctgctgaagtcatactttagtgaggaaggcattggatacaatattatccgggtgcctatggcctcctgtgactt | |
| | | ctcaattcgcacctatacttacgctgatacccccgatgatttccagctgcacaacttcagtctgcccgaggaa | |
| | | gataccaaactgaaaatcccactgatccategggctctgcagctggctcagcgcccagtgagcctgctgg | |
| | | cctctccttggacctcaccaacatggctgaagactaacggcgccgtgaacgggaaaggcagtctgaaag | |
| | | ggcagccaggcgacatctatcaccagacttgggcccggtacttcgtgaaatttctggatgcctacgctgag | |
| | | cacaaactgcagttttgggccgtgaccgccgaaaatgagccatcagccggcctgctgtetggctacccatt | |
| | | tcagtgtctggggtttacaccagagcatcagagggacttcategccegggacctgggaccaaccctggcc | |
| | | aacagcacccaccataacgtgaggctgctgatgctggacgatcagcggctgctgctgcctcactgggcta | |
| | | aggtggtgctgaccgatccagaggctgccaagtacgtgcatggaatcgccgtgcactggtacctggatttc | |
| | | ctggcccctgctaacgccaccctgggagagacccateggctgttccccaacaccatgctgtttgcctctga | |
| | | agcttgcgtggggtcaaagttttgggagcagtcagtgaggctgggcagttgggatcgggggatgcagtac | |
| | | tcccacagtattatcaccaatctgctgtaccatgtggtgggctggaccgattggaatctggccctgaatccag | |
| | | aggggggccctaattgggtgcggaacttcgtggactccccaatcattgtggatattacaaaggacaccttct | |
| | | acaaacagcccatgttctaccacctgggccactttaggtgagtggaggggggcacccccattccatacc | |
| | | aggcctatcatctcctacatcggatggcttacatcactctacaccacgagggagcaggaaggtgttcaggg | |
| | | tggaacctcggaagaggcacacccatccccttttgcaccatggaggcaggaagtgactaggtagcaaca | |
| | | gaaaaccccaatgcctgaggetggactgegatgcagaaaagcagggtcagtgcccagcagcatggctc | |
| | | caggcctagagagccagggcagagcctctgcaggagttatggggtoggtccgtgggtgggtgacttctta | |
| | | gatgagggtttcatgggaggtaccccgagggactctgaccatctgttcccacattcagtaagttcatccctg | |
| | | aggggtcacagcgggtgggcctggtggcctcccagaaaaacgacctggatgctgtggctctgatgcacc | |
| | | ctgacggatcagccgtggtggtggtgctgaatcgctctagtaaggacgtgccactgaccatcaaggatcca | |
| | | gctgtgggcttcctggagacaatcagtcccgggtactctatccacacctatctgtggcgccggcagtga | |
| mGBAi10-C110 | Codon-optimized GBA1 nucleotide | atggagttctcaagtccttctegcgaggaatgtcccaaaccactgagcegegtgtcaattatggctggatctc | 53 |
| | sequence with Int010 inserted between | tgaccggactgctgctgctgcaggccgtgagttgggctagtggggcccggccctgcatcccaaagagctt | |
| | 1505-1506 bp of SEQ ID NO: 21 | tggctacagctctgtggtgtgcgtgtgcaacgccacctactgegacagtttegaccctcccaccttcccagc | |
| | | cctgggcacattctcteggtacgagtccactegctcaggccggagaatggagctgtctatgggcccaatcc | |
| | | aggctaatcataccggaacaggcctgctgctgacactgcagccagagcagaagtttcagaaggtgaaag | |
| | | gcttcgggggcgccatgacegatgctgctgccctgaatatectggccctgagtcccccagctcagaatctg | |
| | | ctgctgaagtcatactttagtgaggaaggcattggatacaatattatccgggtgcctatggcctcctgtgactt | |
| | | ctcaattcgcacctatacttacgctgatacccccgatgatttccagctgcacaacttcagtctgcccgaggaa | |
| | | gataccaaactgaaaatcccactgatccategggctctgcagctggctcagcgcccagtgagcctgctgg | |
| | | cctctccttggacctcaccaacatggctgaagactaacggcgccgtgaacgggaaaggcagtctgaaag | |
| | | ggcagccaggcgacatctatcaccagacttgggcccggtacttcgtgaaatttctggatgcctacgctgag | |
| | | cacaaactgcagttttgggccgtgacegccgaaaatgagccatcagccggcctgctgtctggctacccatt | |
| | | tcagtgtctggggtttacaccagagcatcagagggacttcategccegggacctgggaccaaccctggcc | |
| | | aacagcacccaccataacgtgaggctgctgatgctggacgatcagcggctgctgctgcctcactgggcta | |
| | | aggtggtgctgaccgatccagaggctgccaagtacgtgcatggaatcgccgtgcactggtacctggatttc | |
| | | ctggcccctgctaacgccaccctgggagagacccateggctgttccccaacaccatgtgtttgcctctga | |
| | | agcttgcgtggggtcaaagttttgggagcagtcagtgaggctgggcagttgggatcgggggatgcagtac | |
| | | tcccacagtattatcaccaatctgctgtaccatgtggtgggctggaccgattggaatctggccctgaatccag | |
| | | aggggggccctaattgggtgcggaacttegtggactccccaatcattgtggatattacaaaggacaccttct | |
| | | acaaacagcccatgttctaccacctgggccactttagtaagttcatccctgaggggtcacagcgggtgggc | |
| | | ctggtggcctcccagaaaaacgacctggatgctgtggctctgatgcaccctgacggatcagccgtggtgg | |
| | | tggtgctgaatcggtgagggcaatggtgaggtctgggaagtgggctgaagacagcgttgggggccttgg | |
| | | caggatcacactctcagettetectccctgetccctagctctagtaaggacgtgccactgaccatcaaggat | |
| | | ccagctgtgggcttcctggagacaatcagtcccgggtactctatccacacctatctgtggcgccggcagtg | |
| | | a | |
| mGBAi1i5-C110 | Codon-optimized GBA1 nucleotide | atggagttctcaagtccttctcgcgaggtaagagagagagctcccaatcagcattgtcacagtgcttctgga | 54 |
| | sequence with Int001 inserted between | atcctggcactggaatttaatgaatgacagactctctttgaatccagggccatcatggetetttgagcaaggc | |
| | 27-28 bp and Int005 inserted between | acagatggagggaggggtcgaagttgaaatgggtoggaagagtggtggggagcatcctgatttggggtg | |
| | 588-589 bp of SEQ ID NO: 21 | ggcagagagttgtcatcagaagggttgcagggagagctgcacccaggtttctgtgggccttgtcctaatga | |
| | | atgtgggagaccgggccatgggcacccaaaggcagctaagccctgcccaggagagtagttgaggggtg | |
| | | gagaggggcttgcttttcagtcattcctcattetgtcctcaggaatgtcccaaaccactgagccgcgtgtcaa | |
| | | ttatggctggatctctgaccggactgctgctgctgcaggccgtgagttgggctagtggggcccggccctgc | |
| | | atcccaaagagctttggctacagctctgtggtgtgcgtgtgcaacgccacctactgegacagtttegaccct | |
| | | cccaccttcccagccctgggcacattetcteggtacgagtccactcgctcaggccggagaatggagctgtc | |
| | | tatgggcccaatccaggctaatcataccggaacaggcctgctgctgacactgcagccagagcagaagttt | |
| | | cagaaggtgaaaggcttcgggggcgccatgaccgatgctgctgccctgaatatcctggccctgagtcccc | |
| | | cagctcagaatctgctgctgaagtcatactttagtgaggaaggcattggatacaatattatccgggtgcctat | |
| | | ggcctcctgtgacttctcaattcgcacctatacttacgctgatacccccgatgatttccagctgcacaacttca | |
| | | gtctgcccgaggaagataccaaactgaaagtaggcattctagctttttcaggccctgagggccctgatgtct | |
| | | gggggttgagaaactgtagggtaggtctgcttgtacagacattttgtcccctgctgttttgtcctgggggtgg | |
| | | gagggtggaggctaatggctgaaccggatgcactggttgggctagtatgtgttccaactctgggtgcttctc | |
| | | tcttcactacctttgtetctagatcccactgatccategggctctgcagctggctcagegcccagtgagectg | |
| | | ctggcctctccttggacctcaccaacatggctgaagactaacggcgccgtgaacgggaaaggcagtctga | |
| | | aagggcagccaggcgacatctatcaccagacttgggcccggtacttegtgaaatttctggatgcctacgct | |
| | | gagcacaaactgcagttttgggccgtgacegccgaaaatgagccatcagccggcctgctgtctggctacc | |
| | | catttcagtgtctggggtttacaccagagcatcagagggacttcategcccgggacctgggaccaaccctg | |
| | | gccaacagcacccaccataacgtgaggctgctgatgctggacgatcagcggctgctgctgcctcactggg | |
| | | ctaaggtggtgctgaccgatccagaggctgccaagtacgtgcatggaatcgccgtgcactggtacctgga | |
| | | tttcctggcccctgctaacgccaccctgggagagacccateggetgttccccaacaccatgctgtttgcctct | |
| | | gaagcttgcgtggggtcaaagttttgggagcagtcagtgaggctgggcagttgggatcgggggatgcagt | |
| | | actcccacagtattatcaccaatctgctgtaccatgtggtgggctggacegattggaatctggccctgaatcc | |
| | | agaggggggccctaattgggtgcggaacttegtggactccccaatcattgtggatattacaaaggacacct | |
| | | tctacaaacagcccatgttctaccacctgggccactttagtaagttcatccctgaggggtcacagcgggtgg | |
| | | gcctggtggcctcccagaaaaacgacctggatgctgtggctctgatgcaccctgacggatcagccgtggt | |
| | | ggtggtgctgaatcgctctagtaaggacgtgccactgaccatcaaggatccagctgtgggcttcctggaga | |
| | | caatcagtccegggtactctatccacacctatetgtggegccggcagtga | |
| mGBAiC-C110 | Codon-optimized GBA1 nucleotide | gtaagtatcaaggttacaagacaggtttaaggagaccaatagaaactgggcttgtcgagacagagaagac | 55 |
| | sequence with chimeric intron | tcttgcgtttctgataggcacctattggtcttactgacatccactttgcctttctctccacaggccaccatggagt | |
| | inserted between 27-28 bp of SEQ ID NO: 21 | tctcaagtccttctegogaggaatgtcccaaaccactgagccgcgtgtcaattatggctggatctctgaccg | |
| | | gactgctgctgctgcaggccgtgagttgggctagtggggcccggccctgcatcccaaagagctttggcta | |
| | | cagctctgtggtgtgcgtgtgcaacgccacctactgegacagtttegacccteccaccttcccagccctgg | |
| | | gcacattctctoggtacgagtccactcgctcaggccggagaatggagctgtctatgggcccaatccaggct | |
| | | aatcataccggaacaggcctgctgctgacactgcagccagagcagaagtttcagaaggtgaaaggcttcg | |
| | | ggggcgccatgaccgatgctgctgccctgaatatcctggccctgagtcccccagctcagaatctgctgctg | |
| | | aagtcatactttagtgaggaaggcattggatacaatattatccgggtgcctatggcctcctgtgacttctcaatt | |
| | | cgcacctatacttacgctgatacccccgatgatttccagctgcacaacttcagtctgcccgaggaagatacc | |
| | | aaactgaaaatcccactgatecategggetctgcagctggctcagegcccagtgagcetgetggcctetcc | |
| | | ttggacctcaccaacatggctgaagactaacggcgccgtgaacgggaaaggcagtctgaaagggcagc | |
| | | caggcgacatctatcaccagacttgggcccggtacttogtgaaatttctggatgcctacgctgagcacaaac 55 | |
| | | tgcagttttgggccgtgaccgccgaaaatgagccatcagccggcctgctgtctggctacccatttcagtgtc | |
| | | tggggtttacaccagagcatcagagggacttcategcccgggacctgggaccaaccctggccaacagca | |
| | | cccaccataacgtgaggctgctgatgctggacgatcagcggctgctgctgcctcactgggctaaggtggt | |
| | | gctgaccgatccagaggctgccaagtacgtgcatggaatcgccgtgcactggtacctggatttcetggccc | |
| | | ctgctaacgccaccctgggagagacccateggetgttccccaacaccatgtgtttgcctctgaagcttgcg | |
| | | tggggtcaaagttttgggagcagtcagtgaggctgggcagttgggatcgggggatgcagtactcccacag | |
| | | tattatcaccaatctgctgtaccatgtggtgggctggaccgattggaatctggccctgaatccagagggggg | |
| | | ccctaattgggtgcggaacttogtggactccccaatcattgtggatattacaaaggacaccttctacaaacag | |
| | | cccatgttctaccacctgggccactttagtaagttcatccctgaggggtcacagcgggtgggcctggtggc | |
| | | ctcccagaaaaacgacctggatgctgtggctctgatgcaccctgacggatcagccgtggtggtggtgctg | |
| | | aatcgctctagtaaggacgtgccactgaccatcaaggatccagctgtgggcttcctggagacaatcagtcc | |
| | | cgggtactctatccacacctatctgtggogccggcagtga | |

### Example 4

In Vitro Testing of Chimeric HSREs with Intron

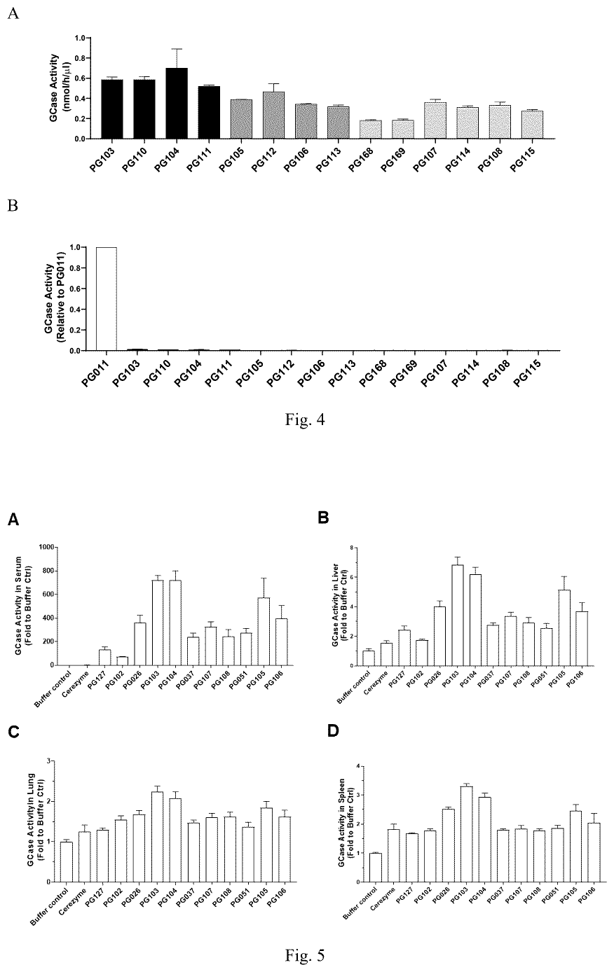

Constructs with endogenous GBA1 intron 1 (Int001, il) and a chimeric intron (Int011, iC) showed higher GCase expression when constructed with different CHSREs in HepG2 cells from the results described above, therefore, these two introns were cloned into the GCase-expressing cassettes comprising of CHSREs and mGBA-C110. Constructs with Int001 inserted between 27 bp and 28 bp of SEQ ID NO:21 of GBA1 are PG103, PG104, PG105, PG106, PG168, PG107, PG108. Constructs with exogenous chimeric intron inserted between chimeric HSREs and GBA1 coding sequence are PG110, PG111, PG112 and PG113, PG169, PG114 and PG115. Transfections were conducted on HepG2 and HEK293T cells, the culture supernatants were harvested for measuring GCase activity. The results showed that all these constructs with the combination of chimeric HSREs and both of endogenous intron 1 and exogenous chimeric intron expressed GCase efficiently in HepG2 cells GBA1 (FIG. 4A). All of these constructs showed very weak GCase activity in HEK293T cells compared to the PG011 construct with mGBA-C110 driven by a CRE001 promoter, which was used as a universal expression control (FIG. 4B).

TABLE 7
Construct information

| Constructs ID | Regulatory element ID | Intron ID | Intron Location |
|---|---|---|---|
| PG102 | HSRE002 | Int001 | inserted between 27-28 bp of SEQ ID NO: 21 |
| PG103 | CHSRE005 | Int001 | inserted between 27-28 bp of SEQ ID NO: 21 |
| PG104 | CHSRE002 | Int001 | inserted between 27-28 bp of SEQ ID NO: 21 |
| PG105 | CHSRE030 | Int001 | inserted between 27-28 bp of SEQ ID NO: 21 |
| PG106 | CHSRE027 | Int001 | inserted between 27-28 bp of SEQ ID NO: 21 |
| PG107 | CHSRE016 | Int001 | inserted between 27-28 bp of SEQ ID NO: 21 |
| PG108 | CHSRE013 | Int001 | inserted between 27-28 bp of SEQ ID NO: 21 |
| PG109 | HSRE002 | Int011 | inserted between promoter and GBA1 coding sequence |
| PG110 | CHSRE005 | Int011 | inserted between promoter and GBA1 coding sequence |
| PG111 | CHSRE002 | Int011 | inserted between promoter and GBA1 coding sequence |
| PG112 | CHSRE030 | Int011 | inserted between promoter and GBA1 coding sequence |
| PG113 | CHSRE027 | Int011 | inserted between promoter and GBA1 coding sequence |
| PG114 | CHSRE016 | Int011 | inserted between promoter and GBA1 coding sequence |
| PG115 | CHSRE013 | Int011 | inserted between promoter and GBA1 coding sequence |
| PG168 | HSRE005 | Int001 | inserted between 27-28 bp of SEQ ID NO: 21 |
| PG169 | HSRE005 | Int011 | inserted between promoter and GBA1 coding sequence |

### Example 5

In Vivo Study of Constructs with Chimeric HSREs and Introns in Wild Type Mice Using AAV8 Vector

According to the results described above, constructs of PG102, PG026, PG103, PG104, PG037, PG107, PG108, PG051, PG105 and PG106, were selected and packaged with AAV8 for further study in wild type mice. These AAV8 products along with reference product of PG127, which is constructed with the combination of sequence of SEQ ID NO: 14, SEQ ID NO: 5, and SEQ ID NO: 23 as described in patent WO2020161483A1, were injected into wild type mice at the dose of 2E12 vg/kg (FIG. 5). Buffer control group and enzyme replacement therapy (Cerezyme) group were also included in this study. The results showed that all of these AAV8 products efficiently increase GCase activity in serum, liver, spleen and lung. Enzyme activity results from serum were highly consistent with that from different tissue lysates. Most groups of AAV8 products of PG102, PG026, PG103, PG104, PG037, PG107, PG108, PG051, PG105 and PG106 performed much better or at least comparable with Cerezyme group.

In conclusion, the constructs with the combination of HSREs or chimeric HSREs and Int001 described herein, which were packaged by AAV8, specifically delivered GBA1 into the liver and increase the GCase activity systemically in serum and all other target tissues.

### Example 6

### In Vivo Study of Therapeutic Potential for AAV8 Candidates Against Gaucher Disease

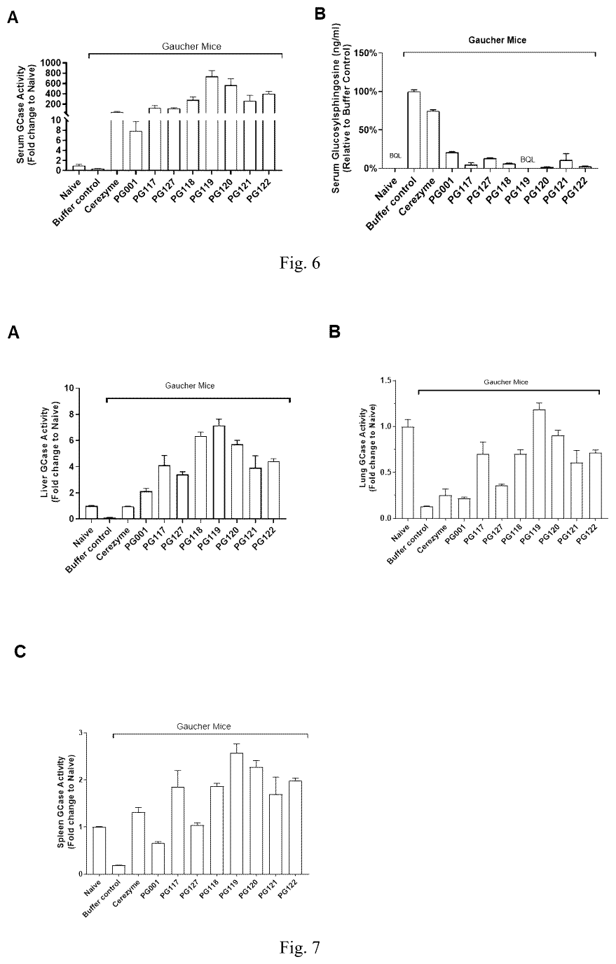

The constructs of PG011, PG103, PG104, PG107 and PG105 described above all carried the Ampicillin resistance, which were all replaced by Kanamycin and renamed as PG117, PG118, PG119, PG120, PG121 and PG122. In order to study the long-term therapeutic effect of these AAV8 candidates for gene therapy against Gaucher disease, constructs with CHSRE-driven mGBAi1-C110 of PG119, PG120, PG121 and PG122, constructs with CHSRE-driven GBAi1-C110 of PG118, and also the CRE001-driven mGBA-C110 of PG117, were packaged as AAV8. These AAV8 candidates along with wild type GBA1 control of PG001 and PG127 as the reference product were administered into Gaucher mice at dose of 2E12 vg/kg by tail vein injection and underwent a 12-weeks therapeutic study. Some control groups, including wild type control (Naive), buffer control and enzyme replacement therapy (Cerezyme) group were also included in this study. Serum enzyme activity and glucosylsphingosine accumulation were monitored at interval time point (week 1, 2, 4, 6, 8, and 12) post injection. According to the results, all AAV8 injected group showed high and stable GCase activity in serum. The AAV8 candidates of PG117, PG118, PG119, PG120, PG121 and PG122 showed higher GCase activity than the buffer control group, Cerezyme group and reference product of PG127 (FIG. 6A). The glucosylsphingosine level in serum decreased quickly after the injection of AAV8 products. AAV8 candidates of PG117, PG118, PG119, PG120, PG121 and PG122 decreased the glucosylsphingosine close to the level of naive wild type mice after 8 weeks post injection (FIG. 6B).

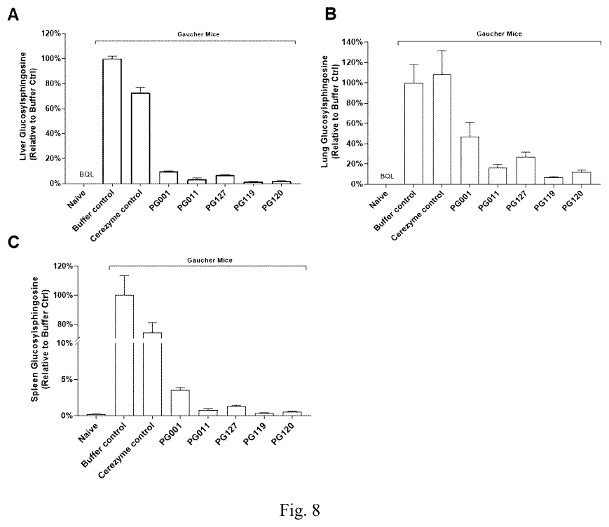

The tissue samples were collected at the end of the study for GCase enzyme activity and glucosylsphingosine analysis. The GCase enzyme activity were increased in liver, lung, and spleen for all AAV injected groups. Consistent with the results in serum described above, groups of AAV8 products of PG117, PG118, PG119, PG120, PG121 and PG122 showed higher GCase activity and lower glucosylsphingosine accumulation in all tested tissues than groups of Cerezyme and reference product of PG127 (FIG. 7 and FIG. 8).

In conclusion, AAV8 products of PG117, PG118, PG119, PG120, PG121 and PG122 were proven to be promising therapeutic candidates against Gaucher disease, and performed superior than the existing therapies of Cerezyme.

TABLE 8
Construct information

| Construct ID | Regulatory element ID | Intron ID | Intron Location |
|---|---|---|---|
| PG117 | CRE001 | / | / |
| PG118 | CHSRE005 | Int001 | inserted between 27-28 bp of SEQ ID NO: 21, and the |
| | | | nucleotide base of C at 1080 bp of SEQ ID NO: 21 is |
| | | | mutated to A |
| PG119 | CHSRE005 | Int001 | inserted between 27-28 bp of SEQ ID NO: 21 |
| PG120 | CHSRE002 | Int001 | inserted between 27-28 bp of SEQ ID NO: 21 |
| PG121 | CHSRE016 | Int001 | inserted between 27-28 bp of SEQ ID NO: 21 |
| PG122 | CHSRE030 | Int001 | inserted between 27-28 bp of SEQ ID NO: 21 |
| PG123 | CRE001 | Int001 | inserted between 27-28 bp of SEQ ID NO: 21 |
| PG124 | CRE001 | Int002 | inserted between 115-116 bp of SEQ ID NO: 21 |
| PG125 | CRE001 | Int005 | inserted between 588-589 bp of SEQ ID NO: 21 |
| PG126 | CRE001 | Int001 and | Int001 inserted between 27-28 bp of SEQ ID NO: 21, Int005 |
| | | Int005 | inserted between 588-589 bp of SEQ ID NO: 21 |

### Example 7

In Vivo Study of Constructs with Introns in Wild Type Mice Using AAV9 Vector

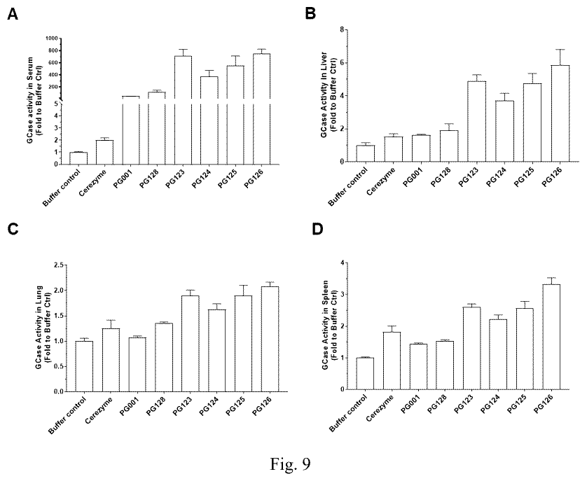

As with AAV8 products, in order to further increase the therapeutic potential of AAV9 product that express GCase, endogenous GBA1 introns of Int001, Int002, Int005 or a combination of Int001 and Int005 were inserted into the expression cassette of mGBA-C110 under the universal promoter CRE001 as mGBAi1-C110 (PG123), mGBAi2-C110 (PG124), mGBAi5-C110 (PG125) and mGBAi1i5-C110 (PG126). Constructs of PG001, PG123, PG124, PG125 and PG126 were packaged as AAV9 and proceeded for in vivo evaluation in wild type mice. These AAV9 products along with reference product of PG128, which was constructed with the sequence of SEQ ID NO:1 (149 bp-3806 bp) as described in patent U.S. Ser. No. 10/837,028B2, were injected into wild type mice at the dosage of 2E12 vg/kg by tail vein injection. Buffer control group and enzyme replacement therapy (Cerezyme) group were also included in this study. AAV9 products of PG123, PG124, PG125 and PG126 showed higher GCase activity in both serum and tissue lysates compared with buffer control, PG001 and reference product group of PG128. GCase activity in all groups from serum and different tissue lysates were highly consistent (FIG. 9). Thus, the novel designed candidates for AAV9 product could increase GCase activity in both serum and tissues, which suggest their superior therapeutic potential for Gaucher disease, and also the Parkinson's disease and Alzheimer's disease. AAV9 could cross the blood-brain-barrier and deliver GBA1 gene to the CNS efficiently to enable the expression of GCase, which have the potential to alleviate the neurological symptoms and benefit type II and III Gaucher disease, as well as Parkinson's disease and Alzheimer's disease.

1. An expression construct comprising a transcription regulatory element operably linked to a polynucleotide sequence of interest, wherein the expression construct comprises a promoter, and an enhancer, the enhancer is upstream of the promoter,
wherein the expression construct comprises the elements, in a 5′ to 3′ direction:
a) Enhance 3, the Enhance 3 is optional;
b) Enhance 2;
c) Enhance 1; and
d) promoter;
the Enhance 1 comprises all or a portion of a sequence comprising at least one selected from the sequence consisting of SEQ ID NOs: 15 and 13, and at least 80%, at least 85%, at least 90%, at least 95%, at least 99%, at least 99.5%, or at least 99.8% identical to SEQ ID NOs: 13 and 15, the all or the portion of the sequence retains the functionality of enhancer, and
the Enhance 2 comprises all or a portion of a sequence comprising at least one selected from the sequence consisting of SEQ ID NOs: 8-11 and 13, and at least 80%, at least 85%, at least 90%, at least 95%, at least 99%, at least 99.5%, or at least 99.8% identical to SEQ ID NOs: 8-11 and 13, the all or the portion of the sequence retains the functionality of enhancer.

2. The expression construct of claim 1, wherein the Enhance 3 comprises all or a portion of a sequence comprising at least one selected from the sequence consisting of SEQ ID NOs: 8-11, and at least 80%, at least 85%, at least 90%, at least 95%, at least 99%, at least 99.5%, or at least 99.8% identical to SEQ ID NOs: 8-11, the all or the portion of the sequence retains the functionality of enhancer.

3. The expression construct of claim 1, wherein the promoter comprises all or a portion of a sequence selected from the sequence consisting of SEQ ID NOs: 2-6, and at least 80%, at least 85%, at least 90%, at least 95%, at least 99%, at least 99.5%; or at least 99.8% identical to SEQ ID NOs: 2-6, the all or the portion of the sequence retains the functionality of promoter;

4. The expression construct of claim 3, wherein the promoter comprises all or a portion of a sequence selected from the sequence consisting of SEQ ID NOs: 3-6, and at least 80%, at least 85%, at least 90%, at least 95%, at least 99%, at least 99.5%, or at least 99.8% identical to SEQ ID NOs: 3-6, the all or the portion of the sequence retains the functionality of promoter;

5. The expression construct of claim 1, wherein the Enhance 1 comprises all or a portion of a sequence comprising at least one selected from the sequence consisting of SEQ ID NOs: 13 and 15, and at least 80%, at least 85%, at least 90%, at least 95%, at least 99%, at least 99.5%, or at least 99.8% identical to SEQ ID NOs: 13 and 15, the all or the portion of the sequence retains the functionality of enhancer;
the Enhance 2 comprises all or a portion of a sequence comprising at least one selected from the sequence consisting of SEQ ID NOs: 8-9, and at least 80%, at least 85%, at least 90%, at least 95%, at least 99%, at least 99.5%, or at least 99.8% identical to SEQ ID NOs: 8-9, the all or the portion of the sequence retains the functionality of enhancer.

6. The expression construct of claim 5, wherein the Enhance 1 comprises all or a portion of a sequence comprising at least one selected from the sequence consisting of SEQ ID NOs: 13 and 15, and at least 80%, at least 85%, at least 90%, at least 95%, at least 99%, at least 99.5%, or at least 99.8% identical to SEQ ID NOs: 13 and 15, the all or the portion of the sequence retains the functionality of enhancer;
the Enhance 2 comprises all or a portion of a sequence comprising SEQ ID NO: 8, and at least 80%, at least 85%, at least 90%, at least 95%, at least 99%, at least 99.5%, at least 99.8% identical to SEQ ID NOs: 8, the all or the portion of the sequence retains the functionality of enhancer.

7. The expression construct of claim 1, wherein the expression construct further comprises an untranslated intron region.

8. The expression construct of claim 7, wherein untranslated intron region comprises all or a portion of a sequence comprising at least one selected from the sequence consisting of SEQ ID NOs: 24-43, and at least 80%, at least 85%, at least 90%, at least 95%, at least 99%, at least 99.5%, at least 99.8% identical to SEQ ID NOs: 24-43.

9. The expression construct of claim 7, wherein untranslated intron region is operably linked to 5′ terminal of the polynucleotide sequence of interest.

10. The expression construct of claim 7, wherein untranslated intron region is located between 5′ and 3′ terminal of the polynucleotide sequence of interest_{∘}

11. A vector comprising the expression construct of claim 1.

12. The vector of claim 11, wherein the vector is viral vector.

13. The vector of claim 12, wherein the vector further comprises two adeno-associated virus inverted terminal repeats (ITR) sequences flanking the expression construct.

14. An adeno-associated virus (AAV) comprising the vector of claim 11 and capsid protein.

15. The AAV of claim 14, wherein
the AAV is selected from the group consisting of: serotype AAV1, AAV2, AAV3, AAV3B, AAV4, AAV5, AAV6, AAV7, AAV8, AAV9, AAV10, AAV11, AAV12, AAV13, AAVrh10, AAVhu37 or any one of the AAV serotypes isolated from human and nonhuman mammalians or variant thereof.

16. A composition comprising the
AAV of claim 14 and a pharmaceutically acceptable excipient.

17. A method for treatment of a disease or condition in a subject, comprising administering an effective amount of the AAV of claim 14 to the subject.

18. (canceled)

19. The vector of claim 12, wherein the vector is AAV vector.

20. The vector of claim 13, wherein the vector further comprises a poly A sequence.
